(19) United States
(12) Patent Application Publication
(10) Pub. No.: US 2026/0020428 A1
(43) Pub. Date: Jan. 15, 2026

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: Eun-Joung CHOI, Gyeonggi-do (KR); HaeYeon KIM, Gyeonggi-do (KR); Seung-Hyun YOON, Gyeonggi-do (KR); So-Mi PARK, Gyeonggi-do (KR)

(21) Appl. No.: 19/246,985

(22) Filed: Jun. 24, 2025

(30) Foreign Application Priority Data
Jul. 15, 2024 (KR) 10-2024-0092942
Jun. 5, 2025 (KR) 10-2025-0074033

(51) Int. Cl. H10K 50/11; C07D 471/04; C09K 11/06; H10K 50/19; H10K 85/60

(52) U.S. Cl. CPC H10K 50/11; C07D 471/04; C09K 11/06; H10K 50/19; H10K 85/615; H10K 85/633; H10K 85/636; H10K 85/654; H10K 85/6572; H10K 85/6574; H10K 85/658; C09K 2211/1044

(57) ABSTRACT

The present disclosure relates to an organic electroluminescent compound represented by Formula 1, an organic electroluminescent material, an N-type charge-generating material, and an organic electroluminescent device comprising the same. By comprising the organic electroluminescent compound, the organic electroluminescent material, or the N-type charge-generating material of the present disclosure, an organic electroluminescent device having improved luminous efficiency and/or lifetime characteristics can be provided.

### TECHNICAL FIELD

The present disclosure relates to an organic electroluminescent compound and an organic electroluminescent device comprising the same.

### BACKGROUND ART

The TPD/Alq3 bilayer small-molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. At present, OLEDs primarily use phosphorescent materials having excellent luminous efficiency in panel implementation. In many applications such as TVs and lightings, high OLED efficiency is still required. An OLED having long lifespan characteristics is required for long-term use and high display resolution.

Korean Patent Application Laid-Open No. 2027-0105040 discloses an phenanthroline derivatives, but does not specifically disclose the particular compound claimed in the present disclosure. In addition, there is a continuous need to develop light-emitting materials that exhibit improved performance compared to previously disclosed compounds, such as enhanced driving voltage, luminous efficiency, power efficiency, and/or lifetime characteristics.

### DISCLOSURE OF INVENTION

### Technical Problem

The objective of the present disclosure is to provide an organic electroluminescent compound having a novel structure that is suitable for application in organic electroluminescent devices. Another objective of the present disclosure is to provide an organic electroluminescent device exhibiting high luminous efficiency and/or long lifetime characteristics. A further objective of the present disclosure is to provide an organic electroluminescent material or an N-type charge-generating material capable of producing an organic electroluminescent device with high luminous efficiency and/or improved lifetime characteristics.

### Solution to Problem

As a result of intensive studies to solve the technical problems, the present inventors found that the above objective can be achieved by an organic electroluminescent compound represented by the following Formula 1, and an organic electroluminescent material, an N-type charge-generating material, and an organic electroluminescent device comprising the same.

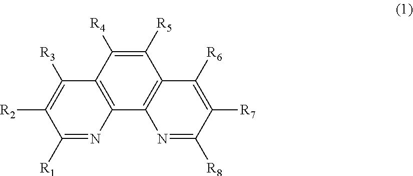

In Formula 1,
R₁ represents hydrogen, deuterium, a halogen, a cyano, a substituted or unsubstituted phenyl, a substituted or unsubstituted biphenyl, a substituted or unsubstituted terphenyl, a substituted or unsubstituted (C₁-C₃₀)alkyl, or a substituted or unsubstituted (3- to 30-membered)heteroaryl; and
R₂ to R₈ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or HAr defined by the following Formula 1-a; or may be linked to an adjacent substituent(s) to form a ring(s);
with a proviso that at least one of R₂ to R₈ is represented by HAr defined by Formula 1-a,

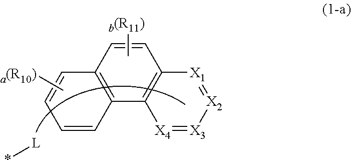

wherein X₁ to X₄ each independently represent N or CR₉;
L represents a single bond, a substituted or unsubstituted (C₆-C₃₀)arylene, or a substituted or unsubstituted (3- to 30-membered)heteroarylene;
R₉ to R₁₁ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or are bonded to L; or may be linked to an adjacent substituent(s) to form a ring(s); and
a represents an integer of 1 to 4, b represents an integer of 1 or 2, and if a and b are integers of 2 or more, each a and each b may be the same as or different from each other,
with a proviso that if R₈ is HAr in Formula 1, and at least one of X₁ to X₄ of Formula 1-a is N, R₁ is hydrogen, deuterium, a halogen, a cyano, a substituted or unsubstituted (C₁-C₃₀)alkyl, a substituted or unsubstituted phenyl, a substituted or unsubstituted biphenyl, or a substituted or unsubstituted terphenyl.

### Advantageous Effects of Invention

An organic electroluminescent compound according to the present disclosure exhibits performance suitable for use in organic electroluminescent devices. In addition, by comprising the compound according to the present disclosure as an organic electroluminescent material or an N-type charge-generating material, it is possible to provide an organic electroluminescent device exhibiting higher luminous efficiency and/or improved lifetime characteristics compared to conventional organic electroluminescent devices, and to manufacture a display device or lighting device using the same.

### MODE FOR INVENTION

Hereinafter, the present disclosure will be described in detail. However, the following description is intended to explain the present disclosure, and is not meant in any way to restrict the scope of the present disclosure.

The “organic electroluminescent compound” in the present disclosure is a compound that may be used in an organic electroluminescent device, and may be comprised in any layer constituting an organic electroluminescent device, as necessary.

The “organic electroluminescent material” in the present disclosure is a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a charge-generating material, an N-type charge-generating material, a P-type charge-generating material, a light-emitting auxiliary material, an electron-blocking material, a light-emitting material (including a host material and a dopant material), an electron buffer material, a hole-blocking material, an electron transport material, an electron injection material, etc.

Herein, the term “(C₁-C₃₀)alkyl” is meant to refer to a linear or branched alkyl having 1 to 30 carbon atoms constituting a chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C₃-C₃₀)cycloalkyl” is meant to refer to a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” in the present disclosure is meant to refer to a saturated or partially unsaturated monocyclic or polycyclic ring-shaped hydrocarbon substituent having 3 to 7, preferably 5 to 7 ring backbone atoms, and including at least one heteroatom selected from the group consisting of B, N, O, S, Si, and P, preferably at least one heteroatom selected from the group consisting of O, S, and N. The above heterocycloalkyl may include tetrahydrofuran, pyrrolidine, thiolane, tetrahydropyran, etc.

The “(C₆-C₃₀)aryl” or “(C₆-C₃₀)arylene” in the present disclosure refer to a monocyclic or fused ring radical derived from an aromatic hydrocarbon having 6 to 30 ring backbone carbon atoms, and may be partially saturated. The number of the ring backbone carbon atoms is preferably 6 to 20, more preferably 6 to 15. The above aryl may comprise a spiro structure. The above aryl may include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. Specifically, the aryl may include o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The “(3- to 30-membered)heteroaryl” or “(3- to 30-membered)heteroarylene” in the present disclosure refer to an aryl group or arylene group having 3 to 30 ring backbone atoms and including at least one heteroatom(s) selected from the group consisting of B, N, O, S, Si, and P. Herein, the number of ring backbone atoms is preferably 3 to 30, and more preferably 5 to 20. The number of heteroatoms is preferably 1 to 4. The above heteroaryl or heteroarylene may be a monocyclic ring or a fused ring condensed with at least one benzene ring, and may be partially saturated.

In addition, the above heteroaryl or heteroarylene may be one formed by linking at least one heteroaryl or aryl group to a heteroaryl group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzofuroquinolinyl, benzofuroquinazolinyl, benzofuronaphthyridinyl, benzofuropyrimidinyl, naphthofuropyrimidinyl, benzothienoquinoline, benzothienoquinazolinyl, benzothienonaphthyridinyl, benzothienopyrimidinyl, naphthienopyrimidinyl, pyrimidoindolyl, benzopyrimidoindolyl, benzofuropyrazinyl, naphthofuropyrazinyl, benzothienopyrazinyl, naphthienopyrazinyl, pyrazinoindolyl, benzopyrazinoindolyl, benzimidazolyl, benzothiazolyl, benzisothiazolyl, benzisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolyl, thiochromenoquinazolyl, dimethylbenzopyrimidinyl, indolocarbazolyl, indenocarbazolyl, etc. More specifically, the heteroaryl may include 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into a heteroaryl(ene) with electronic properties and a heteroaryl(ene) with hole properties. A heteroaryl(ene) with electronic properties is a substituent that is relatively rich in electrons in the parent nucleus, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene) with hole properties is a substituent that is relatively electron-deficient in the parent nucleus, for example, a substituted or unsubstituted carbazolyl, a substituted or unsubstituted dibenzofuranyl, a substituted or unsubstituted dibenzothiophenyl, etc.

Herein, “a fused ring group of a (C₃-C₃₀) aliphatic ring(s) and a (C₆-C₃₀) aromatic ring(s)” is meant to be a functional group of a ring in which at least one aliphatic ring having 3 to 30 ring backbone carbon atoms, preferably 3 to 25 ring backbone carbon atoms, and more preferably 3 to 18 ring backbone carbon atoms, is fused with at least one aromatic ring having 6 to 30 ring backbone carbon atoms, preferably 6 to 25 ring backbone carbon atoms, and more preferably 6 to 18 ring backbone carbon atoms. Specific examples of the fused ring group include a fused ring group of one or more benzene and one or more cyclohexane, or a fused ring group of one or more naphthalene and one or more cyclopentane, etc. Herein, the carbon atom of the fused ring group of a (C₃-C₃₀) aliphatic ring(s) and a (C₆-C₃₀) aromatic ring(s) may be replaced with one or more heteroatoms selected from B, N, O, S, Si, and P, preferably one or more heteroatoms selected from N, O, and S. Herein, “halogen” includes F, Cl, Br, and I.

In addition, “ortho-” (“o-”), “meta-” (“m-”), and “para” (“p-”) are prefixes which each represent the relative positions of substituents. The prefix “ortho-” indicates that two substituents are adjacent to each other, and for example, when two substituents in a benzene derivative occupy positions 1 and 2, this is called an “ortho” configuration. The prefix “meta” indicates that two substituents are at positions 1 and 3, and for example, when two substituents in a benzene derivative occupy positions 1 and 3, this is called a “meta-” configuration. The prefix “para-” indicates that two substituents are at positions 1 and 4, and for example, when two substituents in a benzene derivative occupy positions 1 and 4, this is called a “para-” configuration.

Herein, “a ring formed by being linked to an adjacent substituent(s)” means that at least two adjacent substituents are linked to or fused with each other to form a substituted or unsubstituted, mono- or polycyclic, (3- to 30-membered) alicyclic or aromatic ring, or a combination thereof. Preferably, the ring may be a substituted or unsubstituted, mono- or polycyclic, (5- to 25-membered) alicyclic or aromatic ring, or a combination thereof. In addition, the ring may contain at least one heteroatom selected from B, N, O, S, Si, and P, preferably at least one heteroatom selected from N, O, and S. According to one embodiment of the present disclosure, the number of ring backbone carbon atoms is 5 to 20, and according to another embodiment of the present disclosure, the number of ring backbone carbon atoms is 5 to 15. For example, the fused ring may be in the form of a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring or a substituted or unsubstituted carbazole ring, etc.

Herein, “substituted” in the expression “substituted or unsubstituted” means that a hydrogen atom in a certain functional group is replaced with another atom or another functional group, i.e., a substituent, and this also includes substitution by a group in which two or more of the substituents are connected. Unless otherwise specified, the substituent may replace hydrogen at a position where the substituent can be substituted without limitation, and when two or more hydrogen atoms in a certain functional group are each replaced with a substituent, each substituent may be the same as or different from each other. The maximum number of substituents that can be substituted for a certain functional group may be the total number of valences that can be substituted for each atom forming the functional group. Herein, the substituted phenyl, the substituted biphenyl, the substituted terphenyl, the substituted alkyl, the substituted alkenyl, the substituted aryl, the substituted arylene, the substituted heteroaryl, the substituted heteroarylene, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, and the substituted fused ring group of an aliphatic ring(s) and an aromatic ring(s), each independently may be substituted with at least one selected from the group consisting of deuterium, a halogen, a cyano, a carboxyl, a nitro, a hydroxyl, a (C₁-C₃₀)alkyl, a halo(C₁-C₃₀)alkyl, a (C₂-C₃₀)alkenyl, a (C₂-C₃₀)alkynyl, a (C₁-C₃₀)alkoxy, a (C₁-C₃₀)alkylthio, a (C₃-C₃₀)cycloalkyl, a (C₃-C₃₀)cycloalkenyl, a (3-to 7-membered)heterocycloalkyl, a (C₆-C₃₀)aryloxy, a (C₆-C₃₀)arylthio, a (5- to 30-membered)heteroaryl unsubstituted or substituted with a (C₆-C₃₀)aryl, a (C₆-C₃₀)aryl unsubstituted or substituted with a (5- to 30-membered)heteroaryl, a tri(C₁-C₃₀)alkylsilyl, a tri(C₆-C₃₀)arylsilyl, a di(C₁-C₃₀)alkyl(C₆-C₃₀)arylsilyl, a (C₁-C₃₀)alkyldi(C₆-C₃₀)arylsilyl, a fused ring group of a (C₃-C₃₀) aliphatic ring(s) and a (C₆-C₃₀) aromatic ring(s), an amino, a mono- or di(C₁-C₃₀)alkylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3- to 30-membered)heteroarylamino, a substituted or unsubstituted (C₁-C₃₀)alkyl(3- to 30-membered)heteroarylamino, a substituted or unsubstituted (C₆-C₃₀)aryl(3- to 30-membered)heteroarylamino, a (C₁-C₃₀)alkylcarbonyl, a (C₁-C₃₀)alkoxycarbonyl, a (C₆-C₃₀)arylcarbonyl, a (C₆-C₃₀)arylphosphinyl, a di(C₆-C₃₀)arylboronyl, a di(C₁-C₃₀)alkylboronyl, a (C₁-C₃₀)alkyl(C₆-C₃₀)arylboronyl, a (C₆-C₃₀)aryl(C₁-C₃₀)alkyl, a (C₁-C₃₀)alkyl(C₆-C₃₀)aryl, and a combination thereof. According to one embodiment of the present disclosure, the substituent may be deuterium.

In the present disclosure, if a substituent is not indicated in the chemical formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In the present disclosure, in cases where a substituent is not indicated in the chemical formula or compound structure, if the substituent is not explicitly excluded, such as 0% deuterium, 100% hydrogen, and all substituents are hydrogen, hydrogen and deuterium may be used intermixed in a compound. Deuterium is one of the isotopes of hydrogen and an element with a deuteron consisting of one proton and one neutron as its nucleus. It can be represented as hydrogen-2, whose element symbol can also be written as D or ²H. Isotopes are atoms with the same atomic number (Z) but different mass numbers (A), and can also be interpreted as elements with the same number of protons but different numbers of neutrons.

In the present disclosure, “a combination thereof” refers to a combination of one or more elements from the corresponding list to form a known or chemically stable arrangement that can be envisioned by a person skilled in the art from the corresponding list. For example, alkyl and deuterium can be combined to form a partially or fully deuterated alkyl group, a halogen and alkyl can be combined to form a halogenated alkyl substituent, and a halogen, alkyl, and aryl can be combined to form a halogenated arylalkyl. For example, a preferred combination of substituents includes up to 50 atoms that are not hydrogen or deuterium, or up to 40 atoms that are not hydrogen or deuterium, or up to 30 atoms that are not hydrogen or deuterium, or in many cases, a preferred combination of substituents may comprise up to 20 atoms that are not hydrogen or deuterium.

In the formulas of the present disclosure, when there are multiple substituents represented by the same symbol, each substituent represented by the same symbol may be the same as or different from each other.

The compound represented by Formula 1 is described in more detail as follows.

In Formula 1, R₁ represents hydrogen, deuterium, a halogen, a cyano, a substituted or unsubstituted phenyl, a substituted or unsubstituted biphenyl, a substituted or unsubstituted terphenyl, a substituted or unsubstituted (C₁-C₃₀)alkyl, or a substituted or unsubstituted (3- to 30-membered)heteroaryl. According to one embodiment of the present disclosure, R₁ represents hydrogen, deuterium, a halogen, a substituted or unsubstituted phenyl, a substituted or unsubstituted biphenyl, a substituted or unsubstituted terphenyl, a substituted or unsubstituted (C₁-C₂₀)alkyl, or a substituted or unsubstituted (5- to 25-membered)heteroaryl. According to another embodiment of the present disclosure, R₁ represents hydrogen, deuterium, a halogen, a phenyl unsubstituted or substituted with deuterium, a biphenyl unsubstituted or substituted with deuterium, a terphenyl unsubstituted or substituted with deuterium, a (C₁-C₆)alkyl unsubstituted or substituted with deuterium, or (5- to 25-membered)heteroaryl unsubstituted or substituted with deuterium. For example, R₁ may be hydrogen, deuterium, a fluoro, a methyl, a phenyl, an o-biphenyl, a m-biphenyl, a p-biphenyl, a pyridyl, etc., which can be substituted with one or more deuterium.

In Formula 1, R₂ to R₈ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or HAr defined by the following Formula 1-a; or may be linked to an adjacent substituent(s) to form a ring(s); with a proviso that at least one of R₂ to R₈ is represented by HAr defined by Formula 1-a. According to one embodiment of the present disclosure, R₂ to R₈ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, or HAr defined by the following Formula 1-a. According to another embodiment of the present disclosure, R₂ to R₈ each independently represent hydrogen, deuterium, a phenyl, a pyridyl, etc., or HAr defined by the following Formula 1-a, which can be substituted with one or more deuterium.

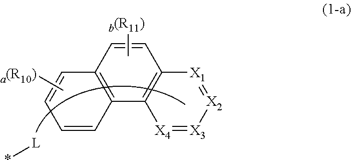

In Formula 1-a, X₁ to X₄ each independently represent N or CR₉. According to one embodiment of the present disclosure, at least one of X₁ to X₄ is N. According to another embodiment of the present disclosure, at least one or two of X₁ to X₄ are N.

In Formula 1-a, L represents a single bond, a substituted or unsubstituted (C₆-C₃₀)arylene, or a substituted or unsubstituted (3- to 30-membered)heteroarylene. According to one embodiment of the present disclosure, L represents a single bond, a substituted or unsubstituted (C₆-C₂₅)arylene, or a substituted or unsubstituted (5- to 20-membered)heteroarylene. According to another embodiment of the present disclosure, L represents a single bond, or a (C₆-C₁₈)arylene unsubstituted or substituted with deuterium. For example, L may be a single bond, a phenylene, a naphthylene, etc., which can be substituted with one or more deuterium.

In Formula 1-a, R₉ to R₁₁ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or are bonded to L; or may be linked to an adjacent substituent(s) to form a ring(s). According to one embodiment of the present disclosure, one of R₉ to R₁₁ is bonded to L, and the remaining R₉ to R₁₁ are each independently hydrogen, deuterium, a substituted or unsubstituted (C₁-C₂₀)alkyl, a substituted or unsubstituted (C₆-C₂₅)aryl, or a substituted or unsubstituted (5- to 25-membered)heteroaryl. According to another embodiment of the present disclosure, one of R₉ to R₁₁ is bonded to L, and the remaining R₉ to R₁₁ are each independently hydrogen, deuterium, a (C₁-C₁₀)alkyl unsubstituted or substituted with deuterium, or (C₆-C₁₈)aryl unsubstituted or substituted with deuterium. For example, R₉ may be hydrogen, deuterium, a methyl, a phenyl, etc., which can be substituted with one or more deuterium; R₁₀ and R₁₁ may be each independently hydrogen or deuterium; and one of R₉ to R₁₁ may be bonded to L.

In Formula 1-a, a represents an integer of 1 to 4, b represents an integer of 1 or 2, and if a and b are integers of 2 or more, each a and each b may be the same as or different from each other.

With a proviso that if R₈ is HAr in Formula 1, and at least one of X₁ to X₄ of Formula 1-a is N, R₁ is hydrogen, deuterium, a halogen, a cyano, a substituted or unsubstituted (C₁-C₃₀)alkyl, a substituted or unsubstituted phenyl, a substituted or unsubstituted biphenyl, or a substituted or unsubstituted terphenyl.

Formula 1 may be represented by at least one of the following Formulas 1-1 to 1-7.

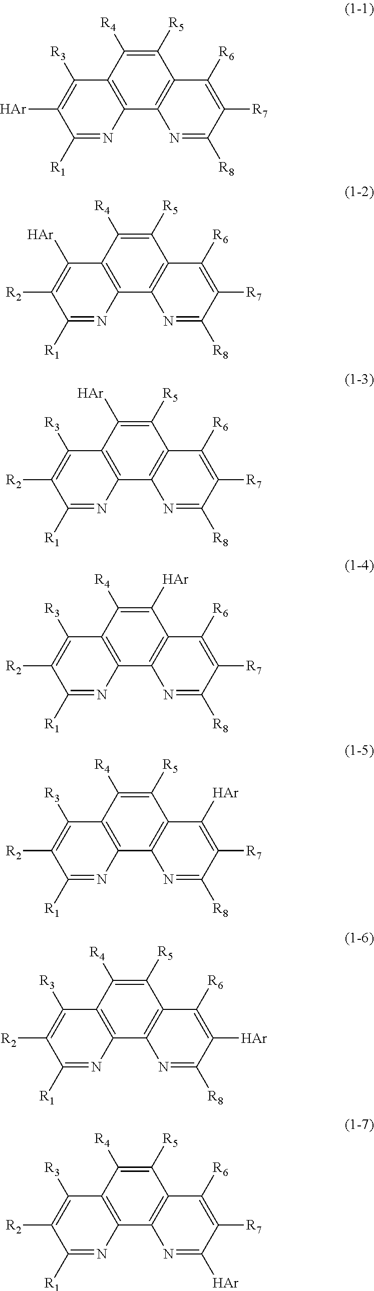

In Formulas 1-1 to 1-7, R₁ and HAr are as defined in Formula 1.

In Formulas 1-1 to 1-7, R₂ to R₈ each independently represent hydrogen, deuterium, a halogen, a cyano, a substituted or unsubstituted (C₁-C₃₀)alkyl, a substituted or unsubstituted (C₆-C₃₀)aryl, or a substituted or unsubstituted (3- to 30-membered)heteroaryl. For example, R₂ to R₈ each independently represent hydrogen, deuterium, a phenyl, a pyridyl, etc., which can be substituted with one or more deuterium.

Formula 1-a may be represented by at least one of the following Formulas 1-a-1 to 1-a-10.

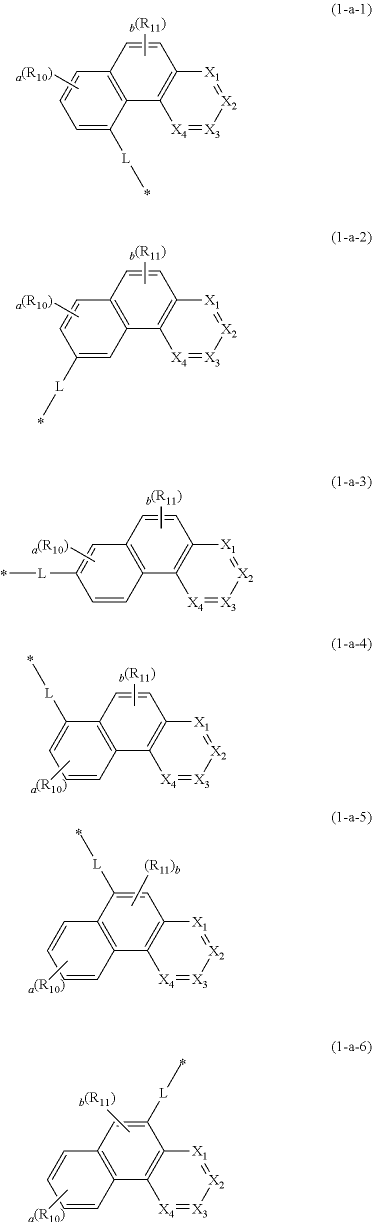

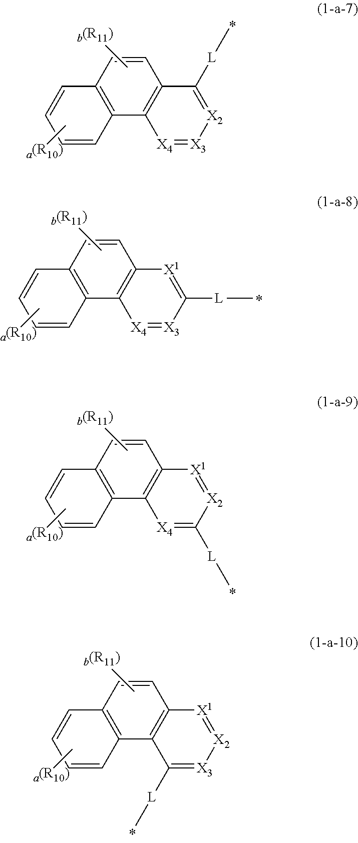

In Formulas 1-a-1 to 1-a-10, R₁₀, R₁₁, L, X₁ to X₄, a, and b are as defined in Formula 1-a.

The organic electroluminescent compound represented by Formula 1 may be at least one selected from the group consisting of the following compounds, but is not limited thereto.

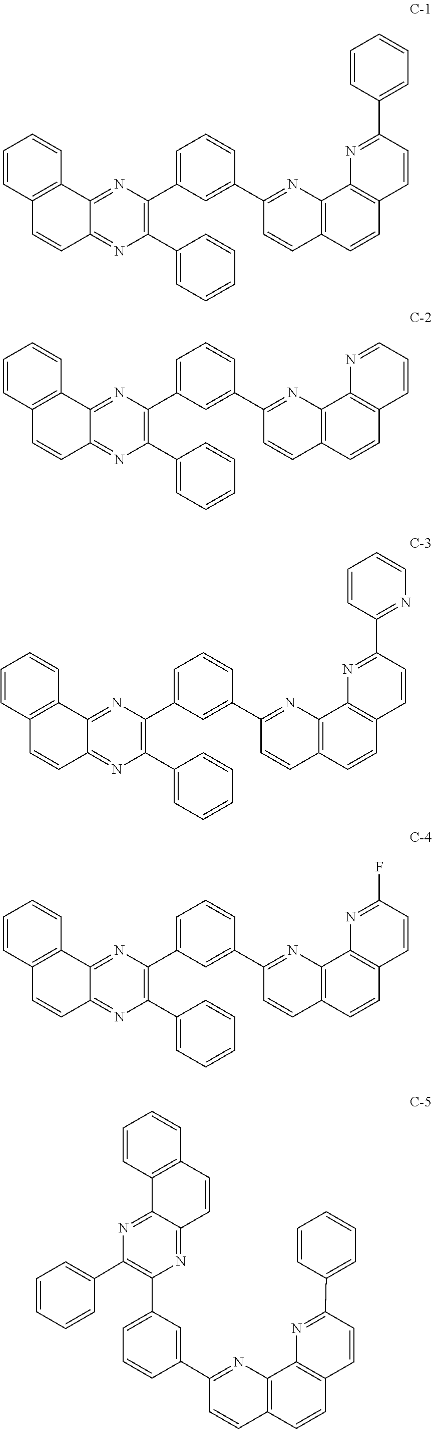

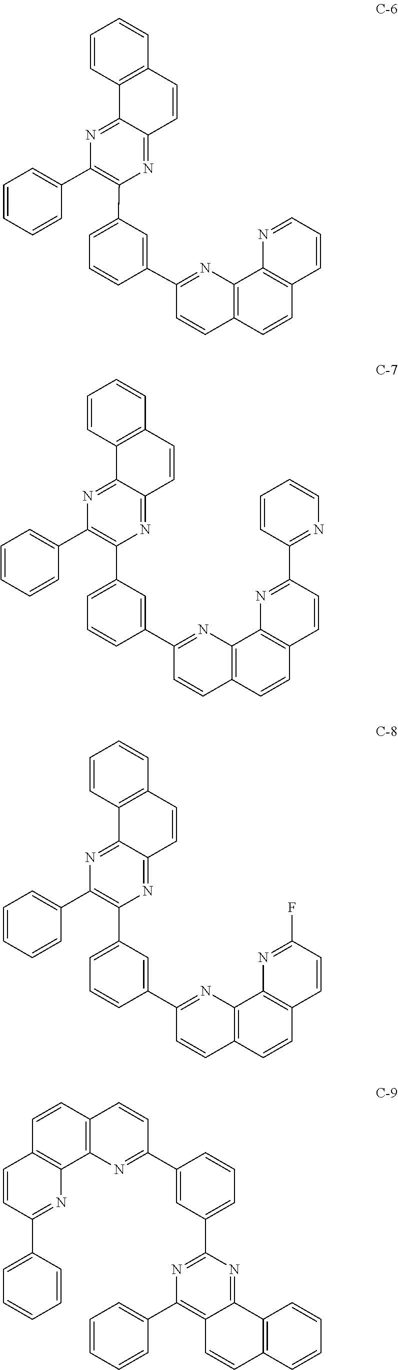 [SMILES: c1ccc(-c2nc3c(ccc4ccccc43)nc2-c2cccc(-c3ccc4ccc5cccnc5c4n3)c2)cc1 ; c1ccc(-c2nc3c(ccc4ccccc43)nc2-c2cccc(-c3ccc4ccc5ccc(-c6ccccn6)nc5c4n3)c2)cc1 ; Fc1ccc2ccc3ccc(-c4cccc(-c5nc6ccc7ccccc7c6nc5-c5ccccc5)c4)nc3c2n1 ; c1ccc(-c2ccc3ccc4ccc(-c5cccc(-c6nc(-c7ccccc7)c7ccc8ccccc8c7n6)c5)nc4c3n2)cc1]

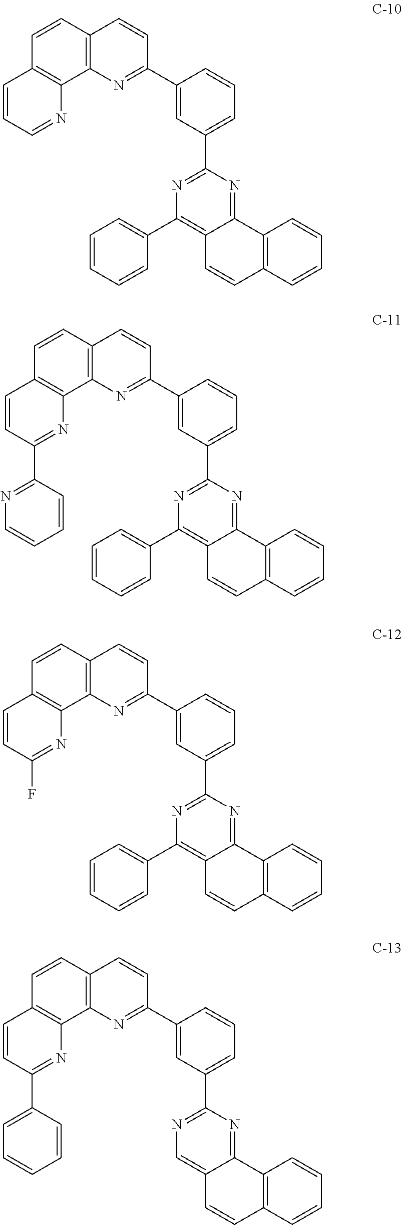

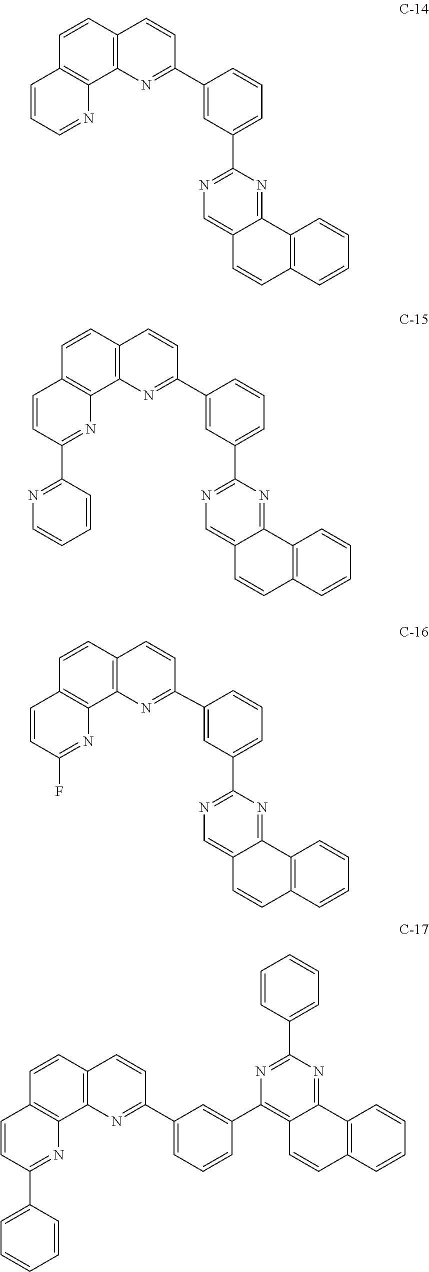 [SMILES: c1cc(-c2ccc3ccc4cccnc4c3n2)cc(-c2ncc3ccc4ccccc4c3n2)c1 ; c1ccc(-c2ccc3ccc4ccc(-c5cccc(-c6ncc7ccc8ccccc8c7n6)c5)nc4c3n2)nc1 ; Fc1ccc2ccc3ccc(-c4cccc(-c5ncc6ccc7ccccc7c6n5)c4)nc3c2n1 ; c1ccc(-c2ccc3ccc4ccc(-c5cccc(-c6nc(-c7ccccc7)nc7c6ccc6ccccc67)c5)nc4c3n2)cc1]

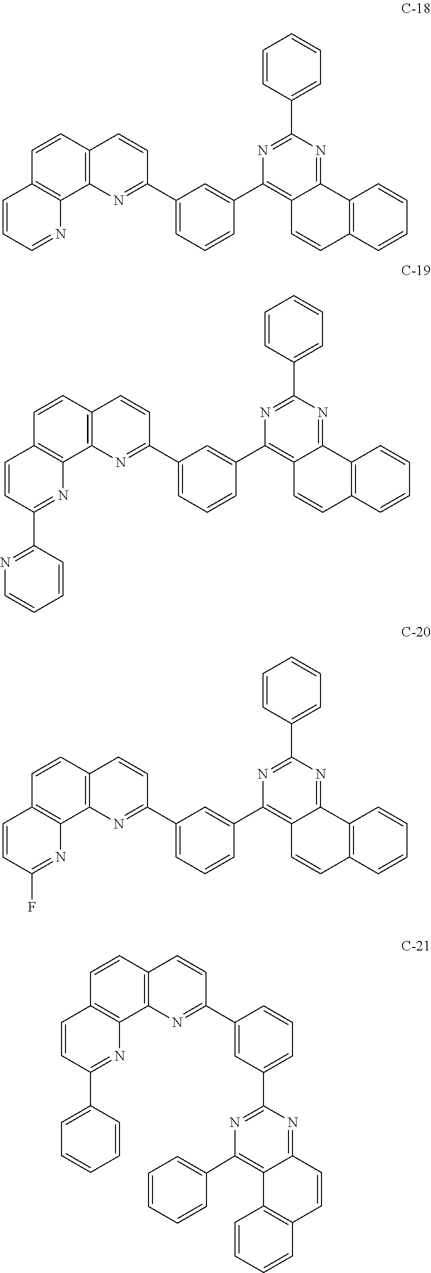 [SMILES: c1ccc(-c2nc(-c3cccc(-c4ccc5ccc6cccnc6c5n4)c3)c3ccc4ccccc4c3n2)cc1 ; c1ccc(-c2nc(-c3cccc(-c4ccc5ccc6ccc(-c7ccccn7)nc6c5n4)c3)c3ccc4ccccc4c3n2)cc1 ; Fc1ccc2ccc3ccc(-c4cccc(-c5nc(-c6ccccc6)nc6c5ccc5ccccc56)c4)nc3c2n1 ; c1ccc(-c2ccc3ccc4ccc(-c5cccc(-c6nc(-c7ccccc7)c7c(ccc8ccccc87)n6)c5)nc4c3n2)cc1]

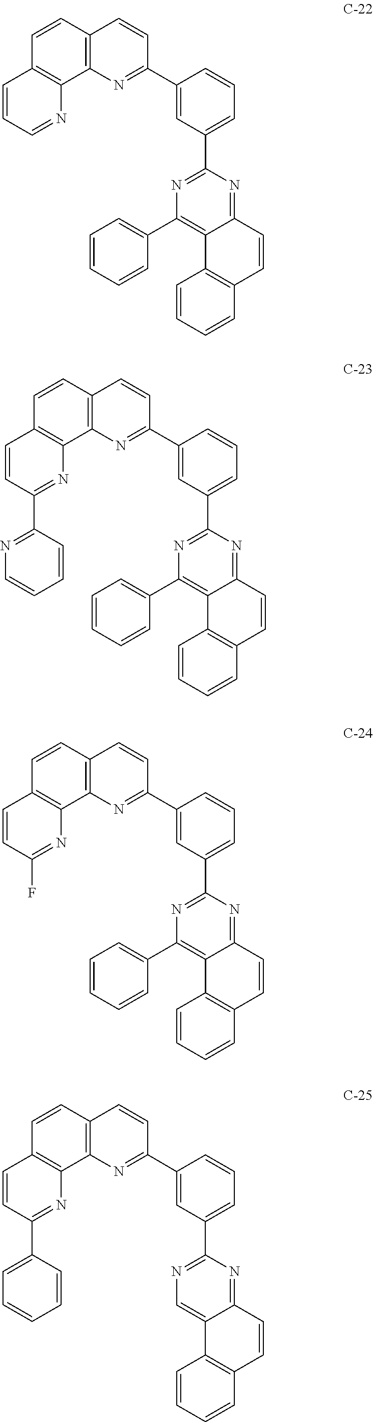 [SMILES: c1ccc(-c2nc(-c3cccc(-c4ccc5ccc6cccnc6c5n4)c3)nc3ccc4ccccc4c23)cc1 ; c1ccc(-c2nc(-c3cccc(-c4ccc5ccc6ccc(-c7ccccn7)nc6c5n4)c3)nc3ccc4ccccc4c23)cc1 ; Fc1ccc2ccc3ccc(-c4cccc(-c5nc(-c6ccccc6)c6c(ccc7ccccc76)n5)c4)nc3c2n1 ; c1ccc(-c2ccc3ccc4ccc(-c5cccc(-c6ncc7c(ccc8ccccc87)n6)c5)nc4c3n2)cc1]

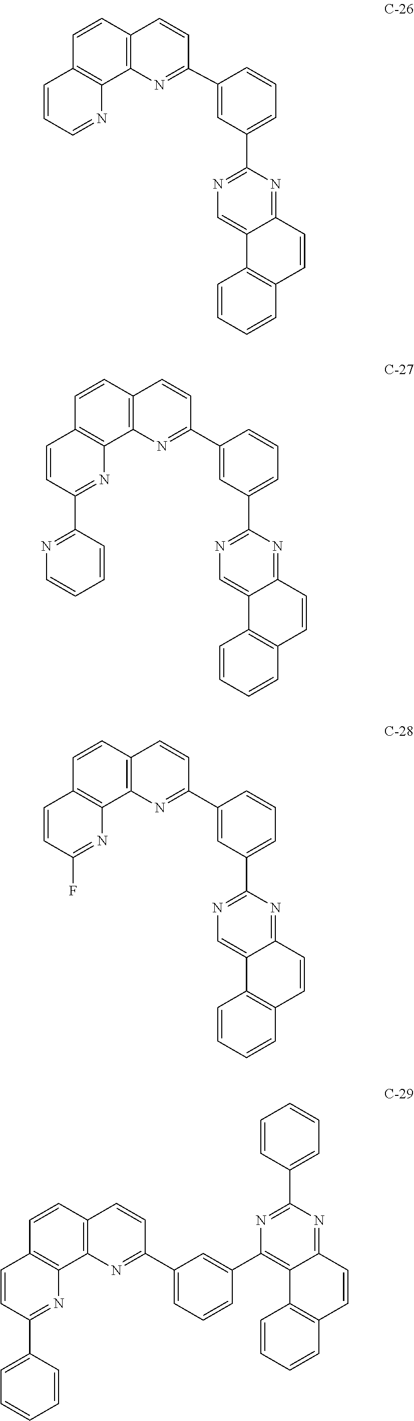 [SMILES: c1cc(-c2ccc3ccc4cccnc4c3n2)cc(-c2ncc3c(ccc4ccccc43)n2)c1 ; c1ccc(-c2ccc3ccc4ccc(-c5cccc(-c6ncc7c(ccc8ccccc87)n6)c5)nc4c3n2)nc1 ; Fc1ccc2ccc3ccc(-c4cccc(-c5ncc6c(ccc7ccccc76)n5)c4)nc3c2n1 ; c1ccc(-c2ccc3ccc4ccc(-c5cccc(-c6nc(-c7ccccc7)nc7ccc8ccccc8c67)c5)nc4c3n2)cc1]

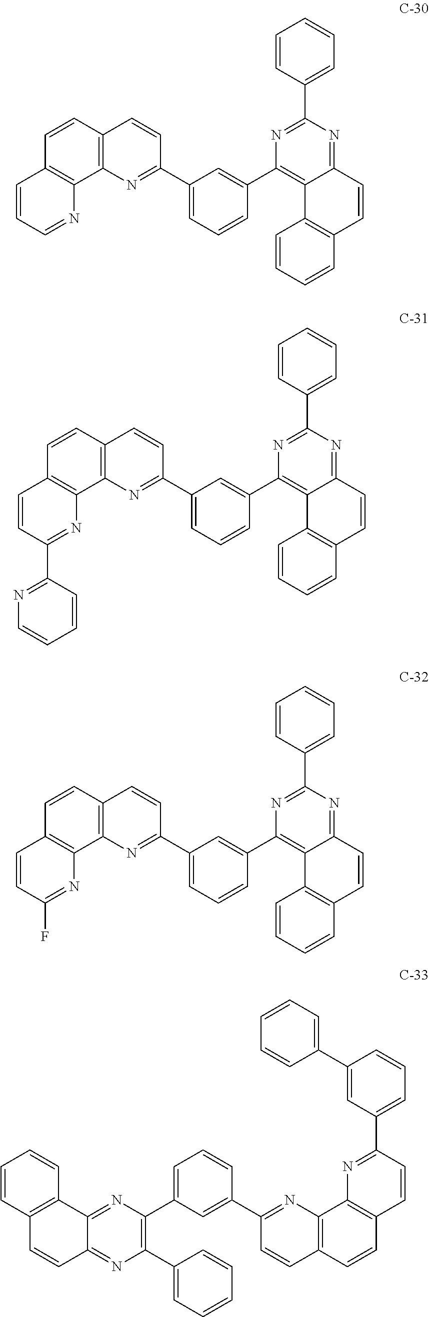

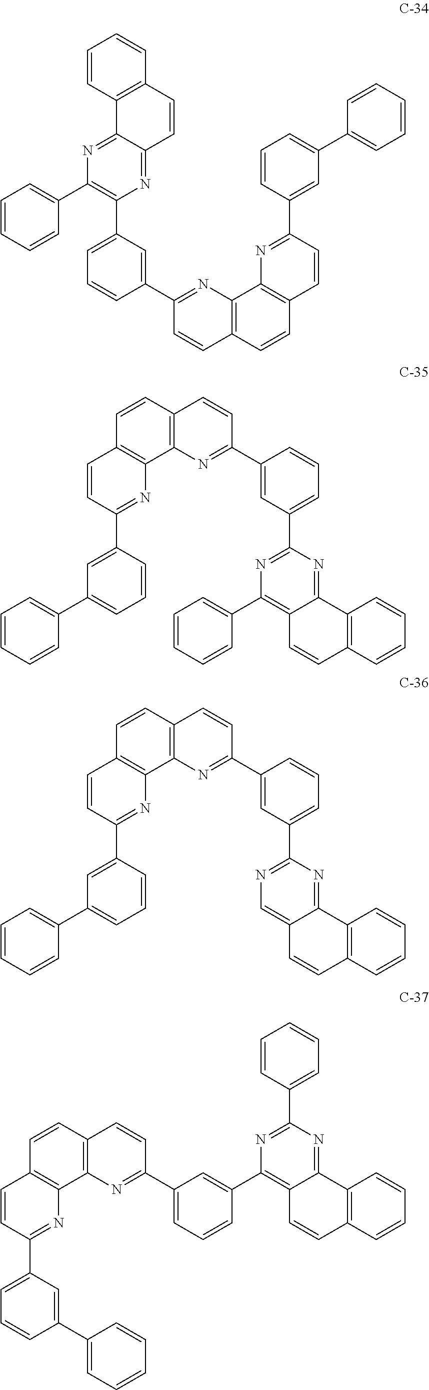

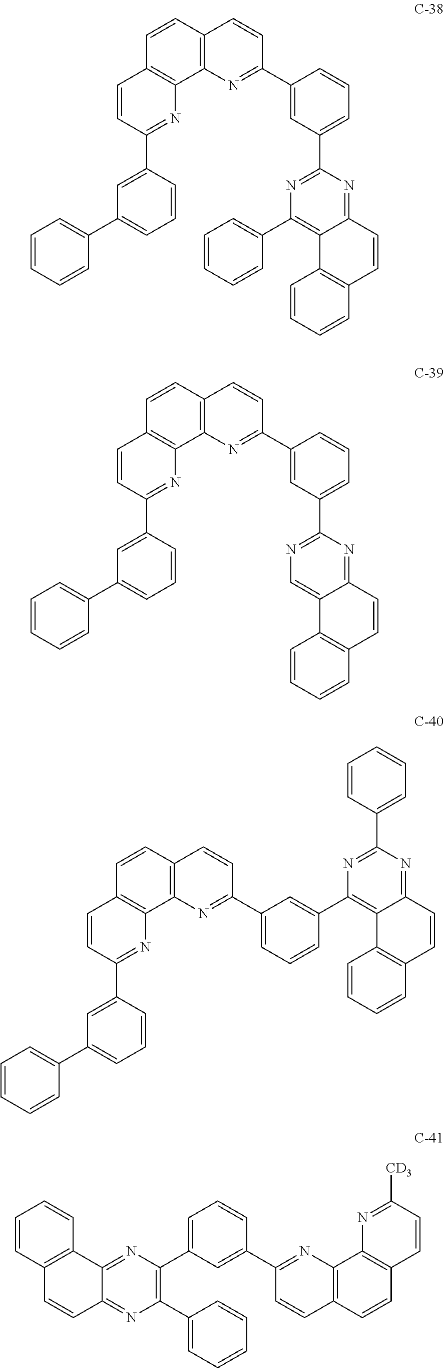 [SMILES: c1ccc(-c2cccc(-c3ccc4ccc5ccc(-c6cccc(-c7nc(-c8ccccc8)c8c(ccc9ccccc98)n7)c6)nc5c4n3)c2)cc1 ; c1ccc(-c2cccc(-c3ccc4ccc5ccc(-c6cccc(-c7ncc8c(ccc9ccccc98)n7)c6)nc5c4n3)c2)cc1 ; c1ccc(-c2cccc(-c3ccc4ccc5ccc(-c6cccc(-c7nc(-c8ccccc8)nc8ccc9ccccc9c78)c6)nc5c4n3)c2)cc1 ; [2H]C([2H])([2H])c1ccc2ccc3ccc(-c4cccc(-c5nc6c(ccc7ccccc76)nc5-c5ccccc5)c4)nc3c2n1]

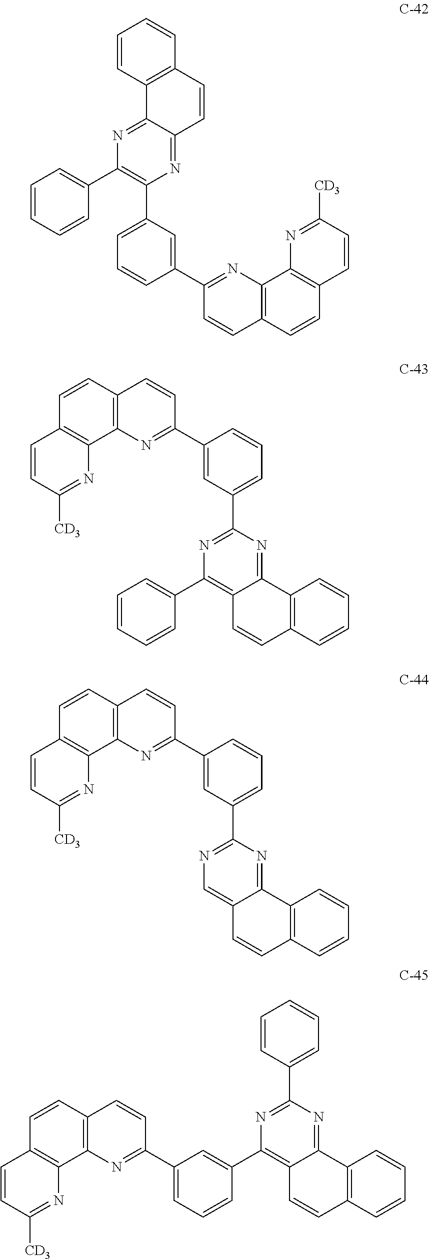

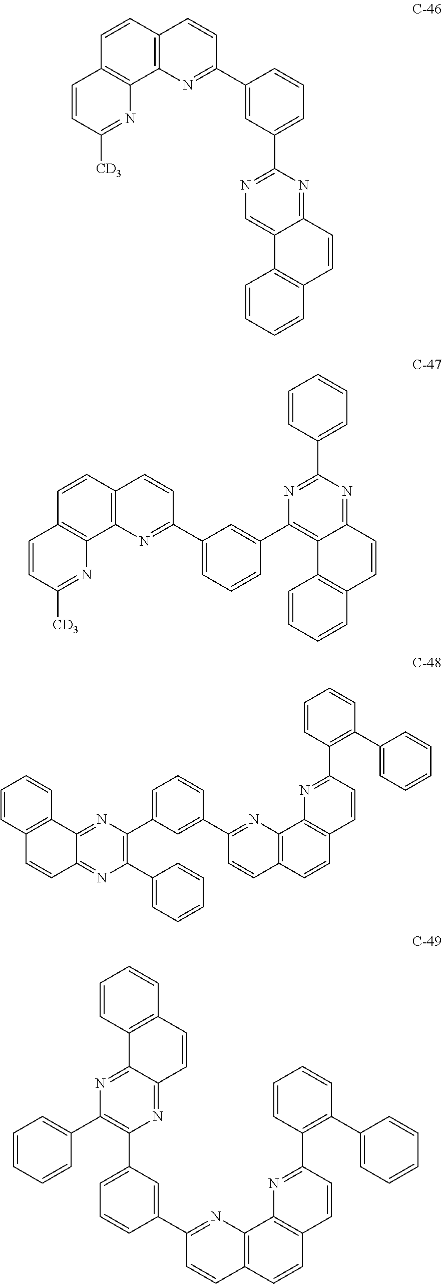 [SMILES: [2H]C([2H])([2H])c1ccc2ccc3ccc(-c4cccc(-c5ncc6c(ccc7ccccc76)n5)c4)nc3c2n1 ; [2H]C([2H])([2H])c1ccc2ccc3ccc(-c4cccc(-c5nc(-c6ccccc6)nc6ccc7ccccc7c56)c4)nc3c2n1 ; c1ccc(-c2ccccc2-c2ccc3ccc4ccc(-c5cccc(-c6nc7c(ccc8ccccc87)nc6-c6ccccc6)c5)nc4c3n2)cc1 ; c1ccc(-c2ccccc2-c2ccc3ccc4ccc(-c5cccc(-c6nc7ccc8ccccc8c7nc6-c6ccccc6)c5)nc4c3n2)cc1]

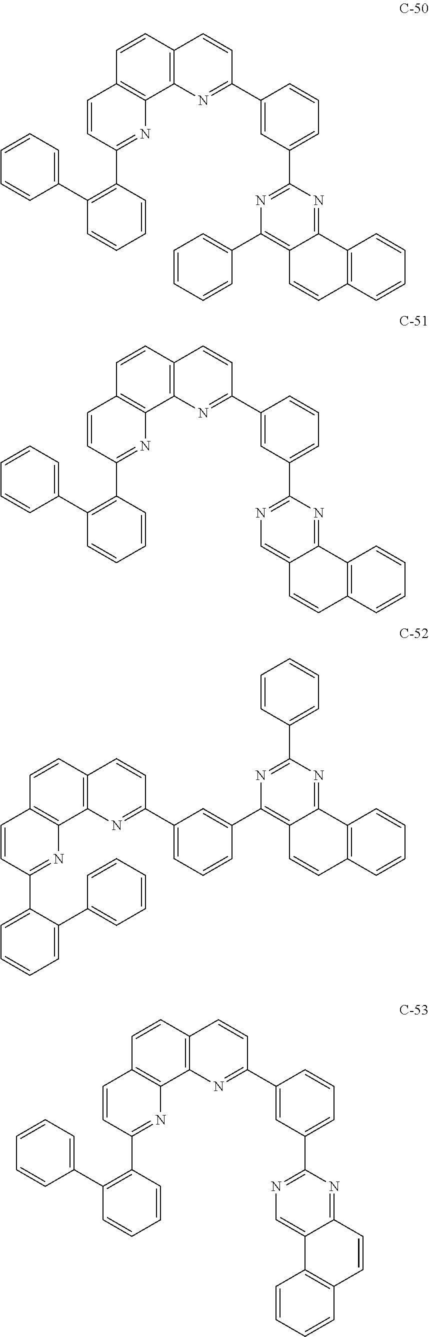

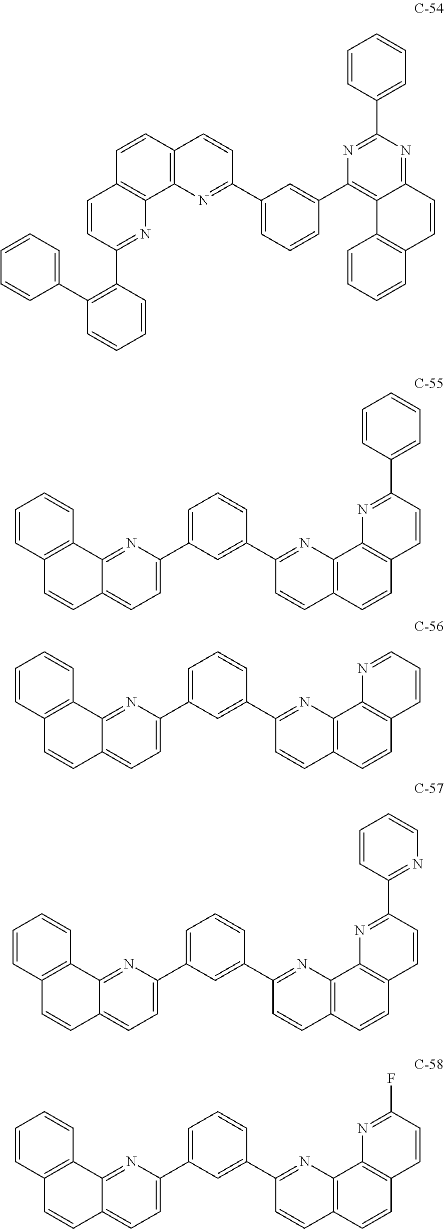 [SMILES: c1ccc(-c2nc(-c3cccc(-c4ccc5ccc6ccc(-c7ccccc7-c7ccccc7)nc6c5n4)c3)c3c(ccc4ccccc43)n2)cc1 ; c1ccc(-c2ccc3ccc4ccc(-c5cccc(-c6ccc7ccc8ccccc8c7n6)c5)nc4c3n2)cc1 ; c1cc(-c2ccc3ccc4ccccc4c3n2)cc(-c2ccc3ccc4cccnc4c3n2)c1 ; c1ccc(-c2ccc3ccc4ccc(-c5cccc(-c6ccc7ccc8ccccc8c7n6)c5)nc4c3n2)nc1 ; Fc1ccc2ccc3ccc(-c4cccc(-c5ccc6ccc7ccccc7c6n5)c4)nc3c2n1]

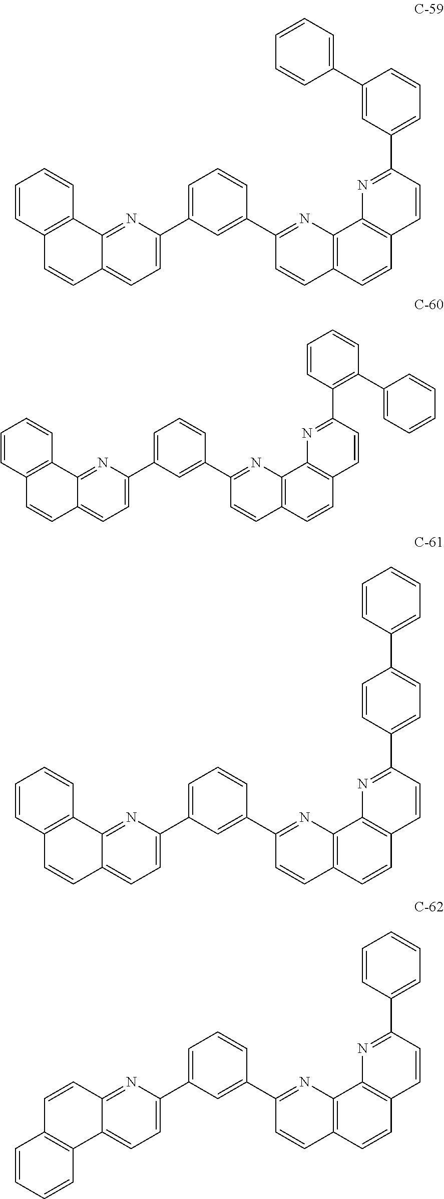 [SMILES: c1ccc(-c2cccc(-c3ccc4ccc5ccc(-c6cccc(-c7ccc8ccc9ccccc9c8n7)c6)nc5c4n3)c2)cc1 ; c1ccc(-c2ccccc2-c2ccc3ccc4ccc(-c5cccc(-c6ccc7ccc8ccccc8c7n6)c5)nc4c3n2)cc1 ; c1ccc(-c2ccc(-c3ccc4ccc5ccc(-c6cccc(-c7ccc8ccc9ccccc9c8n7)c6)nc5c4n3)cc2)cc1 ; c1ccc(-c2ccc3ccc4ccc(-c5cccc(-c6ccc7c(ccc8ccccc87)n6)c5)nc4c3n2)cc1]

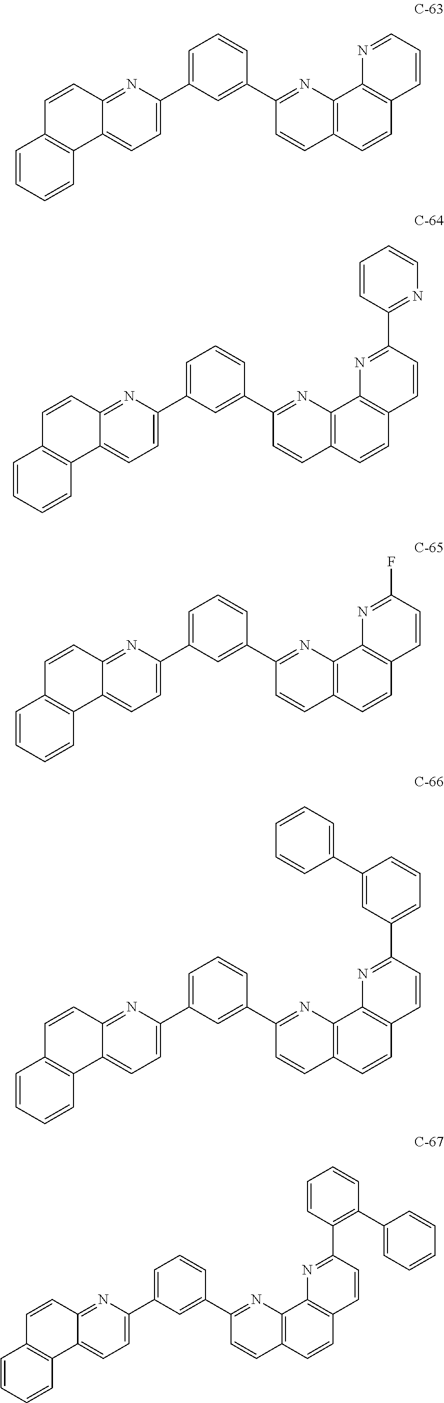 [SMILES: c1cc(-c2ccc3c(ccc4ccccc43)n2)cc(-c2ccc3ccc4cccnc4c3n2)c1 ; c1ccc(-c2ccc3ccc4ccc(-c5cccc(-c6ccc7c(ccc8ccccc87)n6)c5)nc4c3n2)nc1 ; Fc1ccc2ccc3ccc(-c4cccc(-c5ccc6c(ccc7ccccc76)n5)c4)nc3c2n1 ; c1ccc(-c2cccc(-c3ccc4ccc5ccc(-c6cccc(-c7ccc8c(ccc9ccccc98)n7)c6)nc5c4n3)c2)cc1 ; c1ccc(-c2ccccc2-c2ccc3ccc4ccc(-c5cccc(-c6ccc7c(ccc8ccccc87)n6)c5)nc4c3n2)cc1]

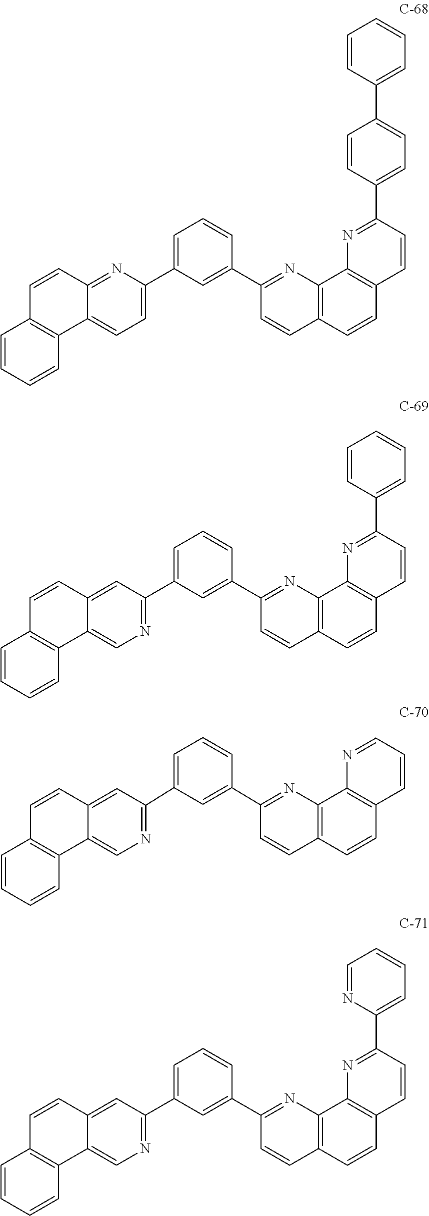 [SMILES: c1ccc(-c2ccc(-c3ccc4ccc5ccc(-c6cccc(-c7ccc8c(ccc9ccccc98)n7)c6)nc5c4n3)cc2)cc1 ; c1ccc(-c2ccc3ccc4ccc(-c5cccc(-c6cc7ccc8ccccc8c7cn6)c5)nc4c3n2)cc1 ; c1cc(-c2cc3ccc4ccccc4c3cn2)cc(-c2ccc3ccc4cccnc4c3n2)c1 ; c1ccc(-c2ccc3ccc4ccc(-c5cccc(-c6cc7ccc8ccccc8c7cn6)c5)nc4c3n2)nc1]

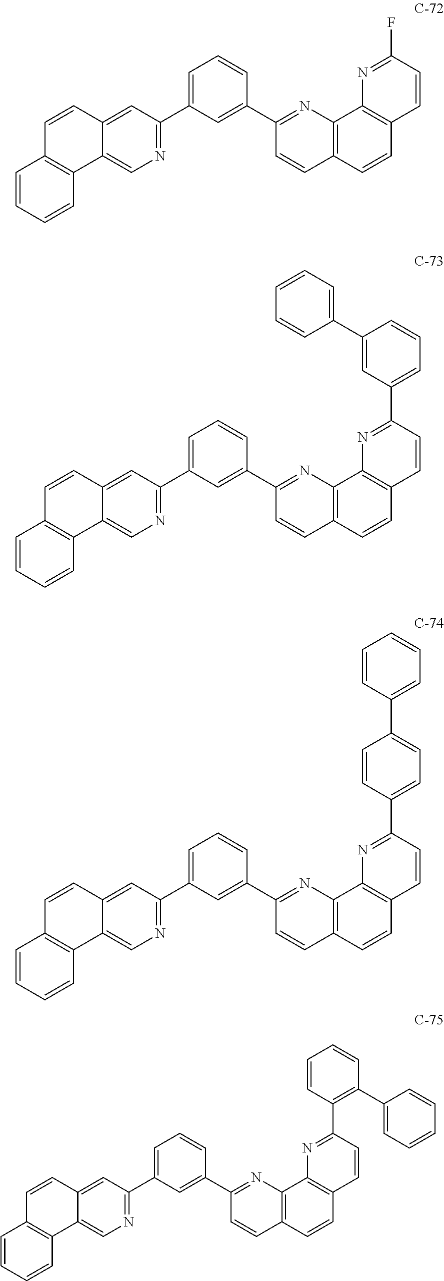 [SMILES: Fc1ccc2ccc3ccc(-c4cccc(-c5cc6ccc7ccccc7c6cn5)c4)nc3c2n1 ; c1ccc(-c2cccc(-c3ccc4ccc5ccc(-c6cccc(-c7cc8ccc9ccccc9c8cn7)c6)nc5c4n3)c2)cc1 ; c1ccc(-c2ccc(-c3ccc4ccc5ccc(-c6cccc(-c7cc8ccc9ccccc9c8cn7)c6)nc5c4n3)cc2)cc1 ; c1ccc(-c2ccccc2-c2ccc3ccc4ccc(-c5cccc(-c6cc7ccc8ccccc8c7cn6)c5)nc4c3n2)cc1]

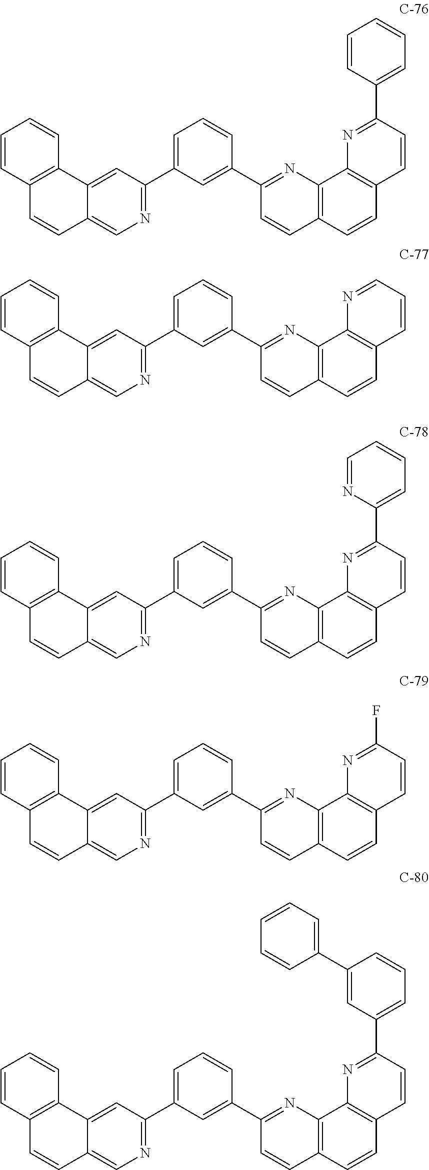

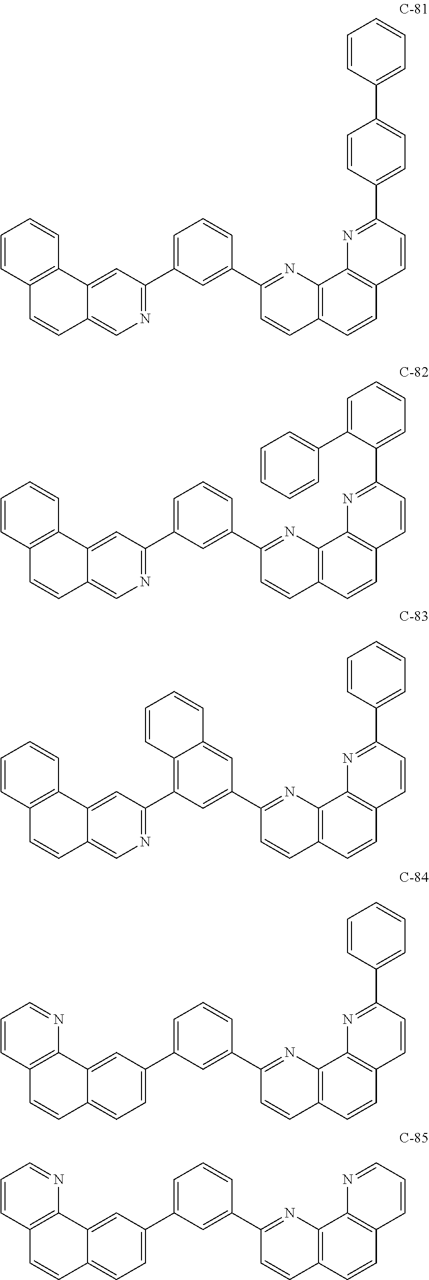 [SMILES: c1ccc(-c2ccc(-c3ccc4ccc5ccc(-c6cccc(-c7cc8c(ccc9ccccc98)cn7)c6)nc5c4n3)cc2)cc1 ; c1ccc(-c2ccccc2-c2ccc3ccc4ccc(-c5cccc(-c6cc7c(ccc8ccccc87)cn6)c5)nc4c3n2)cc1 ; c1ccc(-c2ccc3ccc4ccc(-c5cc(-c6cc7c(ccc8ccccc87)cn6)c6ccccc6c5)nc4c3n2)cc1 ; c1ccc(-c2ccc3ccc4ccc(-c5cccc(-c6ccc7ccc8cccnc8c7c6)c5)nc4c3n2)cc1 ; c1cc(-c2ccc3ccc4cccnc4c3c2)cc(-c2ccc3ccc4cccnc4c3n2)c1]

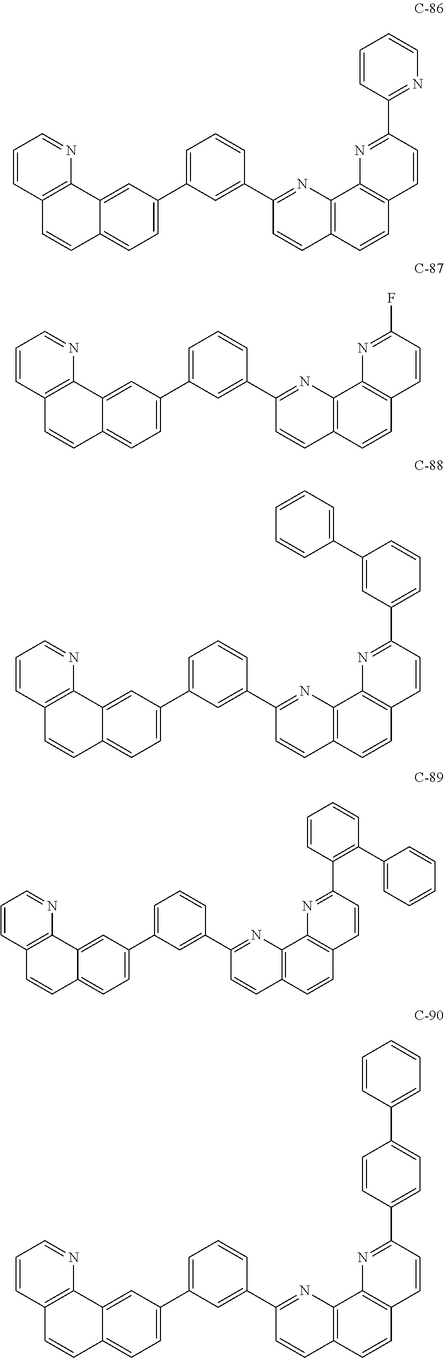

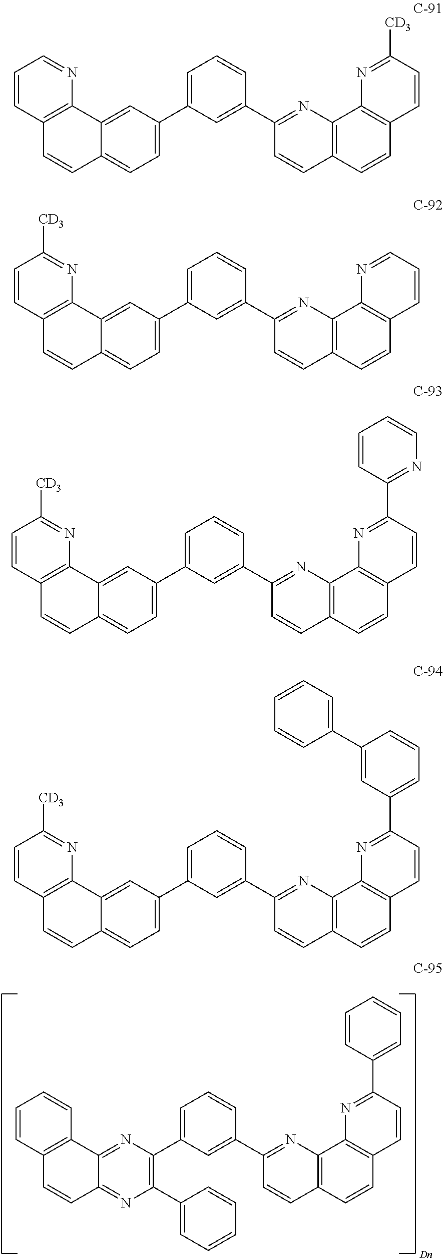

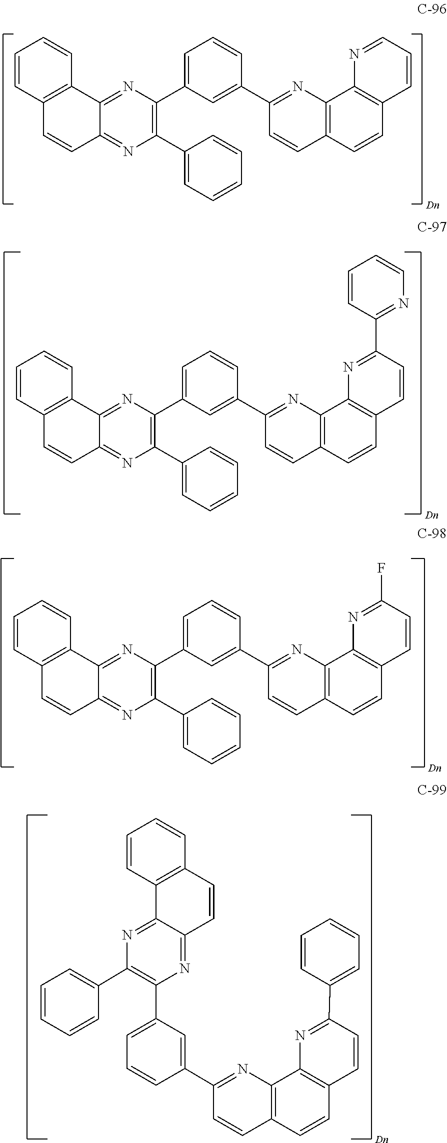

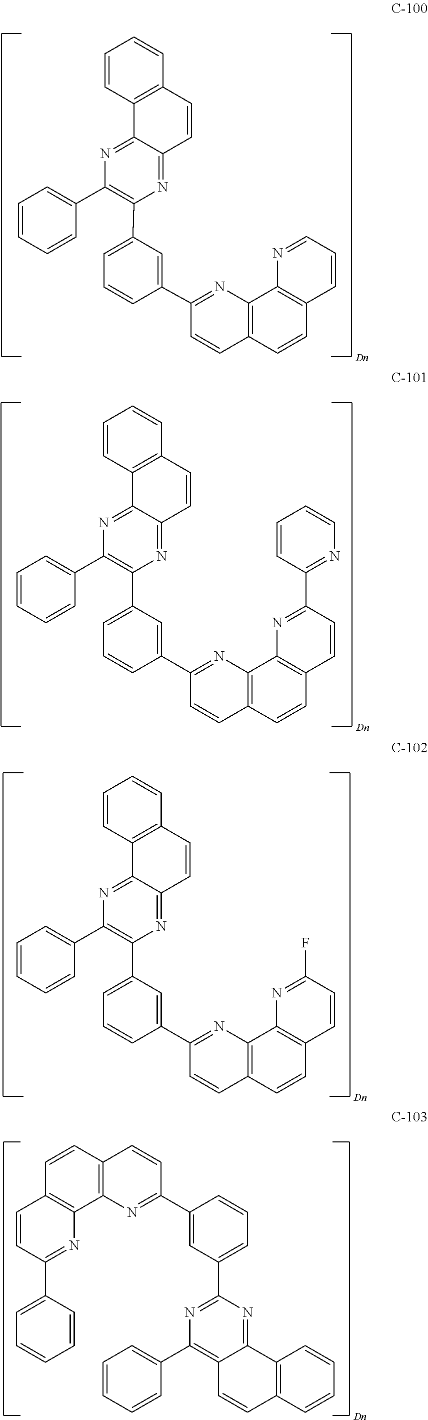

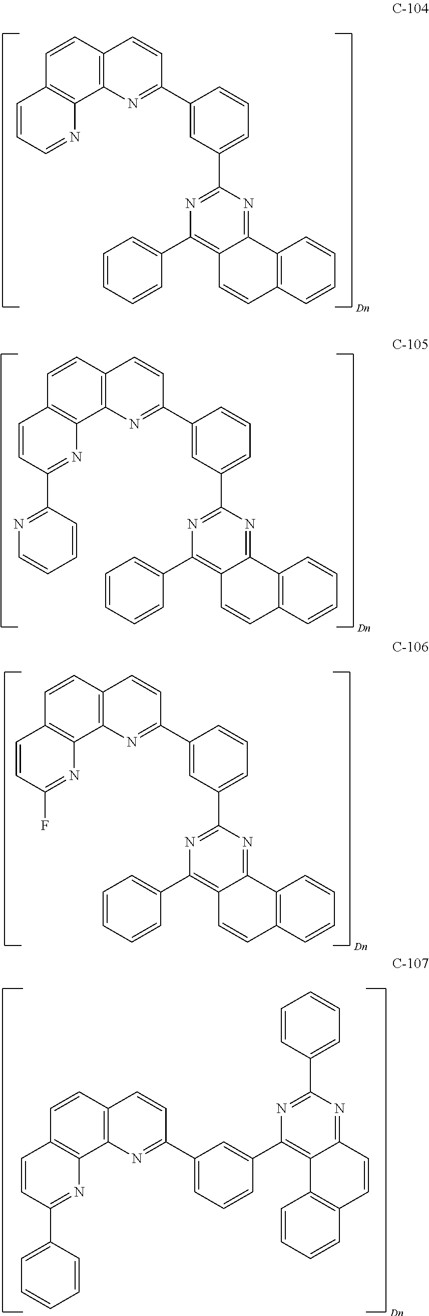

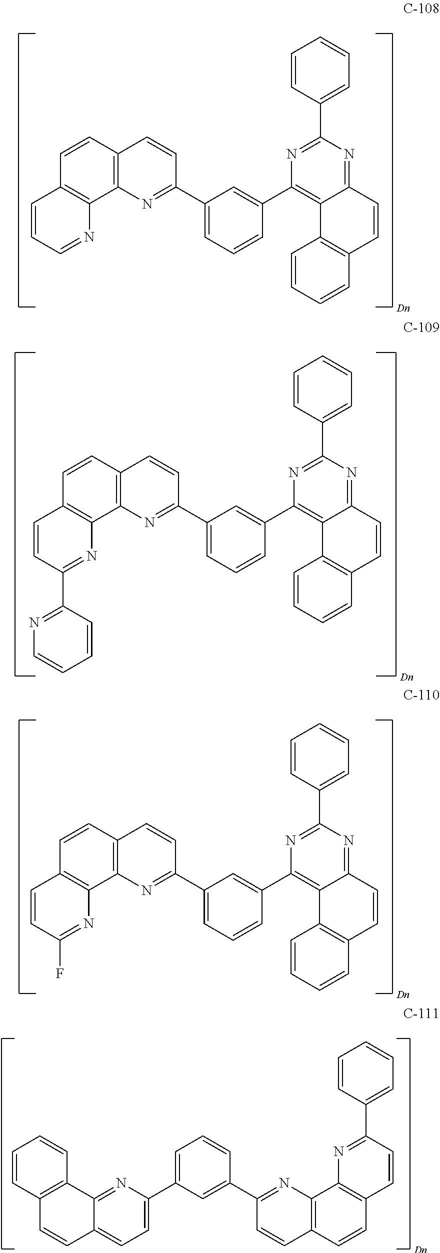

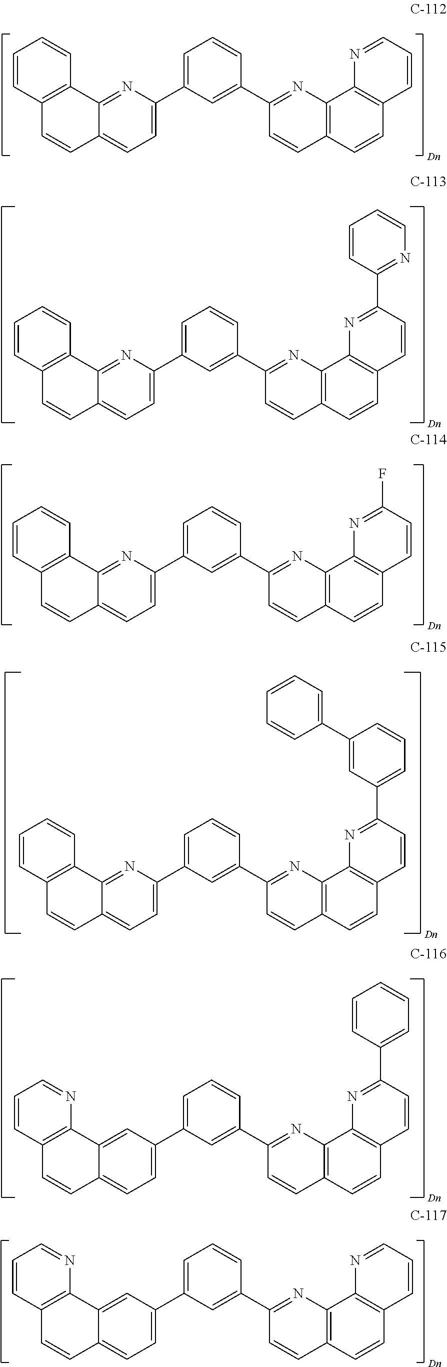

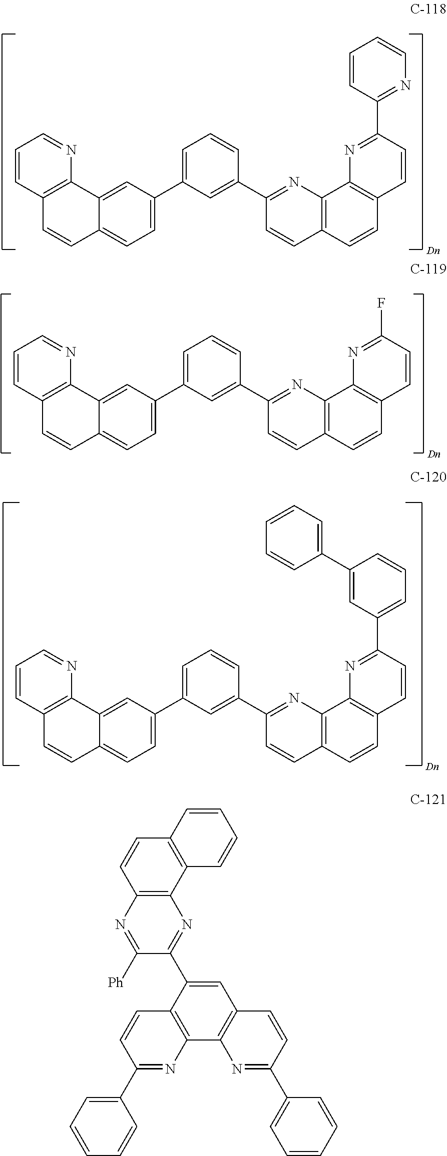

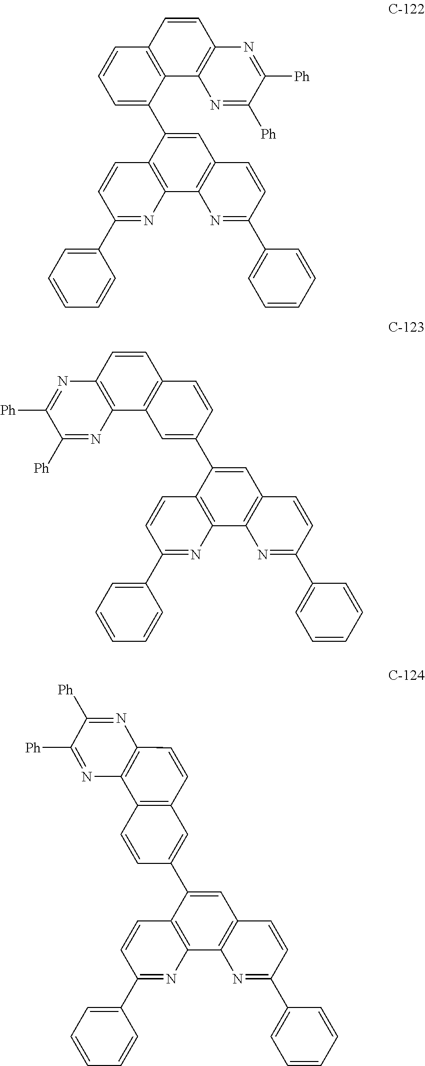 [SMILES: c1ccc(-c2ccc3cc(-c4cccc5ccc6nc(-c7ccccc7)c(-c7ccccc7)nc6c45)c4ccc(-c5ccccc5)nc4c3n2)cc1 ; c1ccc(-c2ccc3cc(-c4ccc5ccc6nc(-c7ccccc7)c(-c7ccccc7)nc6c5c4)c4ccc(-c5ccccc5)nc4c3n2)cc1 ; c1ccc(-c2ccc3cc(-c4ccc5c(ccc6nc(-c7ccccc7)c(-c7ccccc7)nc65)c4)c4ccc(-c5ccccc5)nc4c3n2)cc1]

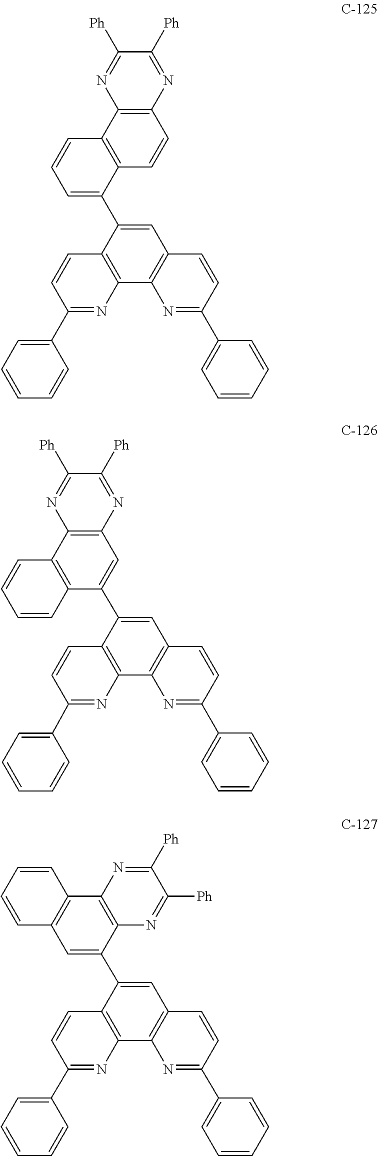

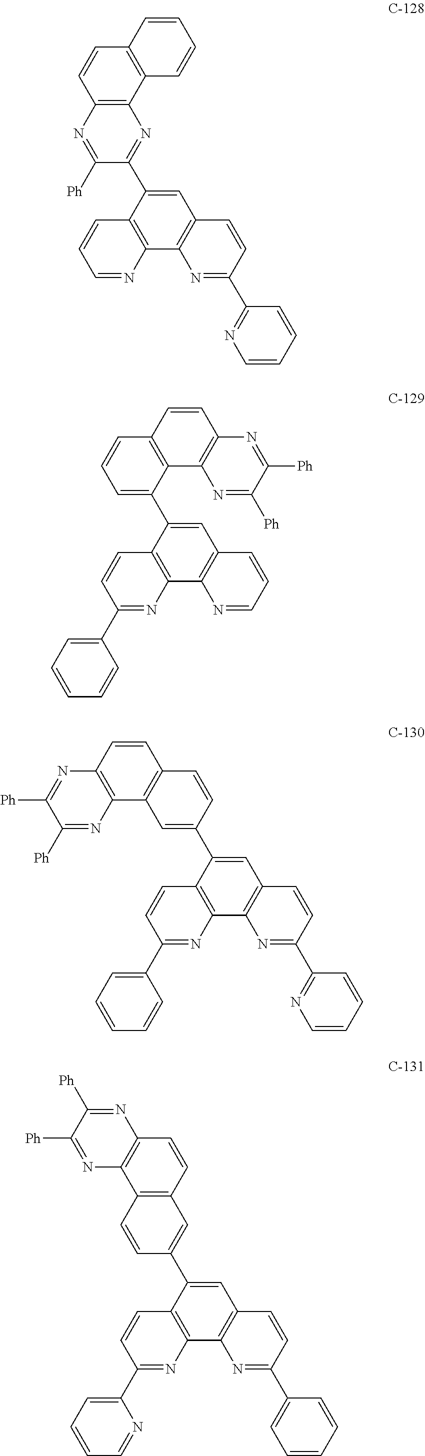

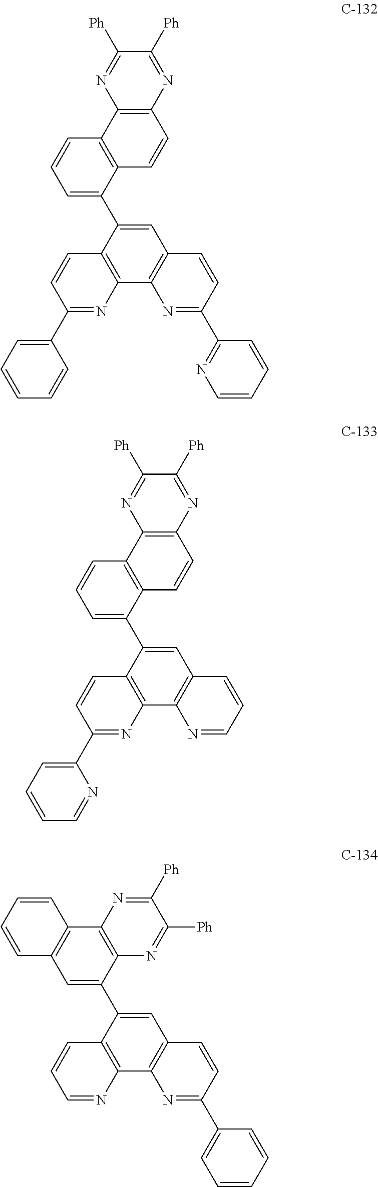 [SMILES: c1ccc(-c2ccc3c(-c4cccc5c4ccc4nc(-c6ccccc6)c(-c6ccccc6)nc45)cc4ccc(-c5ccccn5)nc4c3n2)cc1 ; c1ccc(-c2nc3ccc4c(-c5cc6cccnc6c6nc(-c7ccccn7)ccc56)cccc4c3nc2-c2ccccc2)cc1 ; c1ccc(-c2ccc3cc(-c4cc5ccccc5c5nc(-c6ccccc6)c(-c6ccccc6)nc45)c4cccnc4c3n2)cc1]

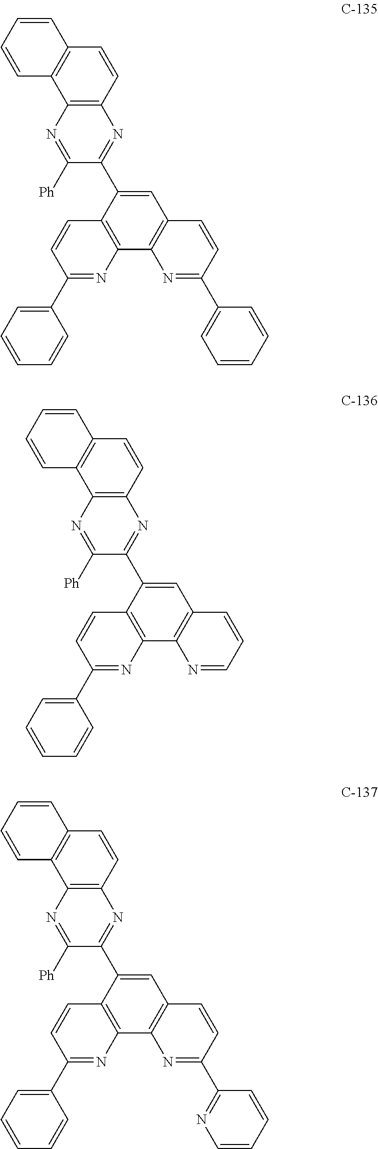

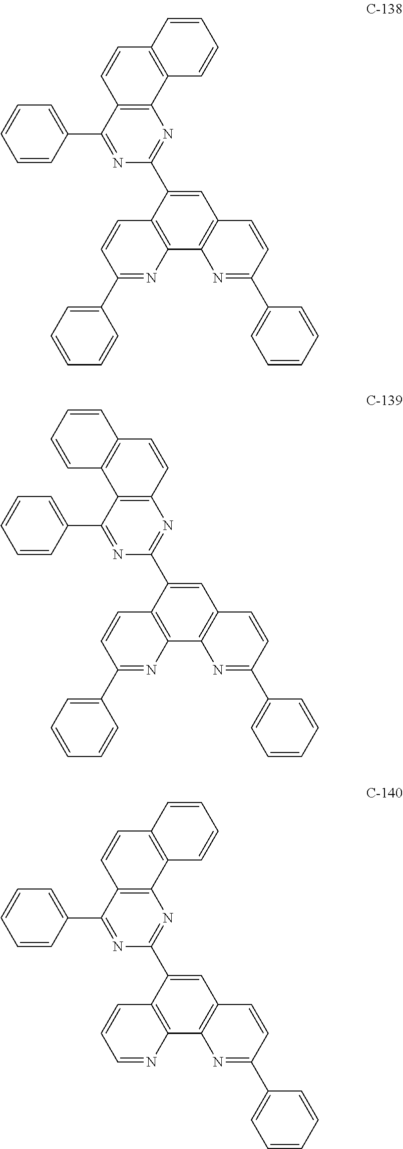

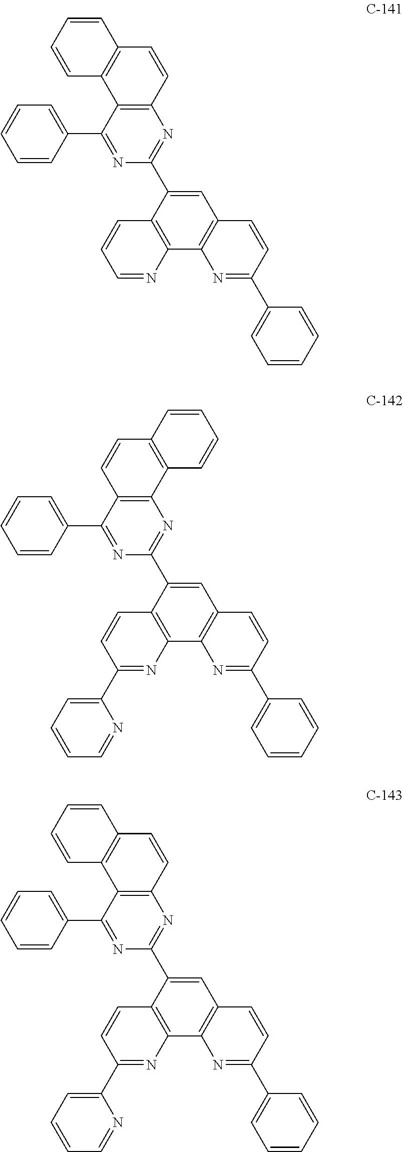 [SMILES: c1ccc(-c2ccc3cc(-c4nc(-c5ccccc5)c5c(ccc6ccccc65)n4)c4cccnc4c3n2)cc1 ; c1ccc(-c2ccc3cc(-c4nc(-c5ccccc5)c5ccc6ccccc6c5n4)c4ccc(-c5ccccn5)nc4c3n2)cc1 ; c1ccc(-c2ccc3cc(-c4nc(-c5ccccc5)c5c(ccc6ccccc65)n4)c4ccc(-c5ccccn5)nc4c3n2)cc1]

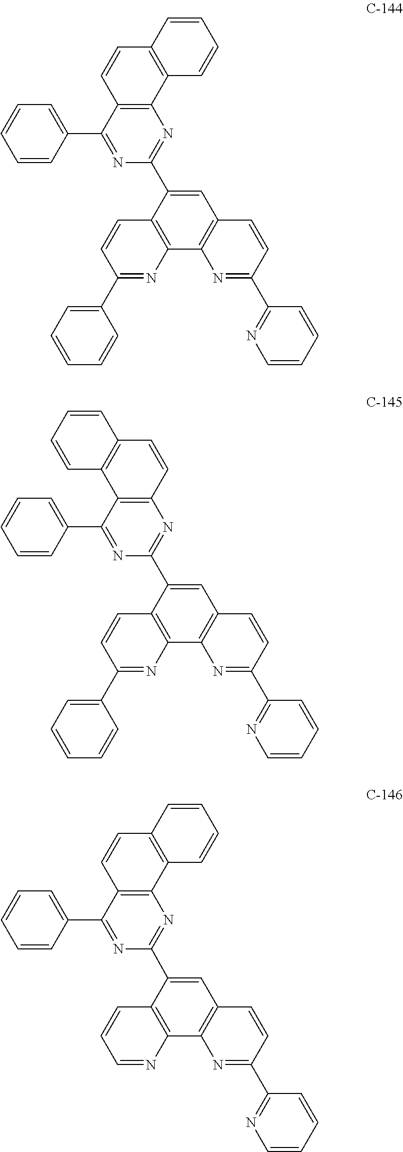 [SMILES: c1ccc(-c2ccc3c(-c4nc(-c5ccccc5)c5ccc6ccccc6c5n4)cc4ccc(-c5ccccn5)nc4c3n2)cc1 ; c1ccc(-c2ccc3c(-c4nc(-c5ccccc5)c5c(ccc6ccccc65)n4)cc4ccc(-c5ccccn5)nc4c3n2)cc1 ; c1ccc(-c2nc(-c3cc4ccc(-c5ccccn5)nc4c4ncccc34)nc3c2ccc2ccccc23)cc1]

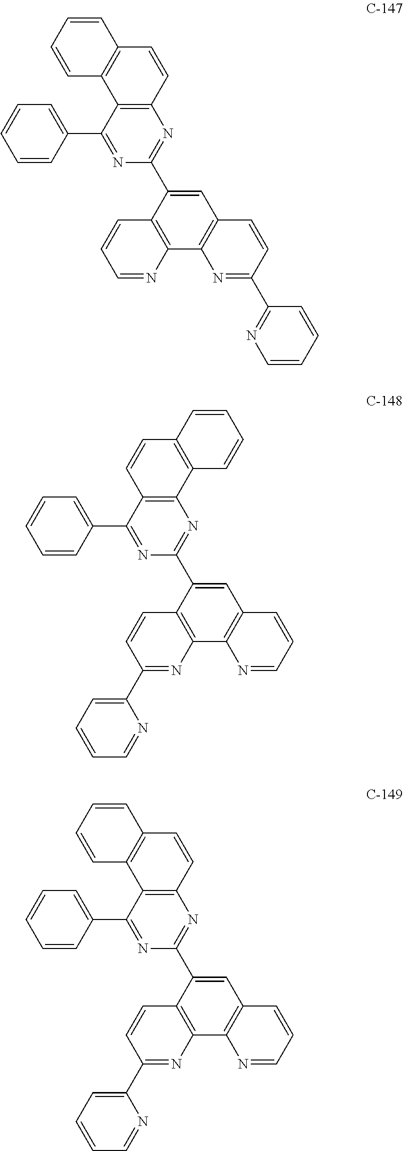 [SMILES: c1ccc(-c2nc(-c3cc4ccc(-c5ccccn5)nc4c4ncccc34)nc3ccc4ccccc4c23)cc1 ; c1ccc(-c2nc(-c3cc4cccnc4c4nc(-c5ccccn5)ccc34)nc3c2ccc2ccccc23)cc1 ; c1ccc(-c2nc(-c3cc4cccnc4c4nc(-c5ccccn5)ccc34)nc3ccc4ccccc4c23)cc1]

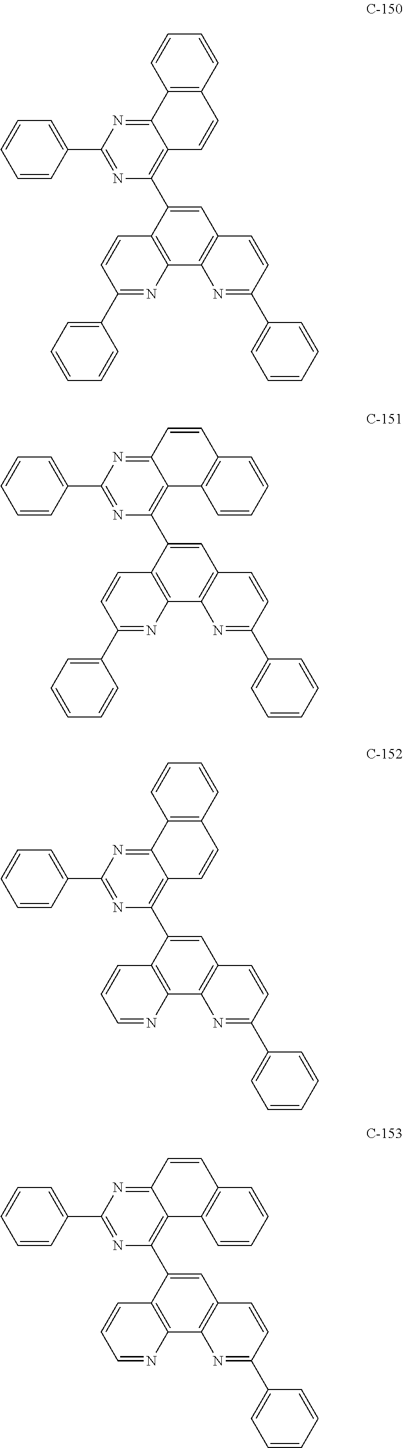 [SMILES: c1ccc(-c2ccc3cc(-c4nc(-c5ccccc5)nc5c4ccc4ccccc45)c4ccc(-c5ccccc5)nc4c3n2)cc1 ; c1ccc(-c2ccc3cc(-c4nc(-c5ccccc5)nc5ccc6ccccc6c45)c4ccc(-c5ccccc5)nc4c3n2)cc1 ; c1ccc(-c2ccc3cc(-c4nc(-c5ccccc5)nc5c4ccc4ccccc45)c4cccnc4c3n2)cc1 ; c1ccc(-c2ccc3cc(-c4nc(-c5ccccc5)nc5ccc6ccccc6c45)c4cccnc4c3n2)cc1]

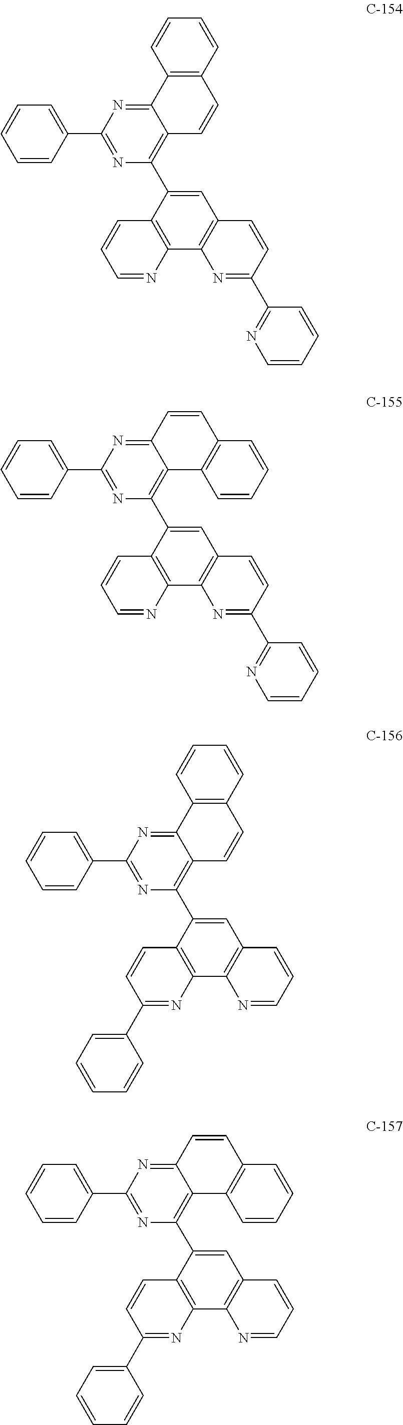 [SMILES: c1ccc(-c2nc(-c3cc4ccc(-c5ccccn5)nc4c4ncccc34)c3ccc4ccccc4c3n2)cc1 ; c1ccc(-c2nc(-c3cc4ccc(-c5ccccn5)nc4c4ncccc34)c3c(ccc4ccccc43)n2)cc1 ; c1ccc(-c2ccc3c(-c4nc(-c5ccccc5)nc5c4ccc4ccccc45)cc4cccnc4c3n2)cc1 ; c1ccc(-c2ccc3c(-c4nc(-c5ccccc5)nc5ccc6ccccc6c45)cc4cccnc4c3n2)cc1]

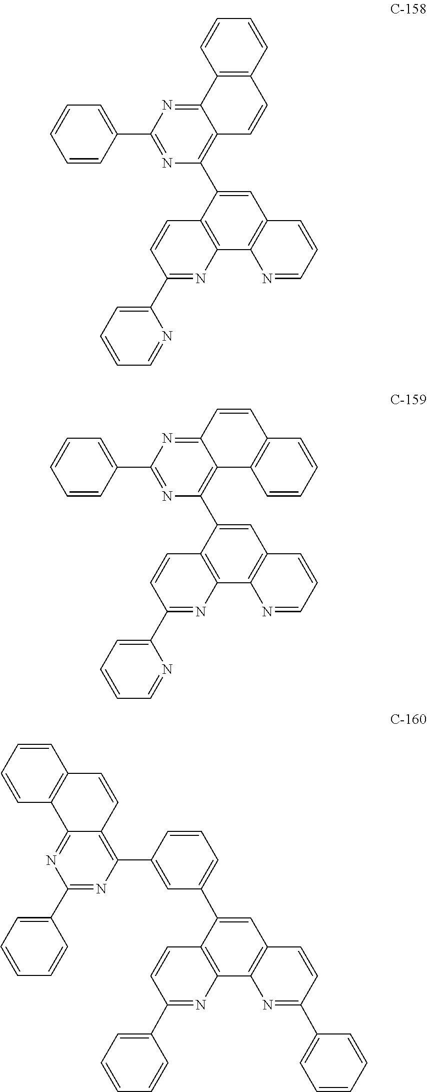 [SMILES: c1ccc(-c2nc(-c3cc4cccnc4c4nc(-c5ccccn5)ccc34)c3ccc4ccccc4c3n2)cc1 ; c1ccc(-c2nc(-c3cc4cccnc4c4nc(-c5ccccn5)ccc34)c3c(ccc4ccccc43)n2)cc1 ; c1ccc(-c2ccc3cc(-c4cccc(-c5nc(-c6ccccc6)nc6c5ccc5ccccc56)c4)c4ccc(-c5ccccc5)nc4c3n2)cc1]

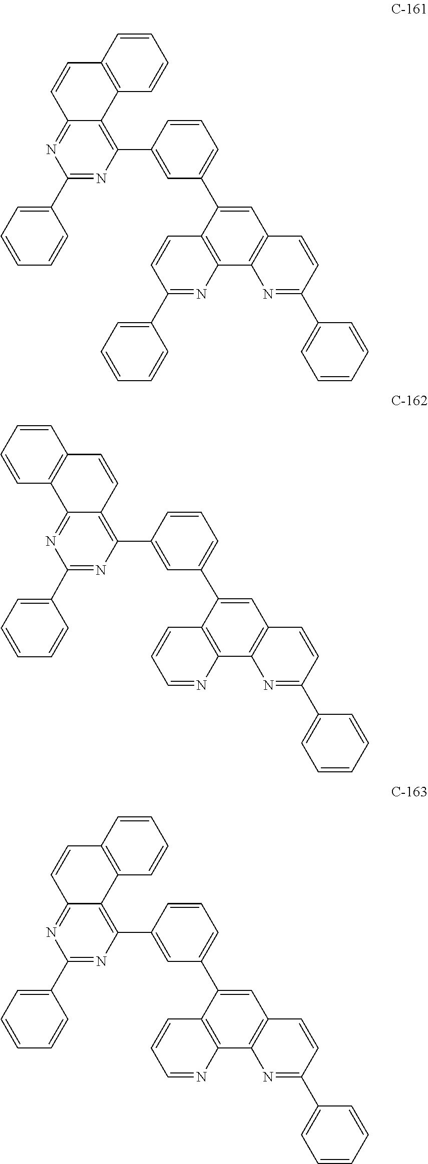 [SMILES: c1ccc(-c2ccc3cc(-c4cccc(-c5nc(-c6ccccc6)nc6ccc7ccccc7c56)c4)c4ccc(-c5ccccc5)nc4c3n2)cc1 ; c1ccc(-c2ccc3cc(-c4cccc(-c5nc(-c6ccccc6)nc6c5ccc5ccccc56)c4)c4cccnc4c3n2)cc1 ; c1ccc(-c2ccc3cc(-c4cccc(-c5nc(-c6ccccc6)nc6ccc7ccccc7c56)c4)c4cccnc4c3n2)cc1]

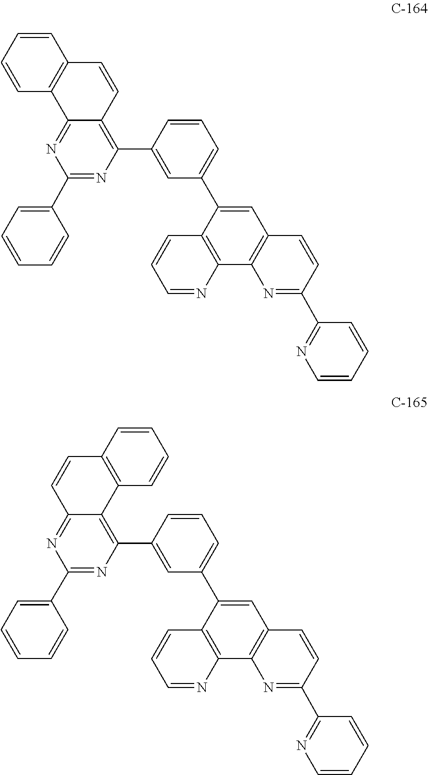 [SMILES: c1ccc(-c2nc(-c3cccc(-c4cc5ccc(-c6ccccn6)nc5c5ncccc45)c3)c3ccc4ccccc4c3n2)cc1 ; c1ccc(-c2nc(-c3cccc(-c4cc5ccc(-c6ccccn6)nc5c5ncccc45)c3)c3c(ccc4ccccc43)n2)cc1]

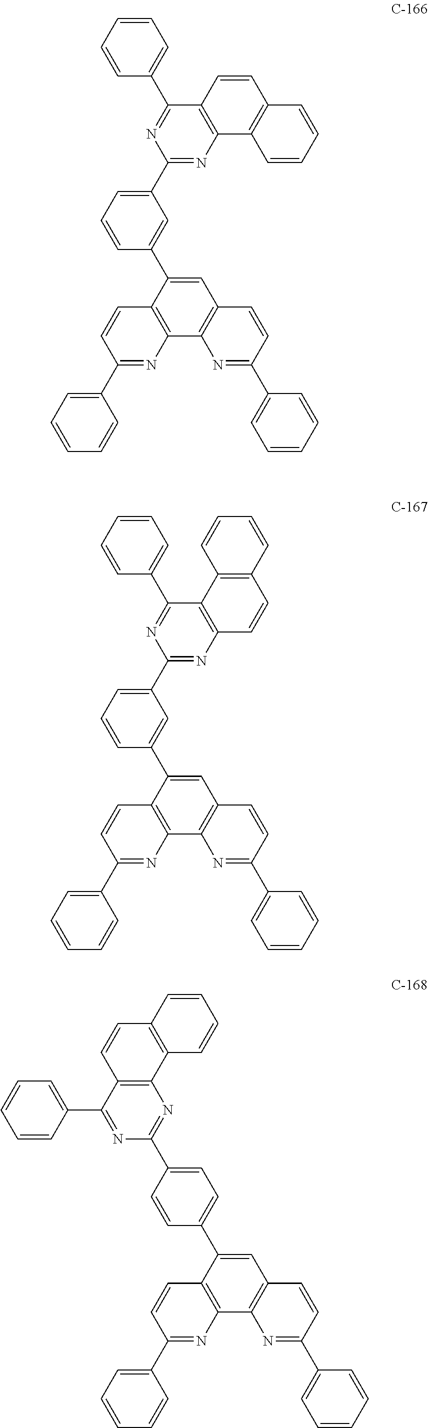 [SMILES: c1ccc(-c2ccc3cc(-c4cccc(-c5nc(-c6ccccc6)c6ccc7ccccc7c6n5)c4)c4ccc(-c5ccccc5)nc4c3n2)cc1 ; c1ccc(-c2ccc3cc(-c4cccc(-c5nc(-c6ccccc6)c6c(ccc7ccccc76)n5)c4)c4ccc(-c5ccccc5)nc4c3n2)cc1 ; c1ccc(-c2ccc3cc(-c4ccc(-c5nc(-c6ccccc6)c6ccc7ccccc7c6n5)cc4)c4ccc(-c5ccccc5)nc4c3n2)cc1]

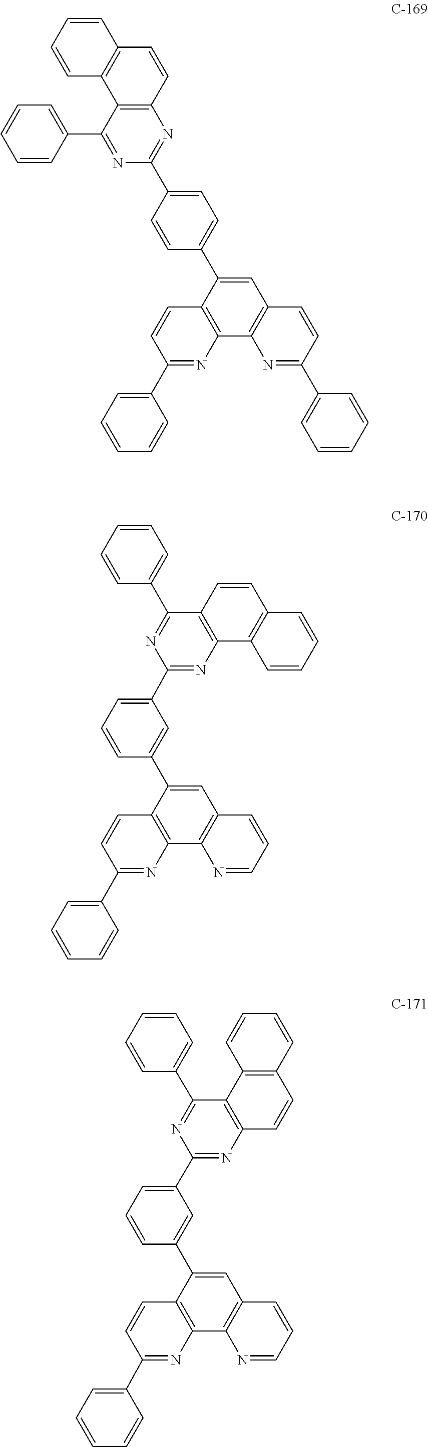 [SMILES: c1ccc(-c2ccc3cc(-c4ccc(-c5nc(-c6ccccc6)c6c(ccc7ccccc76)n5)cc4)c4ccc(-c5ccccc5)nc4c3n2)cc1 ; c1ccc(-c2ccc3c(-c4cccc(-c5nc(-c6ccccc6)c6ccc7ccccc7c6n5)c4)cc4cccnc4c3n2)cc1 ; c1ccc(-c2ccc3c(-c4cccc(-c5nc(-c6ccccc6)c6c(ccc7ccccc76)n5)c4)cc4cccnc4c3n2)cc1]

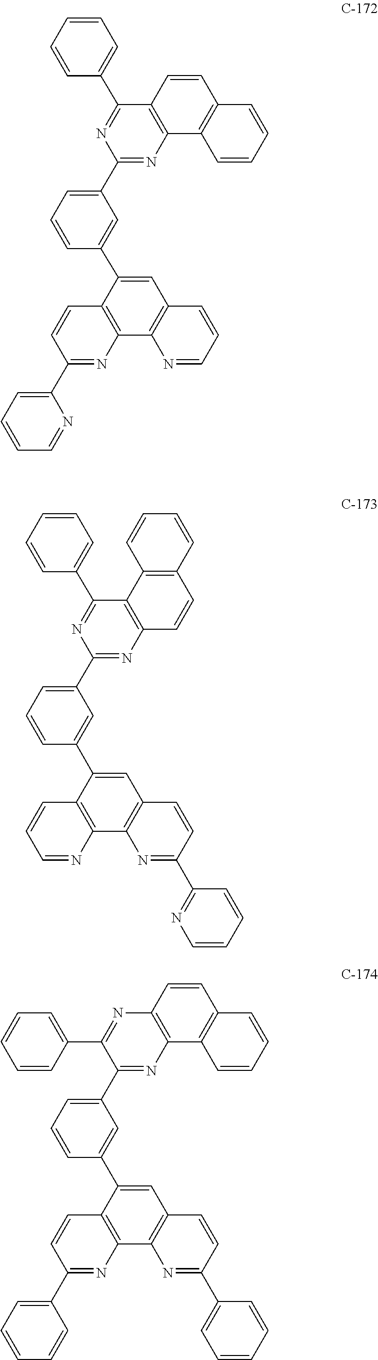

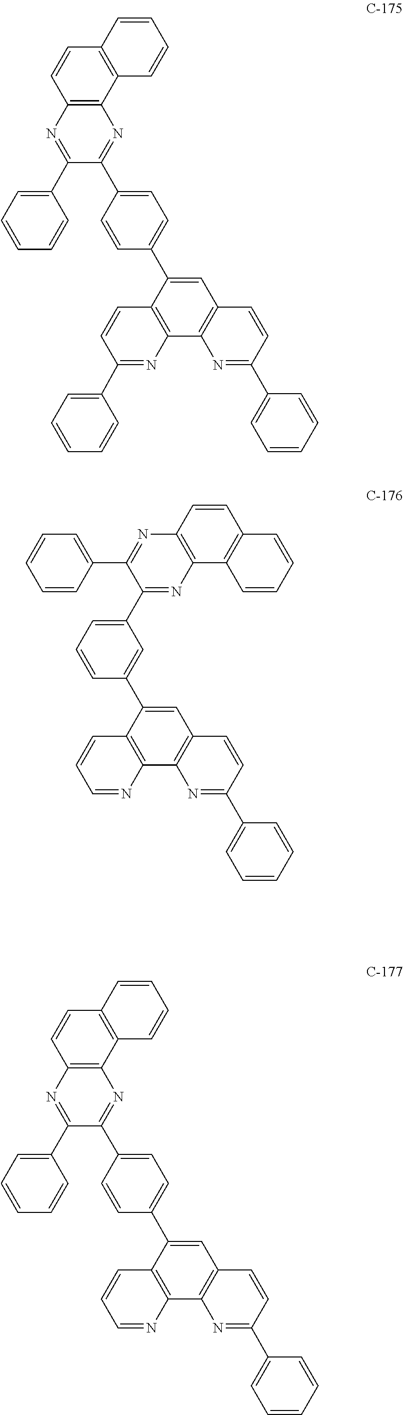 [SMILES: c1ccc(-c2ccc3cc(-c4ccc(-c5nc6c(ccc7ccccc76)nc5-c5ccccc5)cc4)c4ccc(-c5ccccc5)nc4c3n2)cc1 ; c1ccc(-c2ccc3cc(-c4cccc(-c5nc6c(ccc7ccccc76)nc5-c5ccccc5)c4)c4cccnc4c3n2)cc1 ; c1ccc(-c2ccc3cc(-c4ccc(-c5nc6c(ccc7ccccc76)nc5-c5ccccc5)cc4)c4cccnc4c3n2)cc1]

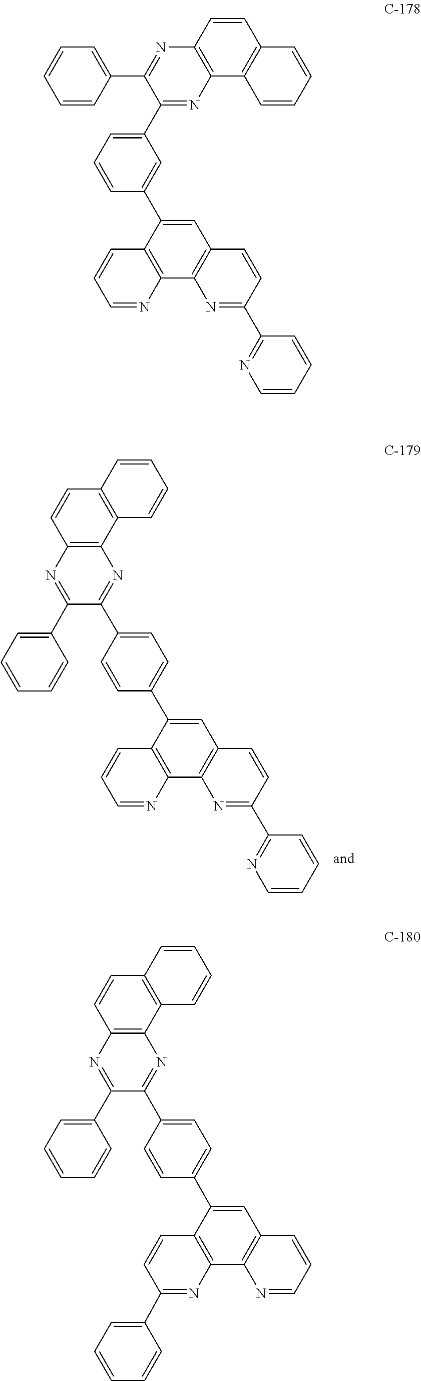 [SMILES: c1ccc(-c2nc3ccc4ccccc4c3nc2-c2cccc(-c3cc4ccc(-c5ccccn5)nc4c4ncccc34)c2)cc1 ; c1ccc(-c2nc3ccc4ccccc4c3nc2-c2ccc(-c3cc4ccc(-c5ccccn5)nc4c4ncccc34)cc2)cc1 ; c1ccc(-c2ccc3c(-c4ccc(-c5nc6c(ccc7ccccc76)nc5-c5ccccc5)cc4)cc4cccnc4c3n2)cc1]

In the compounds above, Dₙ means that n number of hydrogens are replaced with deuterium, and n represents an integer from 1 to the maximum number of hydrogens in the compound. Specifically, n is an integer with a minimum of 1 and a maximum of the number of hydrogens in the compound.

According to one embodiment of the present disclosure, in a compound represented by Formula 1, when deuterium is included, the deuterium substitution rate is preferably about 100% or less of the total number of hydrogens, more preferably 95% or less, still more preferably 90% or less, and even more preferably 85% or less. The compound of Formula 1 substituted with the above deuterium substitution rate can increase the stability of the compound by increasing the bond dissociation energy according to deuteration, and an organic electroluminescent device including the compound can exhibit improved luminescence characteristics.

The compounds represented by Formula 1 may be produced by synthetic methods known to one skilled in the art. For example, the compounds of the present disclosure may be synthesized by referring to the following Reaction Scheme 1, but are not limited thereto.

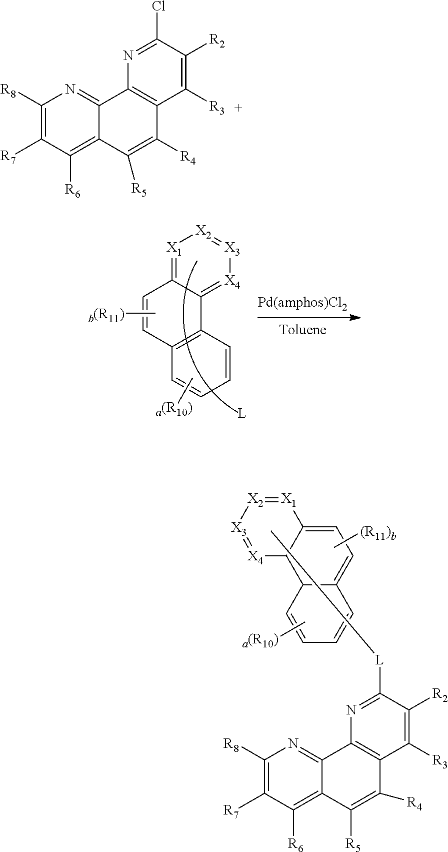

In Reaction Scheme 1, R₂ to R₈, R₁₀, R₁₁, X₁ to X₄, L, a, and b are as defined in Formula 1, respectively.

The present invention provides an organic electroluminescent material, an N-type charge-generating material, or an organic electroluminescent device comprising the above compound.

An organic electroluminescent device according to one embodiment of the present disclosure comprises a first electrode; a second electrode; a plurality of light-emitting units positioned between the first electrode and the second electrode; and at least one charge generation layer positioned between adjacent light-emitting units among the plurality of light-emitting units, wherein the charge generation layer may comprise a compound represented by Formula 1.

An organic electroluminescent device according to one embodiment of the present disclosure may be an organic electroluminescent device having a tandem structure. In the case of the tandem organic electroluminescent device according to one embodiment, a single light-emitting unit (light-emitting part) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer stacked between the first and second electrodes and which emits light in a specific wavelength range. It may include a plurality of light-emitting units, and each of the light-emitting units may include a hole transport zone, a light-emitting layer, and an electron transport zone, the hole transport zone may include a hole injection layer and a hole transport layer, and the electron transport zone may include an electron transport layer and an electron injection layer. According to one embodiment of the present disclosure, three or more light-emitting layers may be included in the light-emitting unit. A plurality of light-emitting units may emit the same color or different colors. In addition, one light-emitting unit may include one or more light-emitting layers, and the plurality of light-emitting layers may be light-emitting layers of the same or different colors. It may include one or more charge generation layers located between each of the light-emitting units.

The charge generation layer refers to the layer in which holes and electrons are generated when voltage is applied. When there are three or more light-emitting units, a charge generation layer may be located between each light-emitting unit. The plurality of charge generation layers may be the same as or different from one another. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of an anode and a cathode without a separate internal electrode located between the stacks. The charge generation layer may be composed of an N-type charge generation layer and a P-type charge generation layer. The N-type charge generation layer may comprise a compound represented by Formula 1 of the present disclosure. The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. In addition, commonly used materials may be used as the P-type dopant and host materials used in the P-type doped organic material.

The organic electroluminescent device according to another embodiment of the present disclosure comprises a first electrode; a second electrode; and at least one organic layer between the first electrode and the second electrode, wherein the organic layer comprises a hole transport layer, a light-emitting layer, a hole auxiliary layer, an electron-blocking layer, a charge generation layer, and a light-emitting auxiliary layer. According to one embodiment of the present disclosure, at least one layer among the hole transport layer, the light-emitting layer, the hole auxiliary layer, the electron-blocking layer, the charge generation layer, and the light-emitting auxiliary layer may comprise a compound represented by Formula 1. For example, the charge generation layer may comprise a compound represented by Formula 1. According to one embodiment of the present disclosure, the organic electroluminescent material of the present disclosure comprises at least one compound among Compounds C-1 to C-120, and the organic electroluminescent material can be comprised in the same organic layer, for example, a charge generation layer.

The organic layer may further comprise at least one layer selected from a hole injection layer, an electron transport layer, an electron injection layer, an interlayer, a hole-blocking layer, and an electron buffer layer, in addition to a hole transport layer, a light-emitting layer, a hole auxiliary layer, an electron-blocking layer, a charge generation layer, and a light-emitting auxiliary layer. The organic layer may additionally comprise an amine-based compound and/or an azine-based compound in addition to the compound of the present disclosure. Specifically, the hole injection layer, the hole transport layer, the hole auxiliary layer, the light-emitting layer, the light-emitting auxiliary layer, or the electron-blocking layer may comprise an amine-based compound, for example, an arylamine-based compound, a styrylarylamine-based compound, etc., as a hole injection material, a hole transport material, a hole auxiliary material, a light-emitting material, a light-emitting auxiliary material, and an electron-blocking material. In addition, the electron transport layer, the electron injection layer, the electron buffer layer and the hole-blocking layer may comprise an azine-based compound as an electron transport material, an electron injection material, an electron buffer material, and a hole-blocking material. In addition, the organic material layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

The compound according to one embodiment of the present disclosure may be used as a light-emitting material for a white organic light-emitting device. The white organic light-emitting device has been suggested to have various structures such as a side-by-side arrangement method, a stacking arrangement method, or a color conversion material (CCM) method, etc., according to the arrangement of R (red), G (green) or YG (yellowish green), and B (blue) light-emitting units. In addition, the organic electroluminescent compound according to one embodiment of the present disclosure may also be used in an organic electroluminescent device comprising a quantum dot (QD).

One of the first and second electrodes may be an anode, and the other may be a cathode. The first electrode and the second electrode may each be formed with a transmissive conductive material, a transflective conductive material, or a reflective conductive material. The organic electroluminescent device may be a top emission type, a bottom emission type, or both-sides emission type depending on the type of the material forming the first electrode and the second electrode.

The light-emitting layer may include one or more hosts and one or more dopants. The dopant comprised in the organic electroluminescent device of the present disclosure may be at least one phosphorescent or fluorescent dopant, and is preferably a phosphorescent dopant.

A hole injection layer, a hole transport layer, an electron-blocking layer, or a combination thereof may be used between an anode and a light-emitting layer. The hole injection layer may be multi-layered in order to lower the hole injection barrier (or hole injection voltage) from the anode to the hole transport layer or the electron-blocking layer, wherein two compounds may be simultaneously used in each of the multi-layers. In addition, the hole injection layer may be further doped with a p-dopant. The electron-blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron transport layer may also be multi-layers, wherein each of the multi-layers may use a plurality of compounds.

An electron buffer layer, a hole-blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layers, wherein each layer may use a plurality of compounds. Also, the electron injection layer may be doped as an n-dopant.

A light-emitting auxiliary layer may be a layer placed between an anode and a light-emitting layer, or between a cathode and a light-emitting layer. When placed between the anode and the light-emitting layer, the light-emitting auxiliary layer may be used to facilitate hole injection and/or hole transport or to block the overflow of electrons. When placed between the cathode and the light-emitting layer, the light-emitting auxiliary layer may be used to facilitate electron injection and/or electron transport or to block the overflow of holes. In addition, the hole auxiliary layer may be placed between the hole transport layer (or hole injection layer) and the light-emitting layer, and may exhibit an effect of facilitating or blocking the hole transport rate (or hole injection rate), and accordingly may adjust the charge balance. When an organic electroluminescent device includes two or more hole transport layers, the hole transport layer, which is further included, may be used as a hole auxiliary layer or an electron-blocking layer. The light-emitting auxiliary layer, the hole auxiliary layer, or the electron-blocking layer may have an effect of improving the efficiency and/or lifetime of the organic electroluminescent device.

In the organic electroluminescent device of the present disclosure, it is preferable to dispose at least one layer selected from a chalcogenide layer, a metal halide layer, and a metal oxide layer (hereinafter referred to as a “surface layer”) on at least one inner surface of a pair of electrodes. Specifically, a chalcogenide (including an oxide) layer of silicon and aluminum is preferably placed on an anode surface of an electroluminescent medium layer side, and a metal halide layer or a metal oxide layer is preferably placed on a cathode surface of an electroluminescent medium layer side.

Driving stabilization of the organic electroluminescent device can be obtained by the surface layer. Preferred examples of the chalcogenide include SiO_{X} (1≤X≤2), AlO_{X} (1≤X≤1.5), SiON, SiAlON, etc., preferred examples of the metal halide include LiF, MgF₂, CaF₂, a rare earth metal fluoride, etc., and preferred examples of the metal oxide include Cs₂O, Li₂O, MgO, SrO, BaO, CaO, etc.

In addition, in an organic electroluminescent device of the present disclosure, a mixed region of an electron transport compound and a reductive dopant, or a mixed region of a hole transport compound and an oxidative dopant, may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferred oxidative dopants include various Lewis acids and acceptor compounds, and preferred reductive dopants include alkali metals, alkali metal compounds, alkaline earth metals, rare earth metals, and mixtures thereof. In addition, an organic electroluminescent device having at least two light-emitting layers and emitting white light may be manufactured by using the reductive dopant layer as a charge-generating layer.

According to one embodiment, the present disclosure can provide a display device comprising a compound represented by Formula 1. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of the organic electroluminescent compounds according to the present disclosure, the physical properties thereof, and the driving voltage, current efficiency, and lifetime properties of the OLED according to the present disclosure will be explained in detail. However, the following examples only describe the properties of the compound and the OLED according to the present disclosure, but the present disclosure is not limited to the following examples.

### Example 1: Preparation of Compound C-5

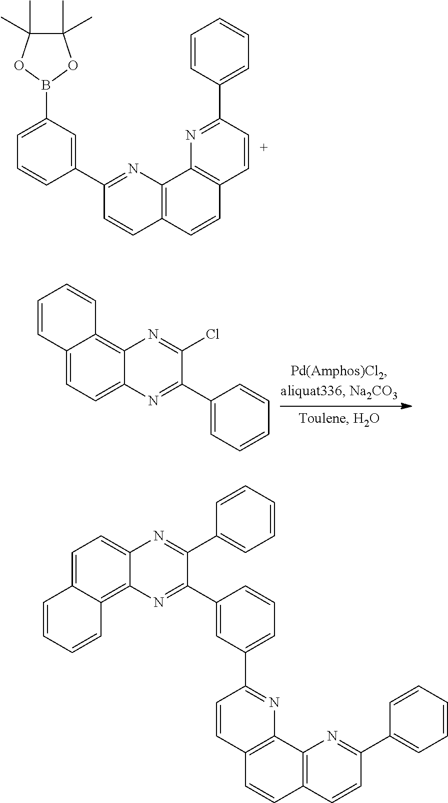

2-Phenyl-9-(3-(4,4,5,5-tetramethyl-1,3,2-dioxaborolan-2-yl)phenyl)-1,10-phenanthroline (20.0 g, 43.6 mmol), 2-chloro-3-phenylbenzo[f]quinoxaline (12.7 g, 43.6 mmol), Pd(Amphos)Cl₂ (2.2 g, 3.1 mmol), aliquot 336 (1.8 g, 4.4 mmol), and Na₂CO₃ (9.2 g, 87 mmol) were dissolved in 220 mL of toluene and 75 mL of H₂O, and the mixture was then stirred under reflux at 130° C. for 5 hours. After completion of the reaction, the mixture was cooled to room temperature, and the organic layer was separated. Thereafter, the residue was separated using column chromatography to obtain Compound C-5 (12.5 g, yield: 49%).

| | MW | M.P. |
|---|---|---|
| C-5 | 586.2 | 259° C. |

### Example 2: Preparation of Compound C-2

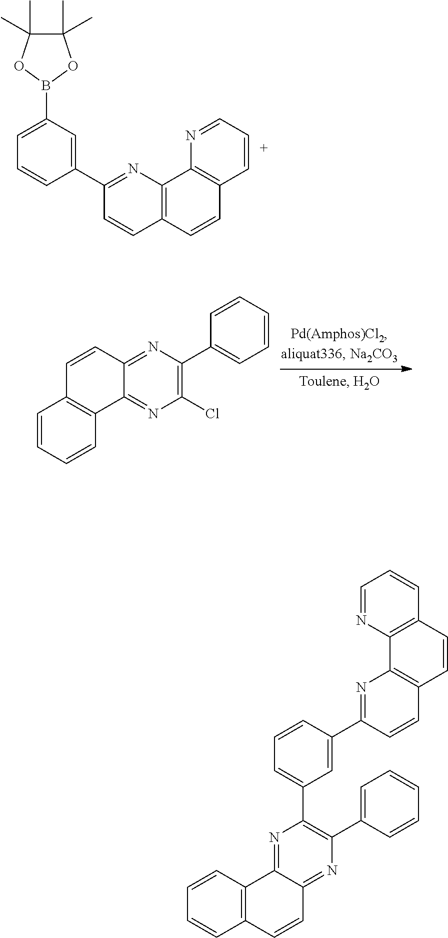 [SMILES: CC1(C)OB(c2cccc(-c3ccc4ccc5cccnc5c4n3)c2)OC1(C)C ; Clc1nc2c(ccc3ccccc32)nc1-c1ccccc1 ; c1ccc(-c2nc3ccc4ccccc4c3nc2-c2cccc(-c3ccc4ccc5cccnc5c4n3)c2)cc1]

2-(3-(4,4,5,5-Tetramethyl-1,3,2-dioxaborolan-2-yl)phenyl)-1,10-phenanthroline (10.0 g, 26.2 mmol), 2-chloro-3-phenylbenzo[ ]quinoxaline (9.1 g, 31.4 mmol), Pd(Amphos)Cl₂ (1.3 g, 1.8 mmol), aliquot 336 (1.1 g, 2.6 mmol), and Na₂CO₃ (5.5 g, 52.3 mmol) were dissolved in 130 mL of toluene and 45 mL of H₂O, and the mixture was then stirred under reflux at 130° C. for 3 hours. After completion of the reaction, the mixture was cooled to room temperature, and the organic layer was separated. Thereafter, the residue was separated using column chromatography to obtain Compound C-2 (7.3 g, yield: 54%).

| | MW | M.P. |
|---|---|---|
| C-2 | 510.6 | 280° C. |

### Example 3: Preparation of Compound C-9

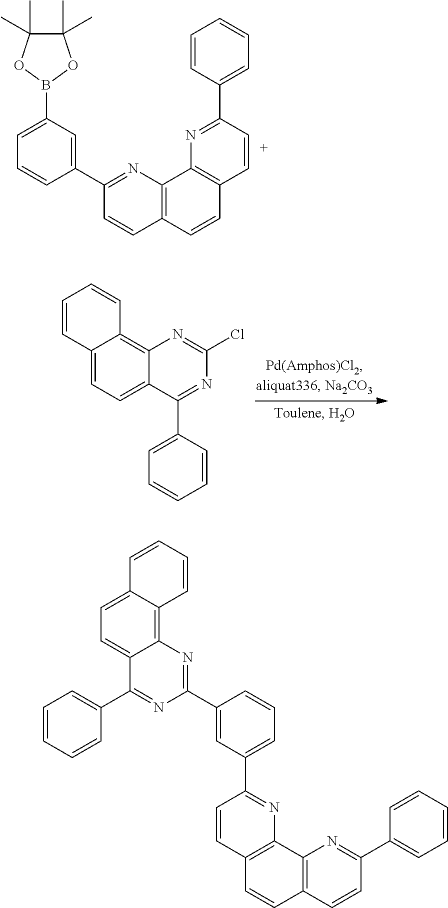

2-Phenyl-9-(3-(4,4,5,5-tetramethyl-1,3,2-dioxaborolan-2-yl)phenyl)-1,10-phenanthroline (10.0 g, 21.8 mmol), 2-chloro-4-phenylbenzo[h]quinazoline (7.6 g, 26 mmol), Pd(Amphos)Cl₂ (1.1 g, 1.5 mmol), aliquot 336 (0.9 g, 2.2 mmol), and Na₂CO₃ (4.6 g, 44 mmol) were dissolved in 110 mL of toluene and 36 mL of H₂O, and the mixture was then stirred under reflux at 130° C. for 5 hours. After completion of the reaction, the mixture was cooled to room temperature, and the organic layer was separated. Thereafter, the residue was separated using column chromatography to obtain Compound C-9 (7.1 g, yield: 55%).

| | MW | M.P. |
|---|---|---|
| C-9 | 586.7 | 285° C. |

### Example 4: Preparation of Compound C-10

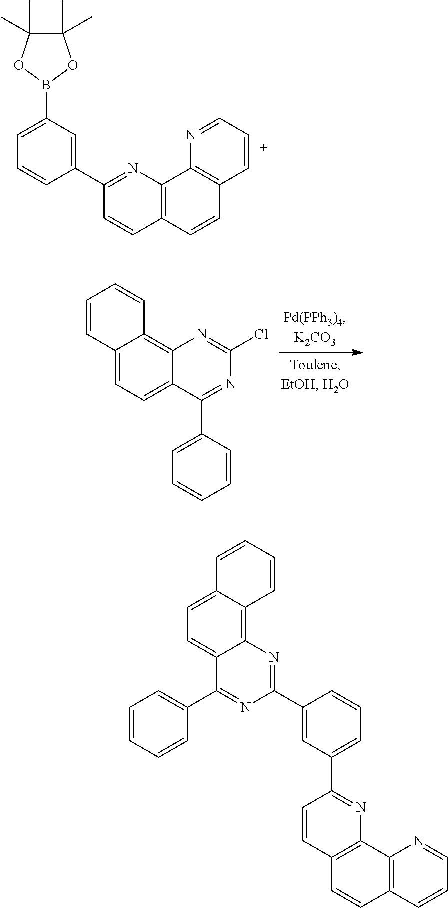 [SMILES: CC1(C)OB(c2cccc(-c3ccc4ccc5cccnc5c4n3)c2)OC1(C)C ; Clc1nc(-c2ccccc2)c2ccc3ccccc3c2n1 ; c1ccc(-c2nc(-c3cccc(-c4ccc5ccc6cccnc6c5n4)c3)nc3c2ccc2ccccc23)cc1]

2-(3-(4,4,5,5-Tetramethyl-1,3,2-dioxaborolan-2-yl)phenyl)-1,10-phenanthroline (19.1 g, 50 mmol), 2-chloro-4-phenylbenzo[h]quinazoline (14.5 g, 50 mmol), Pd(PPh₃)₄ (2.9 g, 2.5 mmol), and K₂CO₃ (17.3 g, 125 mmol) were dissolved in 200 mL of toluene, 50 mL of EtOH, and 50 mL of H₂O, and the mixture was then stirred under reflux at 130° C. for 18 hours. After completion of the reaction, the mixture was cooled to room temperature, and the organic layer was separated. Thereafter, the residue was separated using column chromatography to obtain Compound C-10 (22.0 g, yield: 86%)

| | MW | M.P. |
|---|---|---|
| C-10 | 510.60 | 256° C. |

### Device Examples 1 to 4: Producing OLEDs Comprising the Compound According to the Present Disclosure in an N-Type Charge-Generating Layer

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1 was introduced into a cell of the vacuum vapor deposition apparatus, and Compound HT-3 was introduced into another cell. The two materials were evaporated at different rates, and Compound HI-1 was deposited in a doping amount of 3 wt % based to the total amount of Compound HI-1 and Compound HT-3 to form a hole injection layer with a thickness of 5 nm. Subsequently, Compound HT-3 was deposited on the hole injection layer to form a first hole transport layer with a thickness of 30 nm. Compound HT-4 was then introduced into another cell of the vacuum vapor deposition apparatus and was evaporated by applying an electric current to the cell, thereby depositing a second hole transport layer with a thickness of 5 nm on the first hole transport layer. After forming the hole injection layer and the hole transport layers, a first light-emitting layer was deposited thereon as follows: Compound H-1 was introduced into a cell of the vacuum vapor deposition apparatus as a host, and Compound D-1 was introduced into another cell as a dopant. The two materials were evaporated at different rates, and the dopant was deposited in a doping amount of 2 wt % based on the total amount of the host and the dopant to form a first light-emitting layer with a thickness of 20 nm on the second hole transport layer. Subsequently, Compound ET-1 was deposited to a thickness of 5 nm as a first hole-blocking layer material on the first light-emitting layer. Compound ET-2 was then deposited as an electron transport layer material with a thickness of 10 nm to form a first electron transport layer. Thereafter, the N-type charge generation layer was formed to a thickness of 4 nm by depositing 0.5 wt % of Li (lithium) on the compound of the N-type charge generation layer described in Table 1. Subsequently, Compound HI-1 was deposited in a doping amount of 6 wt % based to the total amount of Compound HI-1 and Compound HT-3 to form a P-type charge generation layer with a thickness of 10 nm. After Compound HT-3 was deposited to a thickness of 30 nm to form a third hole transport layer, Compound HT-4 was deposited to a thickness of 5 nm to form a fourth hole transport layer. Subsequently, a second light-emitting layer was deposited thereon as follows: Compound H-1 was introduced into a cell of the vacuum vapor deposition apparatus as a host, and Compound D-1 was introduced into another cell as a dopant. The two materials were evaporated at different rates, and the dopant was deposited in a doping amount of 2 wt % based on the total amount of the host and the dopant to form a second light-emitting layer with a thickness of 20 nm on the fourth hole transport layer. Compound ET-1 was deposited to a thickness of 5 nm as a second hole-blocking layer material on the second light-emitting layer, Compounds ET-2 and EI-1 were introduced into two cells of the vacuum deposition device as second electron transport layer materials, respectively, and the two materials were deposited at a weight ratio of 2:1 to a thickness of 25 nm. After Yb was deposited as an electron injection layer with a thickness of 1 nm on the second electron transport layer, an Al cathode was deposited with a thickness of 80 nm on the electron injection layer using another vacuum vapor deposition apparatus to manufacture an OLED. For each material, each compound was purified by vacuum sublimation under 10⁻⁶ Torr and used.

### Comparative Examples 1 and 2: Producing OLEDs Comprising the Comparative Compound in an N-Type Charge-Generating Layer

An OLED was produced in the same manner as in Device Example 1, except that the respective compounds of the N-type charge generation layer described in Table 1 below were used.

The driving voltage, current efficiency, the time taken for luminance to reduce from 100% to 95% at a luminance of 1,000 nit when checking lifetime with 2× accelerated aging (lifetime: T₉₅), and progressive driving voltage change after 10 hours (ΔV) of the OLED produced in Device Examples 1 to 4 and Comparative Examples 1 and 2 as described above were measured, and the results thereof are shown in the following Table 1.

TABLE 1

| | N-type | | | | |
|---|---|---|---|---|---|
| | charge | Driving | Current | | |
| | generation | voltage | efficiency | Lifetime T₉₅ | |
| | layer | (V) | (cd/A) | [hr] | ΔV [%] |
| Device | C-5 | 6.5 | 7.7 | 180.7 | 100.9 |
| Example 1 | | | | | |
| Device | C-2 | 6.4 | 7.7 | 177.8 | 100.8 |
| Example 2 | | | | | |
| Device | C-9 | 6.5 | 7.7 | 180.7 | 100.7 |
| Example 3 | | | | | |
| Device | C-10 | 6.5 | 7.7 | 181.6 | 100.8 |
| Example 4 | | | | | |
| Comparative | T-1 | 6.6 | 7.4 | 152 | 101.1 |
| Example 1 | | | | | |
| Comparative | T-2 | 6.5 | 7.6 | 151.4 | 100.9 |
| Example 2 | | | | | |

From Table 1 above, it can be confirmed that the OLEDs comprising the compound according to the present disclosure in an N-type charge generation layer have an effect equivalent to or greater than that of the OLEDs comprising a conventional compound in terms of driving voltage and/or current efficiency, while exhibiting high lifetime characteristics and/or low progressive driving voltage variation. As the change in driving voltage became greater, the increase in driving voltage over time likewise became greater, which may result in increased power consumption, overheating of the component, and shortened lifespan of the component.

The compounds used in Device Examples and Comparative Examples are shown in Table 2 below.

TABLE 2

| | |
|---|---|
| Hole Injection Layer/Hole Transport Layer | 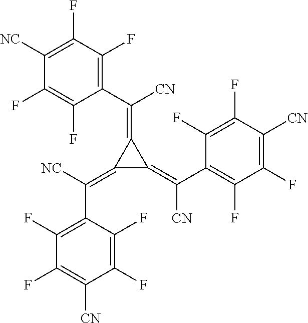 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 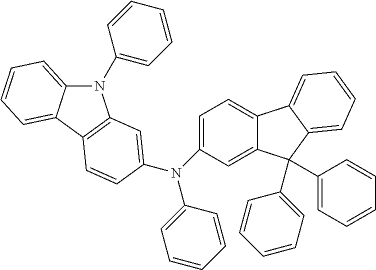 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-3 |
| | 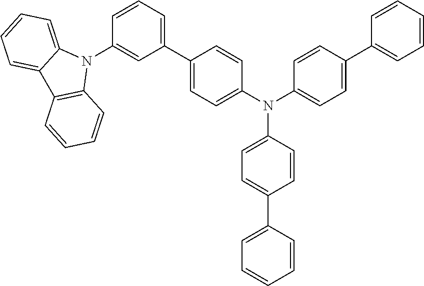 [SMILES: c1ccc(-c2ccc(N(c3ccc(-c4ccccc4)cc3)c3ccc(-c4cccc(-n5c6ccccc6c6ccccc65)c4)cc3)cc2)cc1] |
| | HT-4 |
| Light-Emitting Layer | 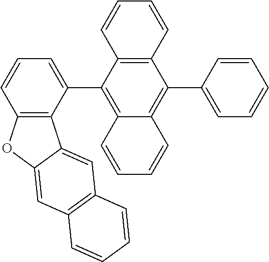 [SMILES: c1ccc(-c2c3ccccc3c(-c3cccc4oc5cc6ccccc6cc5c34)c3ccccc23)cc1] |
| | H-1 |
| | 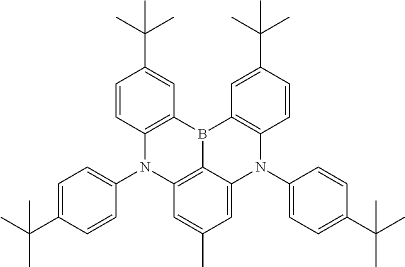 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1] |
| | D-1 |
| Electron Transport Layer/ Hole-Blocking layer/Electron Injection Layer | 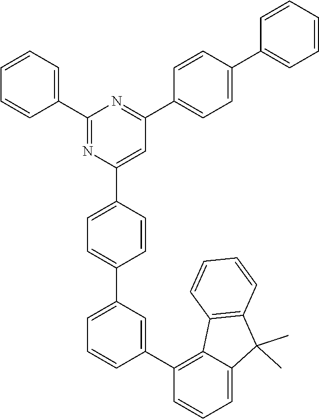 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 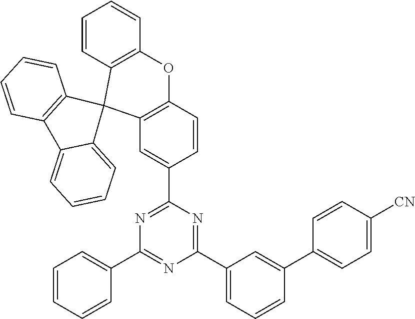 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ET-2 |
| | 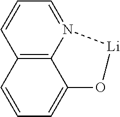 |
| | EI-1 |
| N-Type Charge Generation Layer | 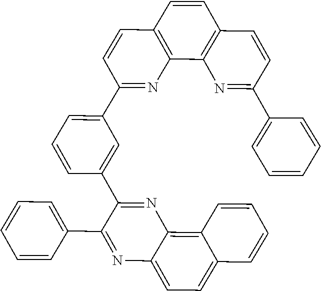 [SMILES: c1ccc(-c2ccc3ccc4ccc(-c5cccc(-c6nc7c(ccc8ccccc87)nc6-c6ccccc6)c5)nc4c3n2)cc1] |
| | C-5 |
| | 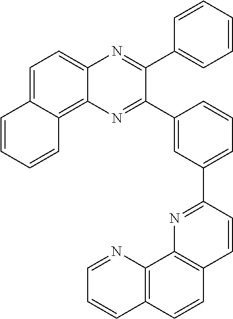 [SMILES: c1ccc(-c2nc3ccc4ccccc4c3nc2-c2cccc(-c3ccc4ccc5cccnc5c4n3)c2)cc1] |
| | C-2 |
| | 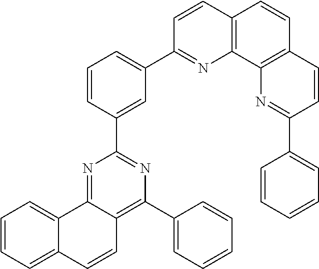 [SMILES: c1ccc(-c2ccc3ccc4ccc(-c5cccc(-c6nc(-c7ccccc7)c7ccc8ccccc8c7n6)c5)nc4c3n2)cc1] |
| | C-9 |
| | 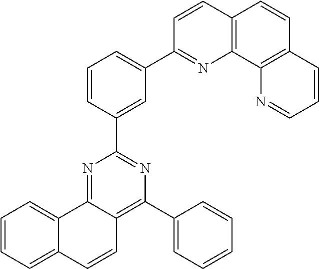 [SMILES: c1ccc(-c2nc(-c3cccc(-c4ccc5ccc6cccnc6c5n4)c3)nc3c2ccc2ccccc23)cc1] |
| | C-10 |
| | 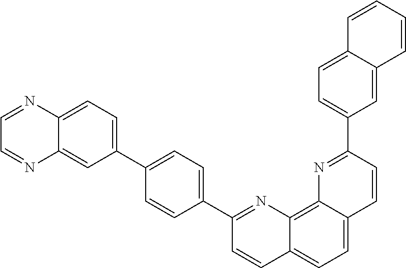 [SMILES: c1ccc2cc(-c3ccc4ccc5ccc(-c6ccc(-c7ccc8nccnc8c7)cc6)nc5c4n3)ccc2c1] |
| | T-1 |
| | 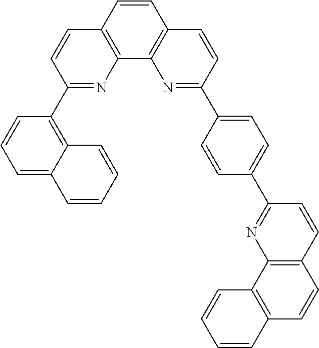 [SMILES: c1ccc2c(-c3ccc4ccc5ccc(-c6ccc(-c7ccc8ccc9ccccc9c8n7)cc6)nc5c4n3)cccc2c1] |
| | T-2 |

1. An organic electroluminescent compound represented by the following Formula 1: 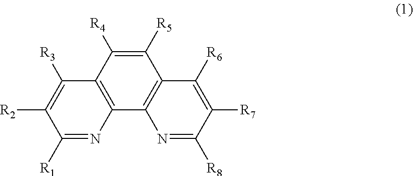
wherein in Formula 1,
R₁ represents hydrogen, deuterium, a halogen, a cyano, a substituted or unsubstituted phenyl, a substituted or unsubstituted biphenyl, a substituted or unsubstituted terphenyl, a substituted or unsubstituted (C₁-C₃₀)alkyl, or a substituted or unsubstituted (3- to 30-membered)heteroaryl; and
R₂ to R₈ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or HAr defined by the following Formula 1-a; or may be linked to an adjacent substituent(s) to form a ring(s);
with a proviso that at least one of R₂ to R₈ is represented by HAr defined by Formula 1-a, 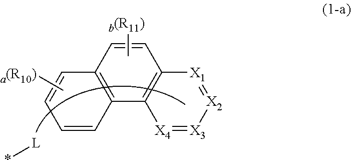
wherein X₁ to X₄ each independently represent N or CR₉;
L represents a single bond, a substituted or unsubstituted (C₆-C₃₀)arylene, or a substituted or unsubstituted (3- to 30-membered)heteroarylene;
R₉ to R₁₁ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or are bonded to L; or may be linked to an adjacent substituent(s) to form a ring(s); and
a represents an integer of 1 to 4, b represents an integer of 1 or 2, and if a and b are integers of 2 or more, each a and each b may be the same as or different from each other,
with a proviso that if R₈ is HAr in Formula 1, and at least one of X₁ to X₄ of Formula 1-a is N, R₁ is hydrogen, deuterium, a halogen, a cyano, a substituted or unsubstituted (C₁-C₃₀)alkyl, a substituted or unsubstituted phenyl, a substituted or unsubstituted biphenyl, or a substituted or unsubstituted terphenyl.

2. The organic electroluminescent compound according to claim 1, wherein the substituted phenyl, the substituted biphenyl, the substituted terphenyl, the substituted alkyl, the substituted alkenyl, the substituted aryl, the substituted arylene, the substituted heteroaryl, the substituted heteroarylene, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, and the fused ring group of the substituted aliphatic ring(s) and aromatic ring(s) each independently are substituted with at least one selected from the group consisting of deuterium, a halogen, a cyano, a carboxyl, a nitro, a hydroxy, a (C₁-C₃₀)alkyl, a halo(C₁-C₃₀)alkyl, a (C₂-C₃₀)alkenyl, a (C₂-C₃₀)alkynyl, a (C₁-C₃₀)alkoxy, a (C₁-C₃₀)alkylthio, a (C₃-C₃₀)cycloalkyl, a (C₃-C₃₀)cycloalkenyl, a (3-to 7-membered)heterocycloalkyl, a (C₆-C₃₀)aryloxy, a (C₆-C₃₀)arylthio, a (5- to 30-membered)heteroaryl unsubstituted or substituted with a (C₆-C₃₀)aryl, a (C₆-C₃₀)aryl unsubstituted or substituted with a (5- to 30-membered)heteroaryl, a tri(C₁-C₃₀)alkylsilyl, a tri(C₆-C₃₀)arylsilyl, a di(C₁-C₃₀)alkyl(C₆-C₃₀)arylsilyl, a (C₁-C₃₀)alkyldi(C₆-C₃₀)arylsilyl, a fused ring group of a (C₃-C₃₀) aliphatic ring(s) and a (C₆-C₃₀) aromatic ring(s), an amino, a mono- or di(C₁-C₃₀)alkylamino, a mono- or di(C₆-C₃₀)arylamino, a (C₁-C₃₀)alkyl(C₆-C₃₀)arylamino, a mono- or di(3- to 30-membered)heteroarylamino, a (C₁-C₃₀)alkyl(3- to 30-membered)heteroarylamino, a (C₆-C₃₀)aryl(3- to 30-membered)heteroarylamino, a (C₁-C₃₀)alkylcarbonyl, a (C₁-C₃₀)alkoxycarbonyl, a (C₆-C₃₀)arylcarbonyl, a (C₆-C₃₀)arylphosphinyl, a di(C₆-C₃₀)arylboronyl, a di(C₁-C₃₀)alkylboronyl, a (C₁-C₃₀)alkyl(C₆-C₃₀)arylboronyl, a (C₆-C₃₀)aryl(C₁-C₃₀)alkyl, a (C₁-C₃₀)alkyl(C₆-C₃₀)aryl, and combinations thereof.

3. The organic electroluminescent compound according to claim 1, wherein Formula 1 is represented by any one of the following Formulas 1-1 to 1-7: 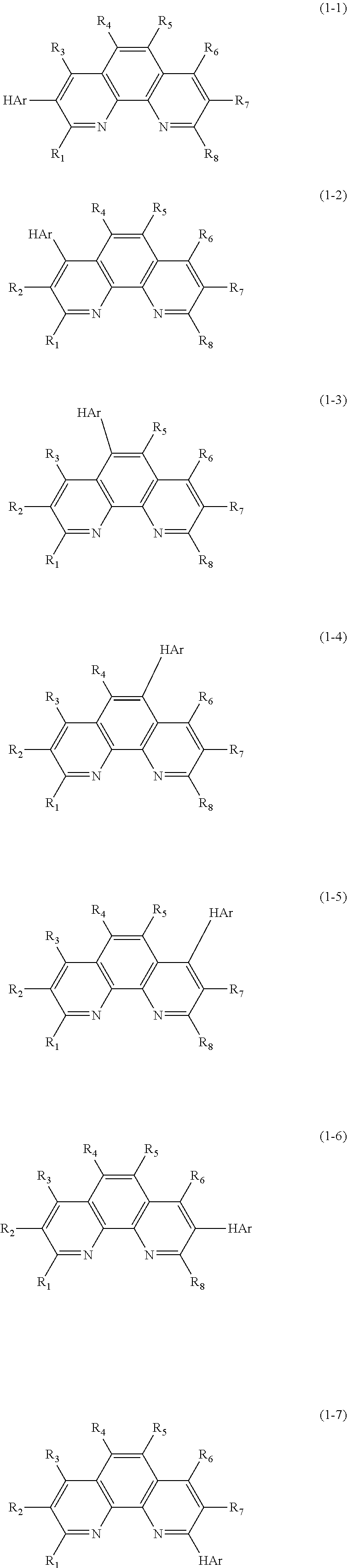
wherein in Formulas 1-1 to 1-7,
R₁ and HAr are as defined in claim 1; and
R₂ to R₈ each independently represent hydrogen, deuterium, a halogen, a cyano, a substituted or unsubstituted (C₁-C₃₀)alkyl, a substituted or unsubstituted (C₆-C₃₀)aryl, or a substituted or unsubstituted (3- to 30-membered)heteroaryl.

4. The organic electroluminescent compound according to claim 1, wherein Formula 1-a is represented by any one of the following Formulas 1-a-1 to 1-a-10: 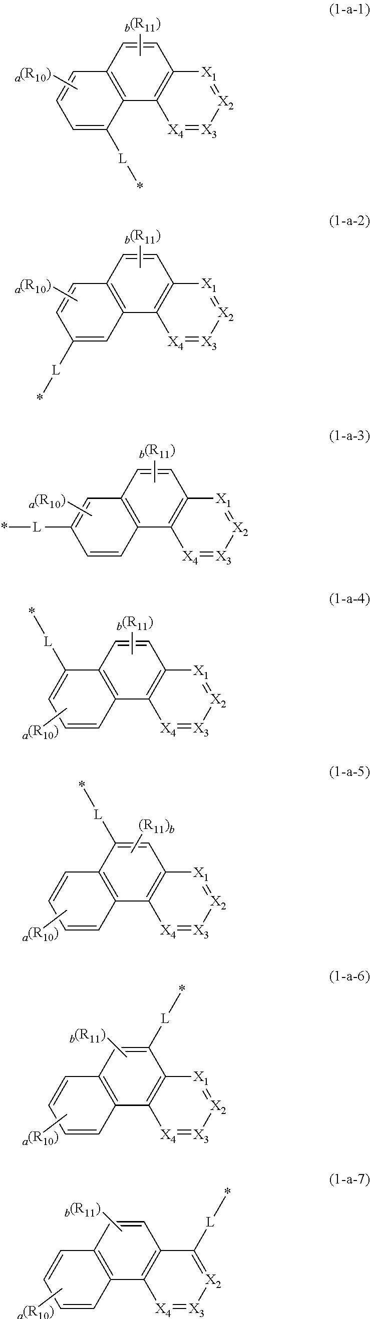 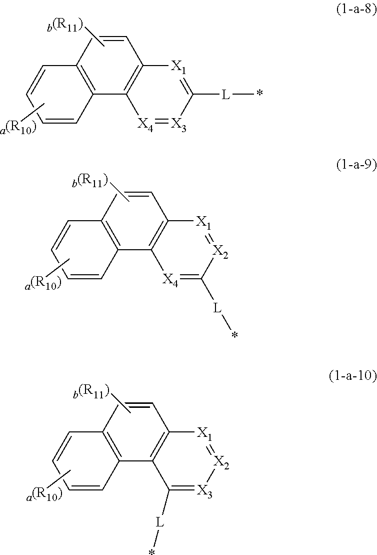
wherein in Formulas 1-a-1 to 1-a-10,
R₁₀, R₁₁, L, X₁ to X₄, a, and b are as defined in claim 1.

5. The organic electroluminescent compound according to claim 1, wherein the compound represented by Formula 1 is selected from the following compounds: 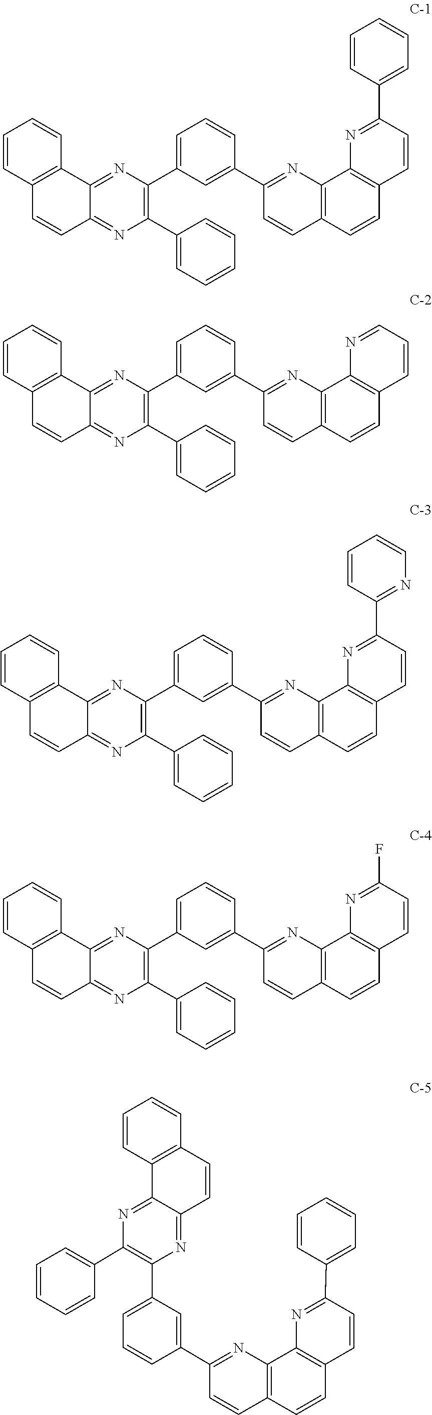 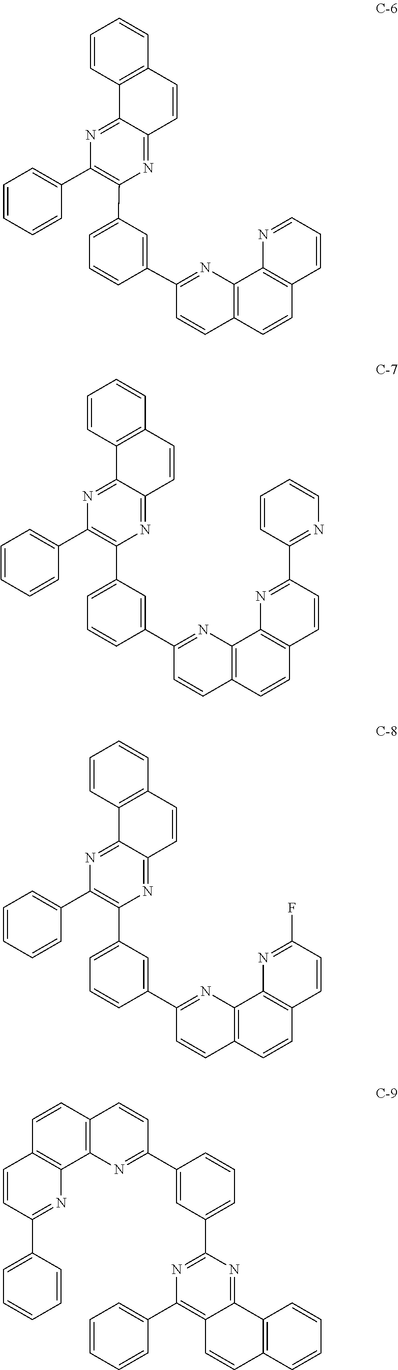 [SMILES: c1ccc(-c2nc3c(ccc4ccccc43)nc2-c2cccc(-c3ccc4ccc5cccnc5c4n3)c2)cc1 ; c1ccc(-c2nc3c(ccc4ccccc43)nc2-c2cccc(-c3ccc4ccc5ccc(-c6ccccn6)nc5c4n3)c2)cc1 ; Fc1ccc2ccc3ccc(-c4cccc(-c5nc6ccc7ccccc7c6nc5-c5ccccc5)c4)nc3c2n1 ; c1ccc(-c2ccc3ccc4ccc(-c5cccc(-c6nc(-c7ccccc7)c7ccc8ccccc8c7n6)c5)nc4c3n2)cc1] 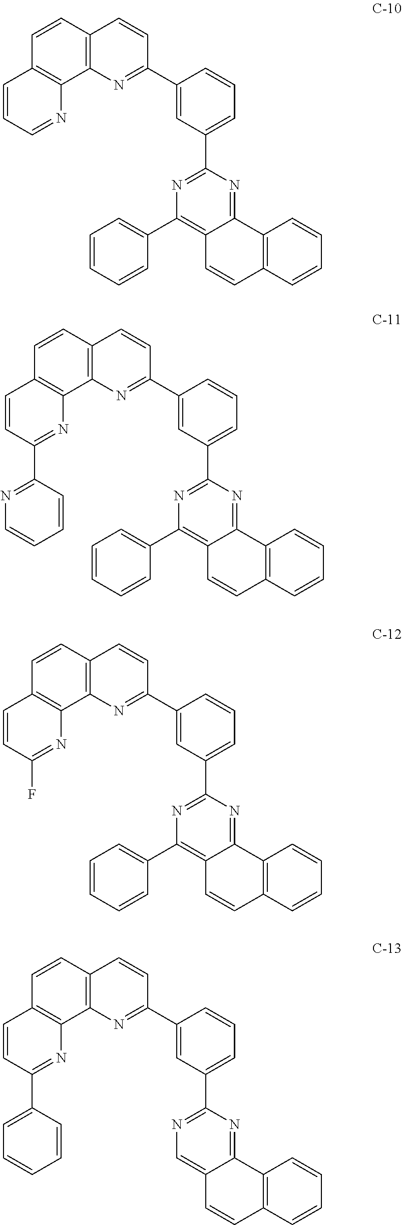 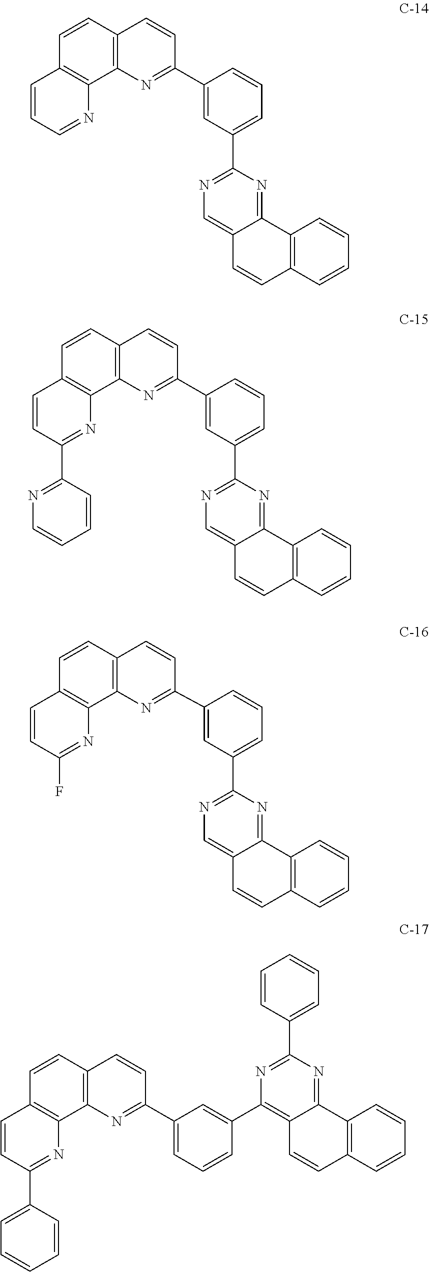 [SMILES: c1cc(-c2ccc3ccc4cccnc4c3n2)cc(-c2ncc3ccc4ccccc4c3n2)c1 ; c1ccc(-c2ccc3ccc4ccc(-c5cccc(-c6ncc7ccc8ccccc8c7n6)c5)nc4c3n2)nc1 ; Fc1ccc2ccc3ccc(-c4cccc(-c5ncc6ccc7ccccc7c6n5)c4)nc3c2n1 ; c1ccc(-c2ccc3ccc4ccc(-c5cccc(-c6nc(-c7ccccc7)nc7c6ccc6ccccc67)c5)nc4c3n2)cc1] 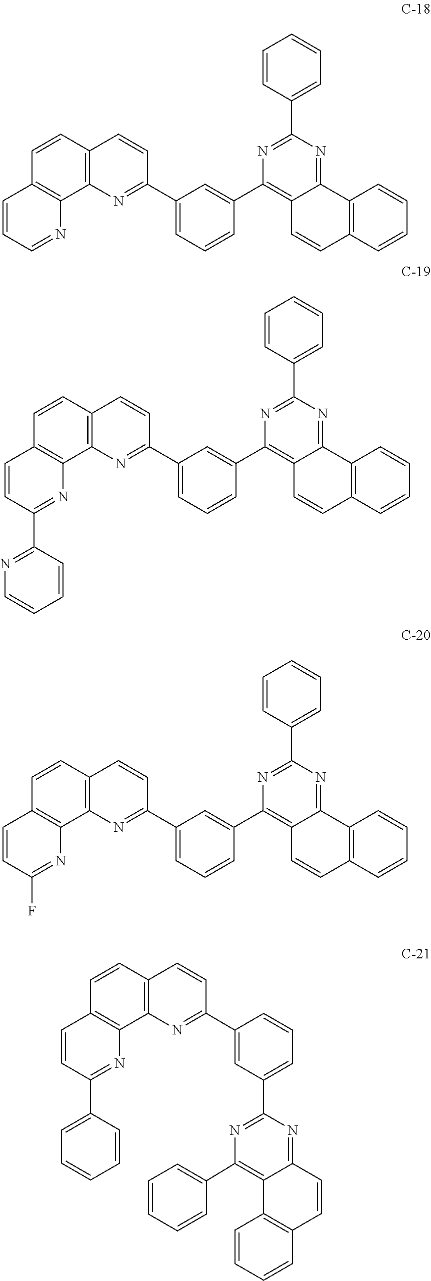 [SMILES: c1ccc(-c2nc(-c3cccc(-c4ccc5ccc6cccnc6c5n4)c3)c3ccc4ccccc4c3n2)cc1 ; c1ccc(-c2nc(-c3cccc(-c4ccc5ccc6ccc(-c7ccccn7)nc6c5n4)c3)c3ccc4ccccc4c3n2)cc1 ; Fc1ccc2ccc3ccc(-c4cccc(-c5nc(-c6ccccc6)nc6c5ccc5ccccc56)c4)nc3c2n1 ; c1ccc(-c2ccc3ccc4ccc(-c5cccc(-c6nc(-c7ccccc7)c7c(ccc8ccccc87)n6)c5)nc4c3n2)cc1] 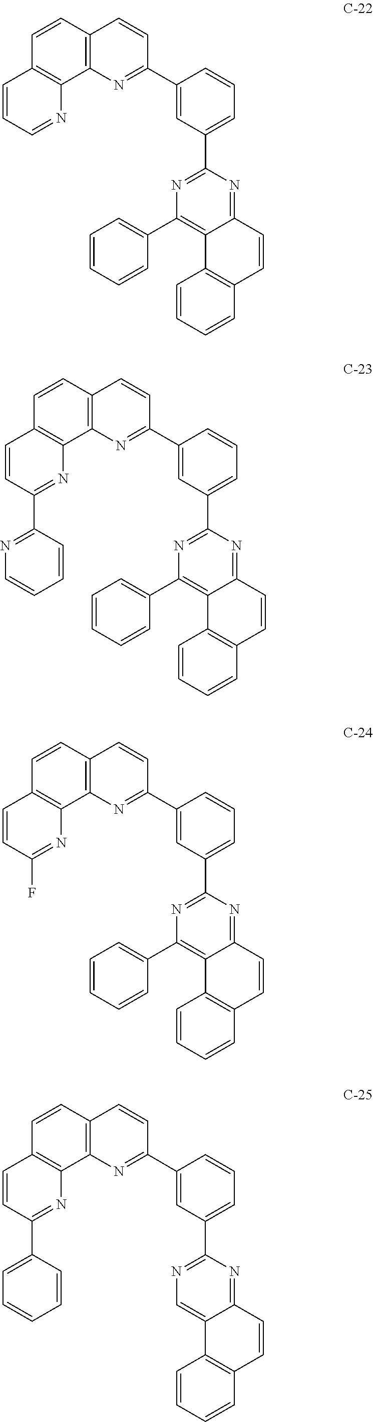 [SMILES: c1ccc(-c2nc(-c3cccc(-c4ccc5ccc6cccnc6c5n4)c3)nc3ccc4ccccc4c23)cc1 ; c1ccc(-c2nc(-c3cccc(-c4ccc5ccc6ccc(-c7ccccn7)nc6c5n4)c3)nc3ccc4ccccc4c23)cc1 ; Fc1ccc2ccc3ccc(-c4cccc(-c5nc(-c6ccccc6)c6c(ccc7ccccc76)n5)c4)nc3c2n1 ; c1ccc(-c2ccc3ccc4ccc(-c5cccc(-c6ncc7c(ccc8ccccc87)n6)c5)nc4c3n2)cc1] 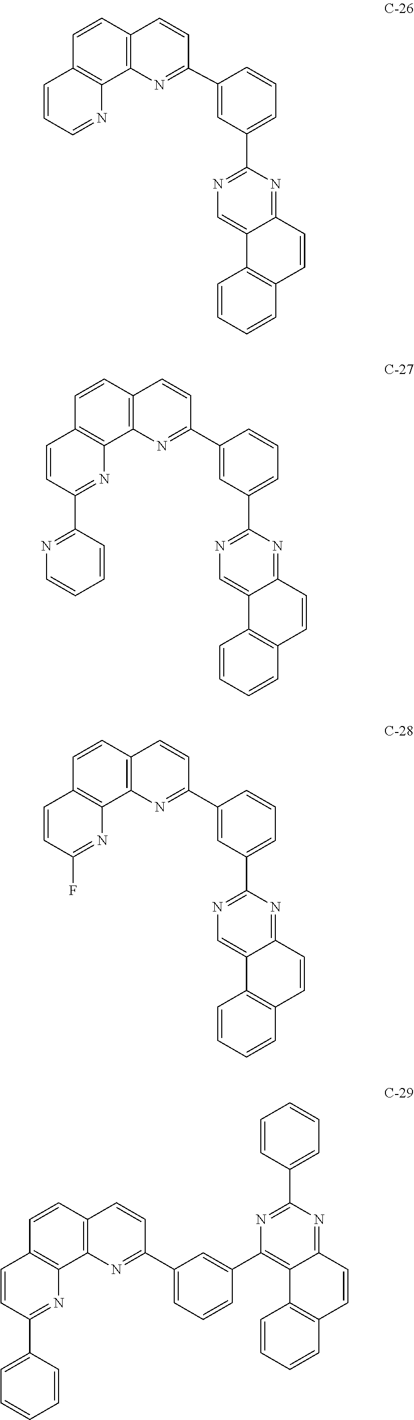 [SMILES: c1cc(-c2ccc3ccc4cccnc4c3n2)cc(-c2ncc3c(ccc4ccccc43)n2)c1 ; c1ccc(-c2ccc3ccc4ccc(-c5cccc(-c6ncc7c(ccc8ccccc87)n6)c5)nc4c3n2)nc1 ; Fc1ccc2ccc3ccc(-c4cccc(-c5ncc6c(ccc7ccccc76)n5)c4)nc3c2n1 ; c1ccc(-c2ccc3ccc4ccc(-c5cccc(-c6nc(-c7ccccc7)nc7ccc8ccccc8c67)c5)nc4c3n2)cc1] 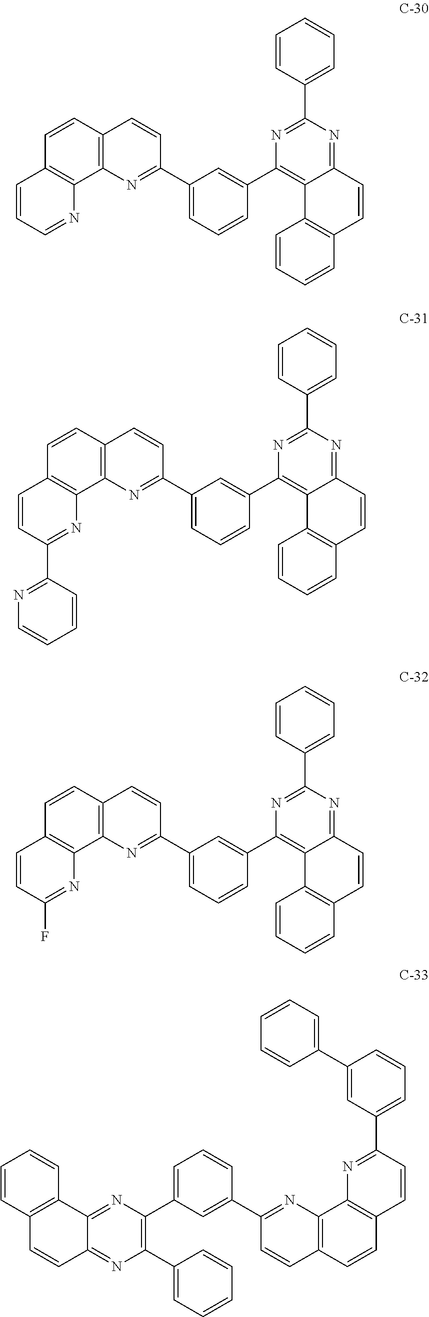 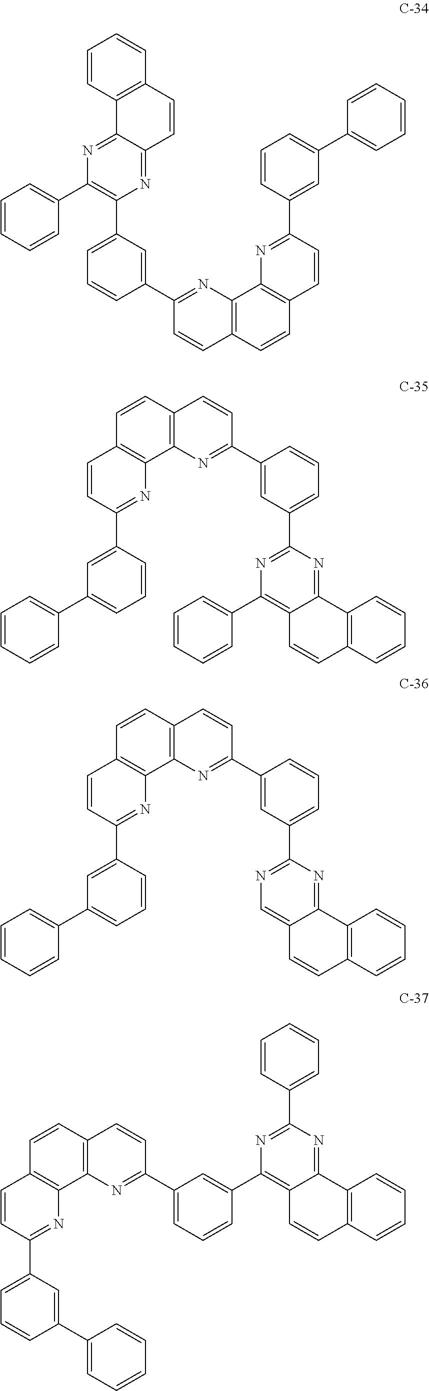 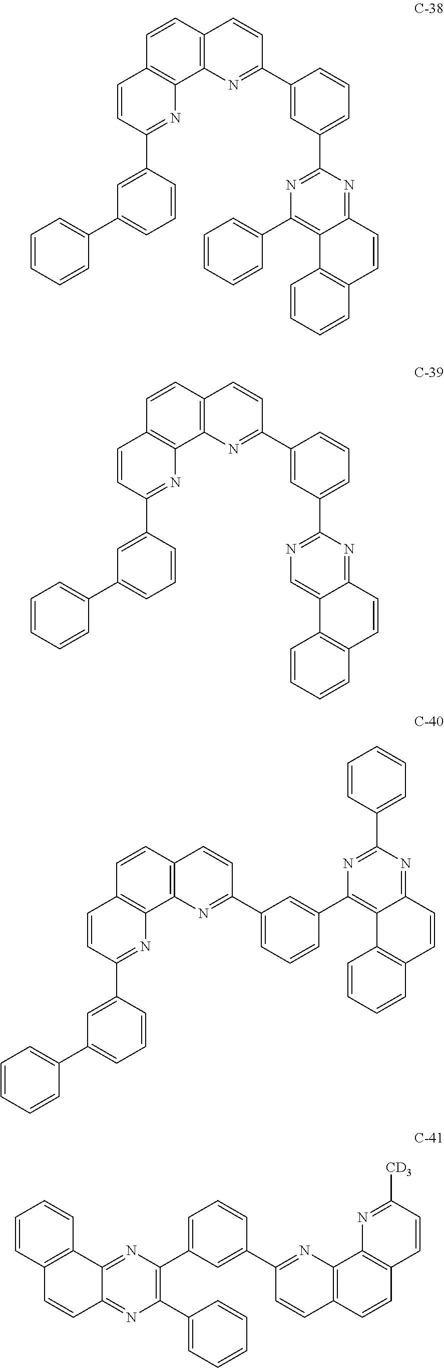 [SMILES: c1ccc(-c2cccc(-c3ccc4ccc5ccc(-c6cccc(-c7nc(-c8ccccc8)c8c(ccc9ccccc98)n7)c6)nc5c4n3)c2)cc1 ; c1ccc(-c2cccc(-c3ccc4ccc5ccc(-c6cccc(-c7ncc8c(ccc9ccccc98)n7)c6)nc5c4n3)c2)cc1 ; c1ccc(-c2cccc(-c3ccc4ccc5ccc(-c6cccc(-c7nc(-c8ccccc8)nc8ccc9ccccc9c78)c6)nc5c4n3)c2)cc1 ; [2H]C([2H])([2H])c1ccc2ccc3ccc(-c4cccc(-c5nc6c(ccc7ccccc76)nc5-c5ccccc5)c4)nc3c2n1] 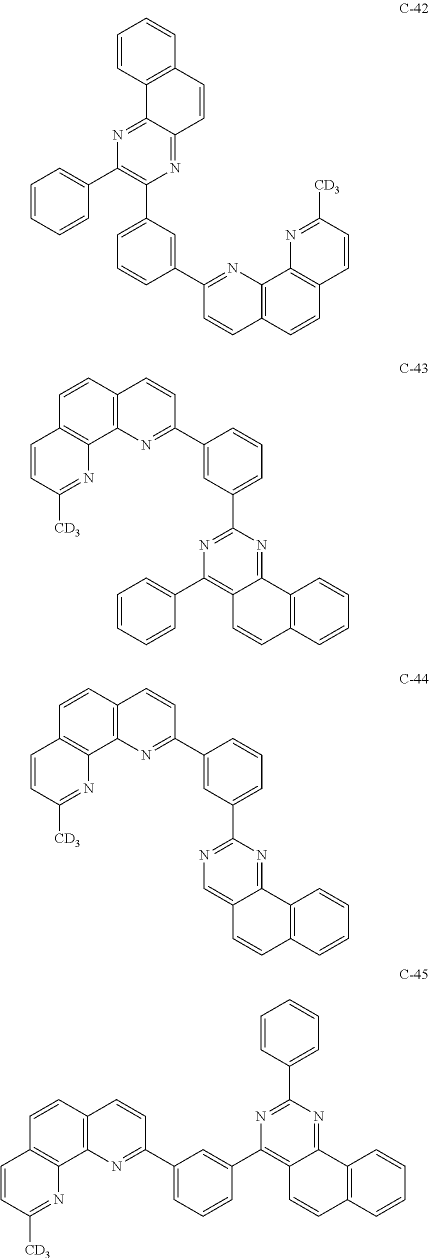 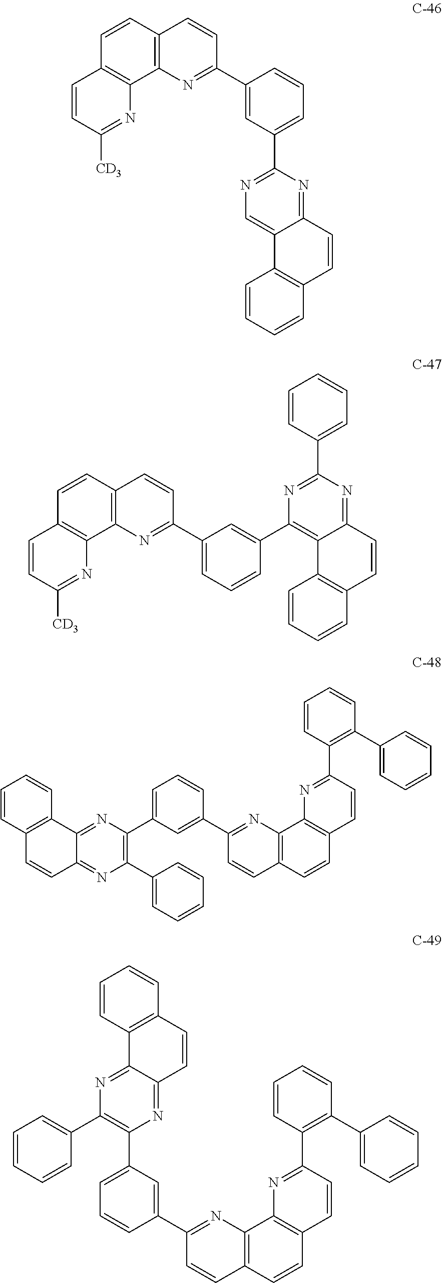 [SMILES: [2H]C([2H])([2H])c1ccc2ccc3ccc(-c4cccc(-c5ncc6c(ccc7ccccc76)n5)c4)nc3c2n1 ; [2H]C([2H])([2H])c1ccc2ccc3ccc(-c4cccc(-c5nc(-c6ccccc6)nc6ccc7ccccc7c56)c4)nc3c2n1 ; c1ccc(-c2ccccc2-c2ccc3ccc4ccc(-c5cccc(-c6nc7c(ccc8ccccc87)nc6-c6ccccc6)c5)nc4c3n2)cc1 ; c1ccc(-c2ccccc2-c2ccc3ccc4ccc(-c5cccc(-c6nc7ccc8ccccc8c7nc6-c6ccccc6)c5)nc4c3n2)cc1] 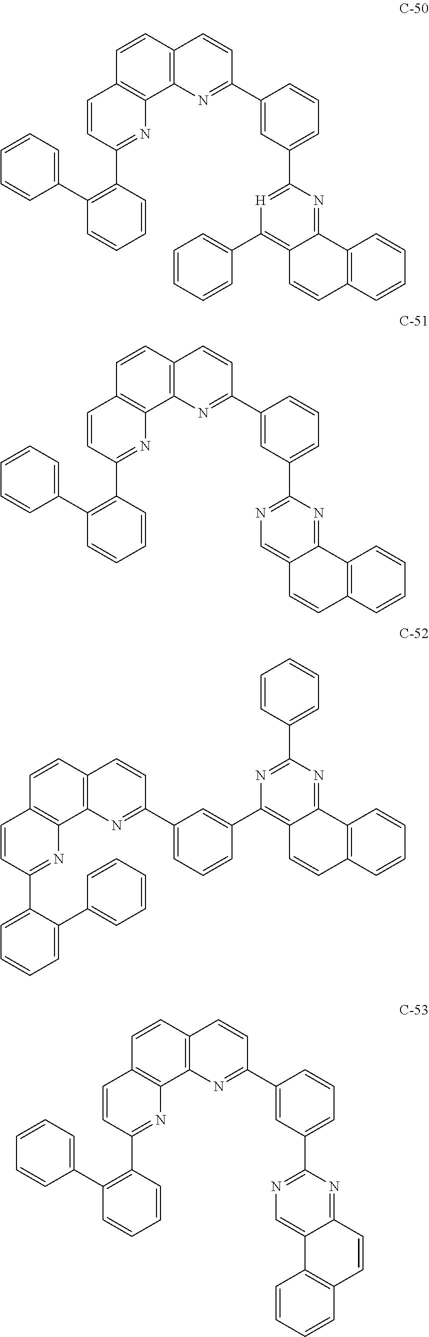 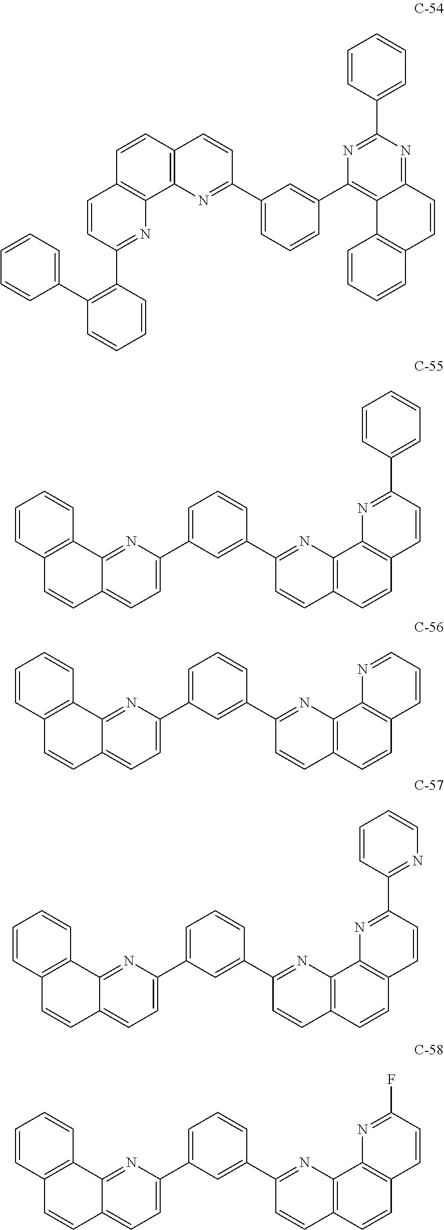 [SMILES: c1ccc(-c2nc(-c3cccc(-c4ccc5ccc6ccc(-c7ccccc7-c7ccccc7)nc6c5n4)c3)c3c(ccc4ccccc43)n2)cc1 ; c1ccc(-c2ccc3ccc4ccc(-c5cccc(-c6ccc7ccc8ccccc8c7n6)c5)nc4c3n2)cc1 ; c1cc(-c2ccc3ccc4ccccc4c3n2)cc(-c2ccc3ccc4cccnc4c3n2)c1 ; c1ccc(-c2ccc3ccc4ccc(-c5cccc(-c6ccc7ccc8ccccc8c7n6)c5)nc4c3n2)nc1 ; Fc1ccc2ccc3ccc(-c4cccc(-c5ccc6ccc7ccccc7c6n5)c4)nc3c2n1] 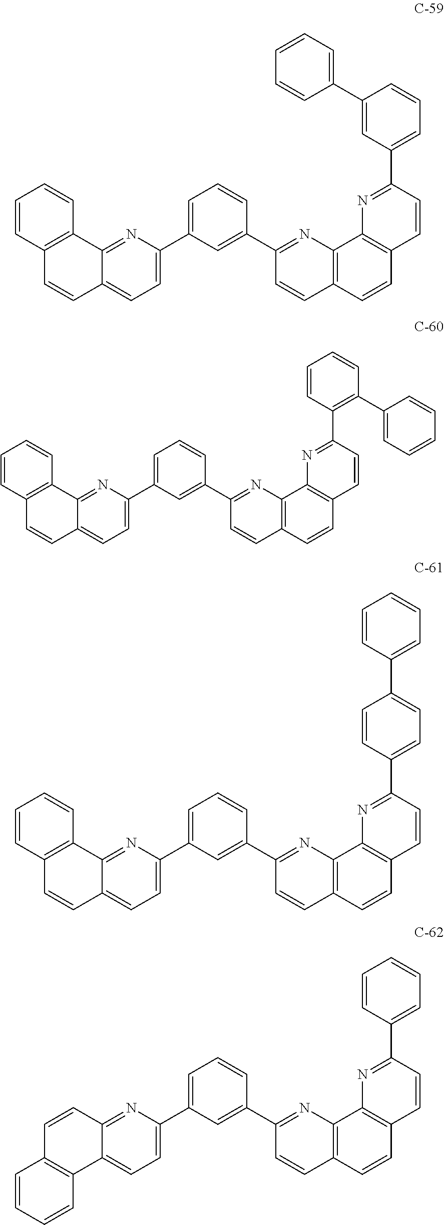 [SMILES: c1ccc(-c2cccc(-c3ccc4ccc5ccc(-c6cccc(-c7ccc8ccc9ccccc9c8n7)c6)nc5c4n3)c2)cc1 ; c1ccc(-c2ccccc2-c2ccc3ccc4ccc(-c5cccc(-c6ccc7ccc8ccccc8c7n6)c5)nc4c3n2)cc1 ; c1ccc(-c2ccc(-c3ccc4ccc5ccc(-c6cccc(-c7ccc8ccc9ccccc9c8n7)c6)nc5c4n3)cc2)cc1 ; c1ccc(-c2ccc3ccc4ccc(-c5cccc(-c6ccc7c(ccc8ccccc87)n6)c5)nc4c3n2)cc1] 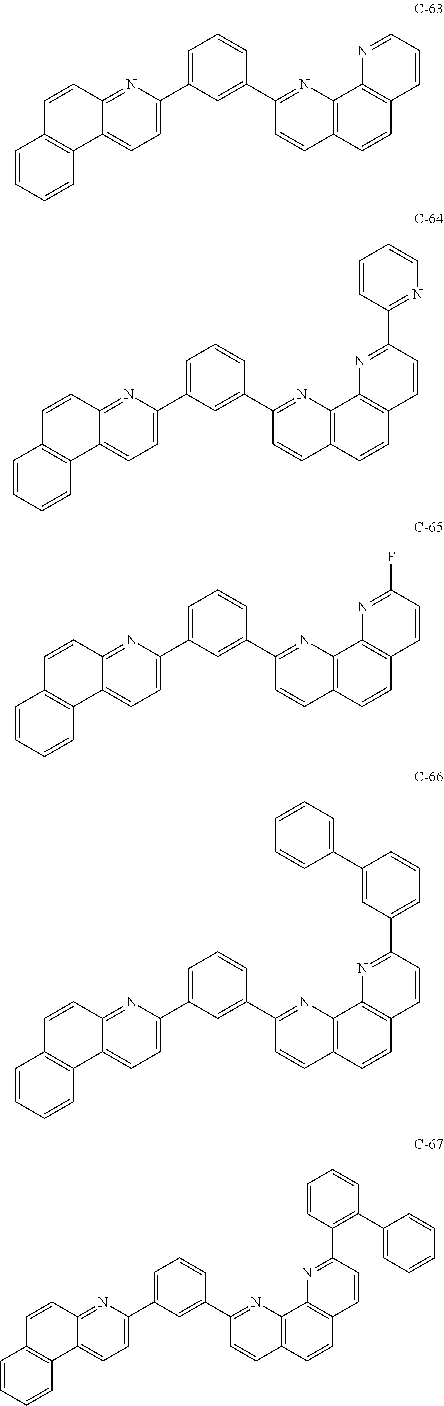 [SMILES: c1cc(-c2ccc3c(ccc4ccccc43)n2)cc(-c2ccc3ccc4cccnc4c3n2)c1 ; c1ccc(-c2ccc3ccc4ccc(-c5cccc(-c6ccc7c(ccc8ccccc87)n6)c5)nc4c3n2)nc1 ; Fc1ccc2ccc3ccc(-c4cccc(-c5ccc6c(ccc7ccccc76)n5)c4)nc3c2n1 ; c1ccc(-c2cccc(-c3ccc4ccc5ccc(-c6cccc(-c7ccc8c(ccc9ccccc98)n7)c6)nc5c4n3)c2)cc1 ; c1ccc(-c2ccccc2-c2ccc3ccc4ccc(-c5cccc(-c6ccc7c(ccc8ccccc87)n6)c5)nc4c3n2)cc1] 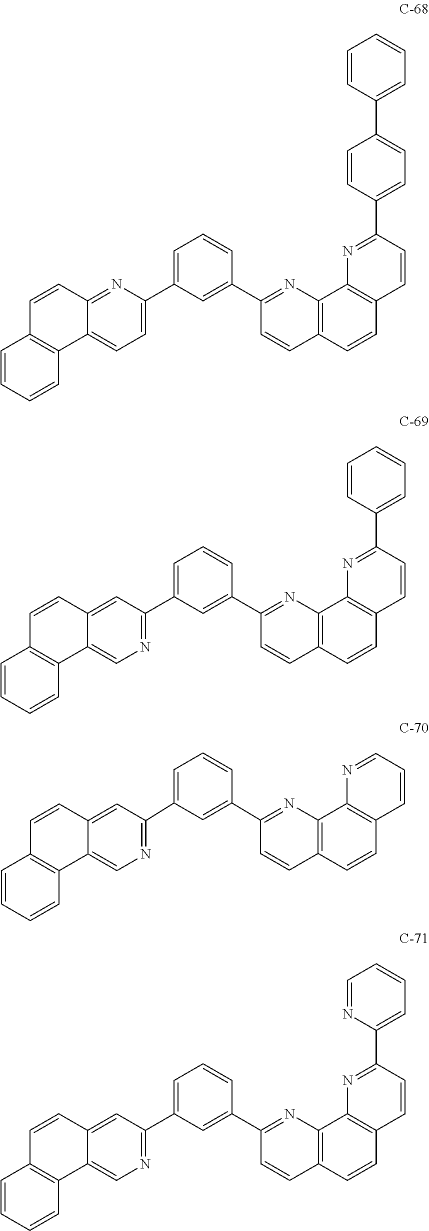 [SMILES: c1ccc(-c2ccc(-c3ccc4ccc5ccc(-c6cccc(-c7ccc8c(ccc9ccccc98)n7)c6)nc5c4n3)cc2)cc1 ; c1ccc(-c2ccc3ccc4ccc(-c5cccc(-c6cc7ccc8ccccc8c7cn6)c5)nc4c3n2)cc1 ; c1cc(-c2cc3ccc4ccccc4c3cn2)cc(-c2ccc3ccc4cccnc4c3n2)c1 ; c1ccc(-c2ccc3ccc4ccc(-c5cccc(-c6cc7ccc8ccccc8c7cn6)c5)nc4c3n2)nc1] 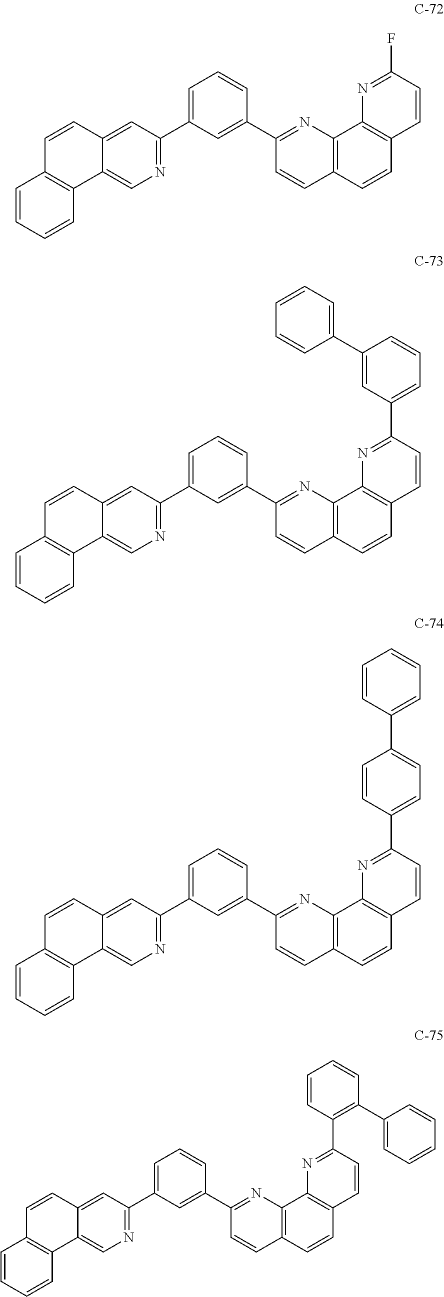 [SMILES: Fc1ccc2ccc3ccc(-c4cccc(-c5cc6ccc7ccccc7c6cn5)c4)nc3c2n1 ; c1ccc(-c2cccc(-c3ccc4ccc5ccc(-c6cccc(-c7cc8ccc9ccccc9c8cn7)c6)nc5c4n3)c2)cc1 ; c1ccc(-c2ccc(-c3ccc4ccc5ccc(-c6cccc(-c7cc8ccc9ccccc9c8cn7)c6)nc5c4n3)cc2)cc1 ; c1ccc(-c2ccccc2-c2ccc3ccc4ccc(-c5cccc(-c6cc7ccc8ccccc8c7cn6)c5)nc4c3n2)cc1] 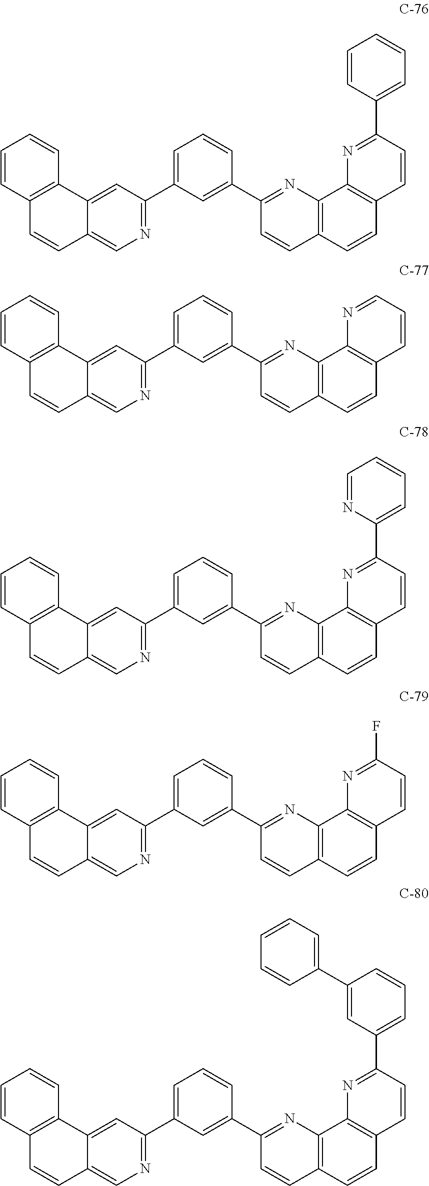 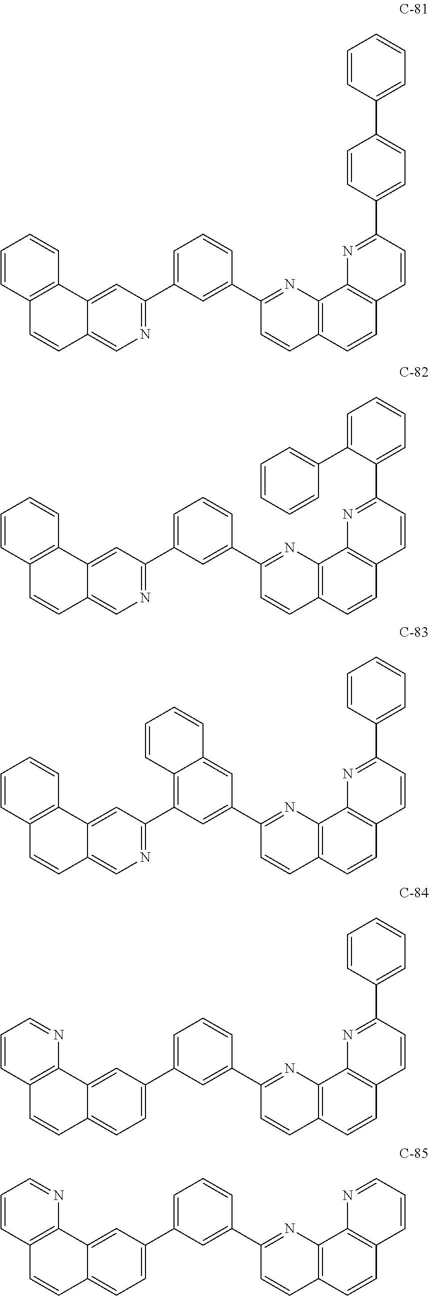 [SMILES: c1ccc(-c2ccc(-c3ccc4ccc5ccc(-c6cccc(-c7cc8c(ccc9ccccc98)cn7)c6)nc5c4n3)cc2)cc1 ; c1ccc(-c2ccccc2-c2ccc3ccc4ccc(-c5cccc(-c6cc7c(ccc8ccccc87)cn6)c5)nc4c3n2)cc1 ; c1ccc(-c2ccc3ccc4ccc(-c5cc(-c6cc7c(ccc8ccccc87)cn6)c6ccccc6c5)nc4c3n2)cc1 ; c1ccc(-c2ccc3ccc4ccc(-c5cccc(-c6ccc7ccc8cccnc8c7c6)c5)nc4c3n2)cc1 ; c1cc(-c2ccc3ccc4cccnc4c3c2)cc(-c2ccc3ccc4cccnc4c3n2)c1] 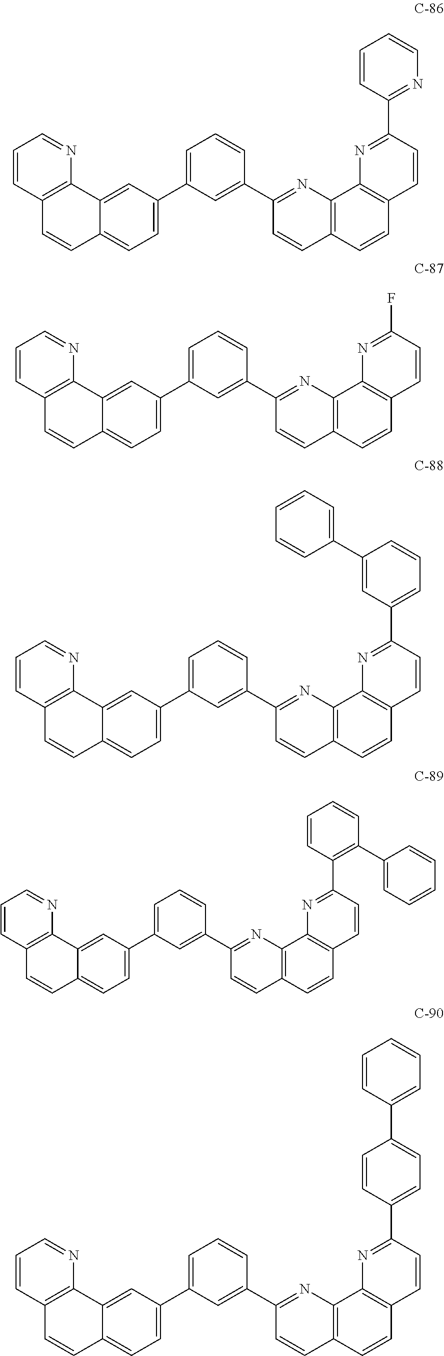 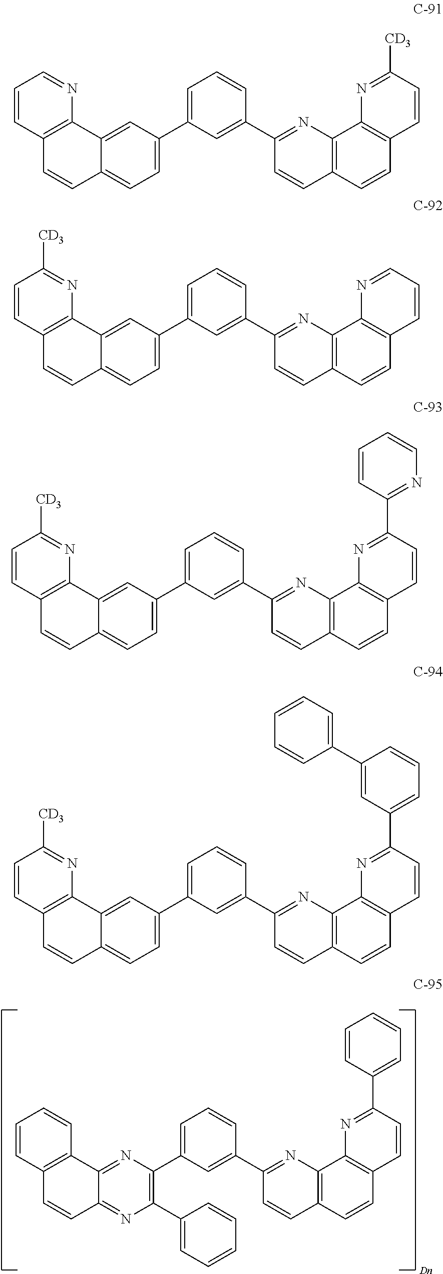 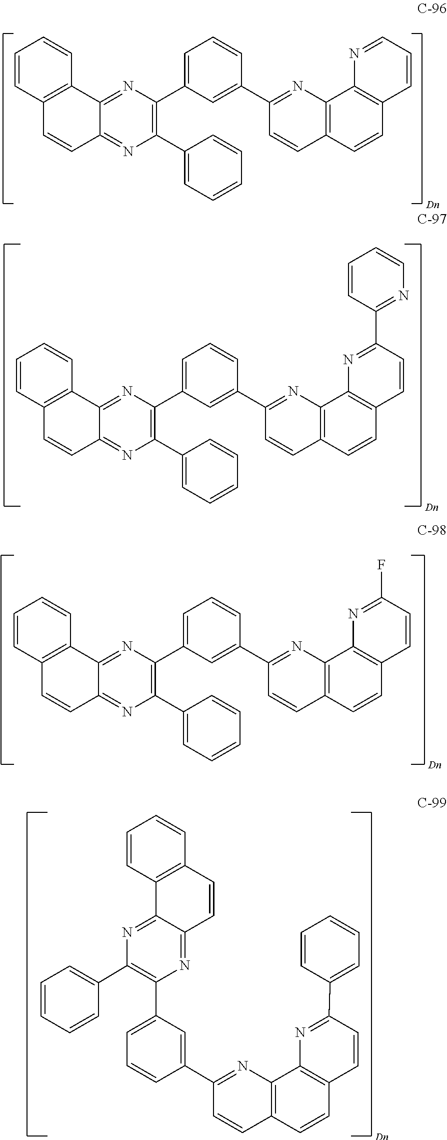 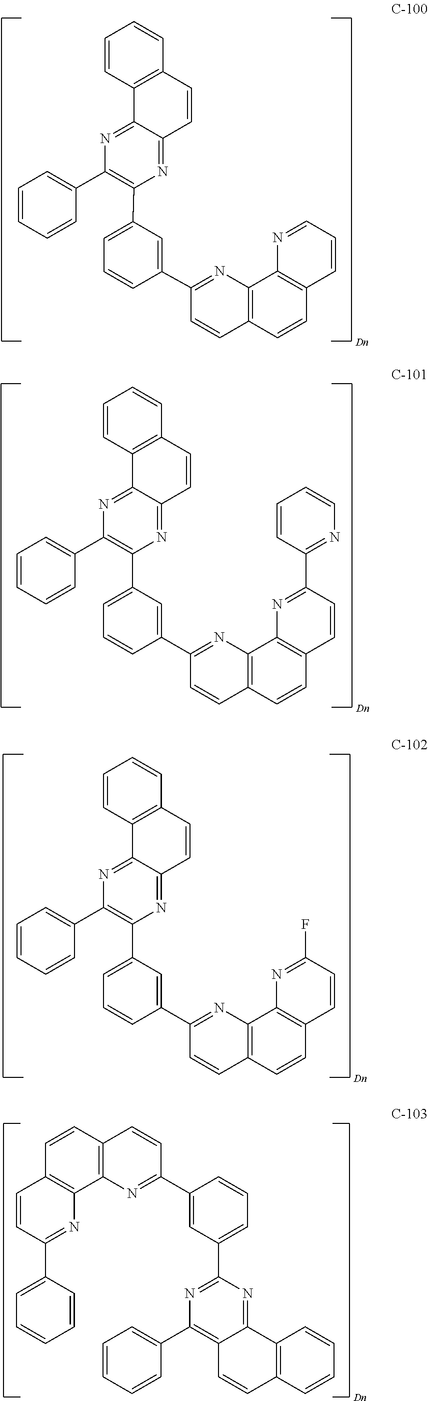 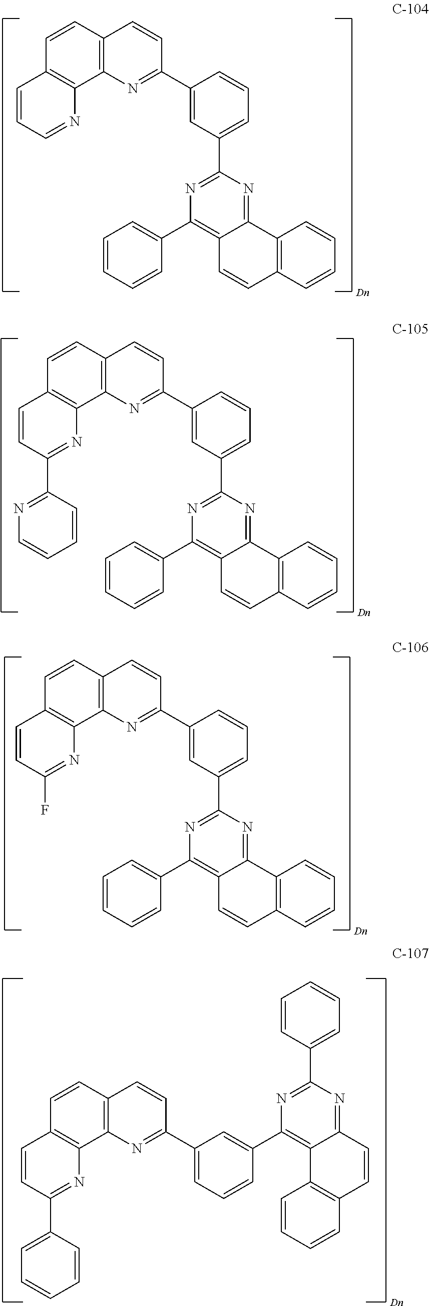 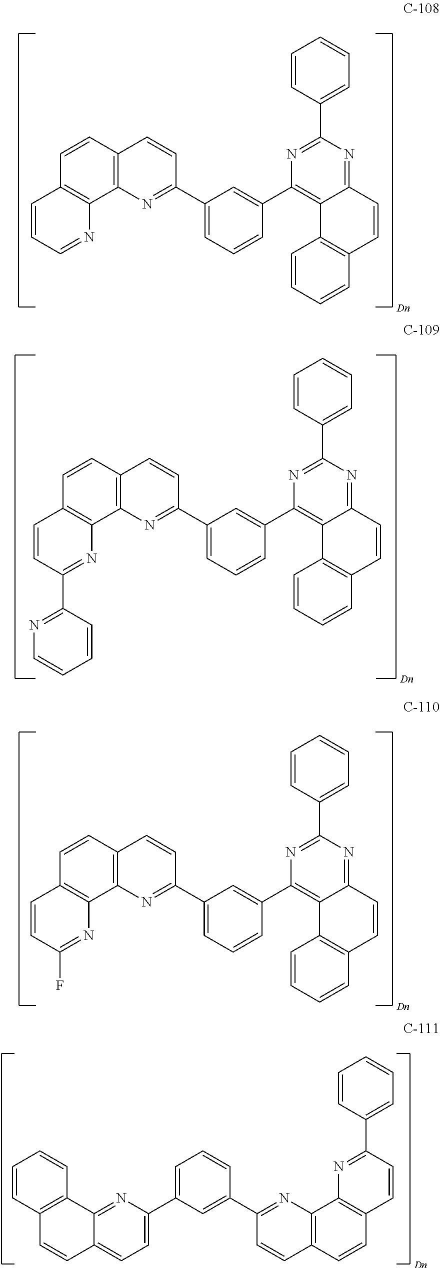 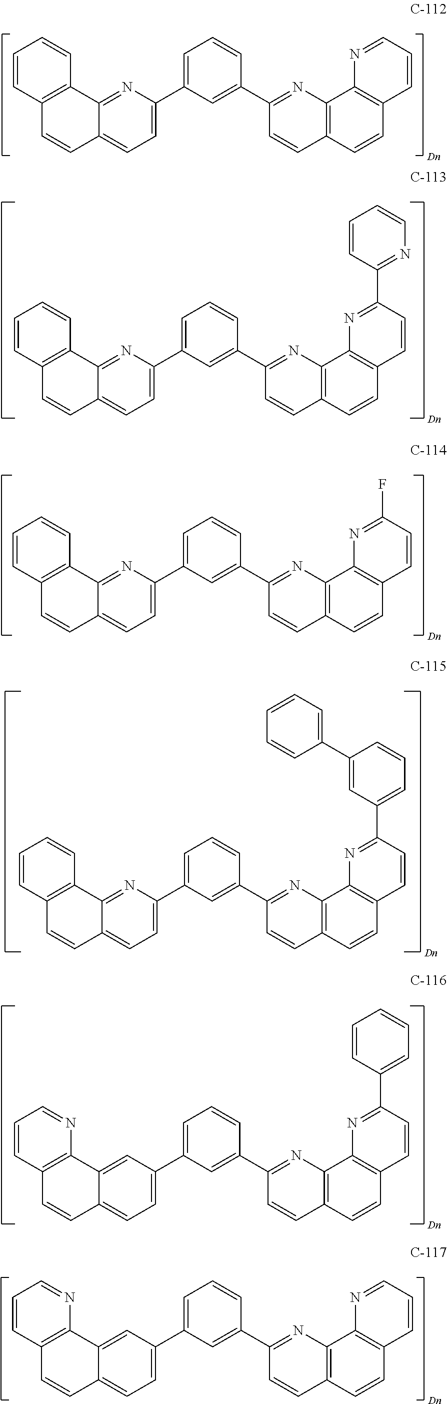 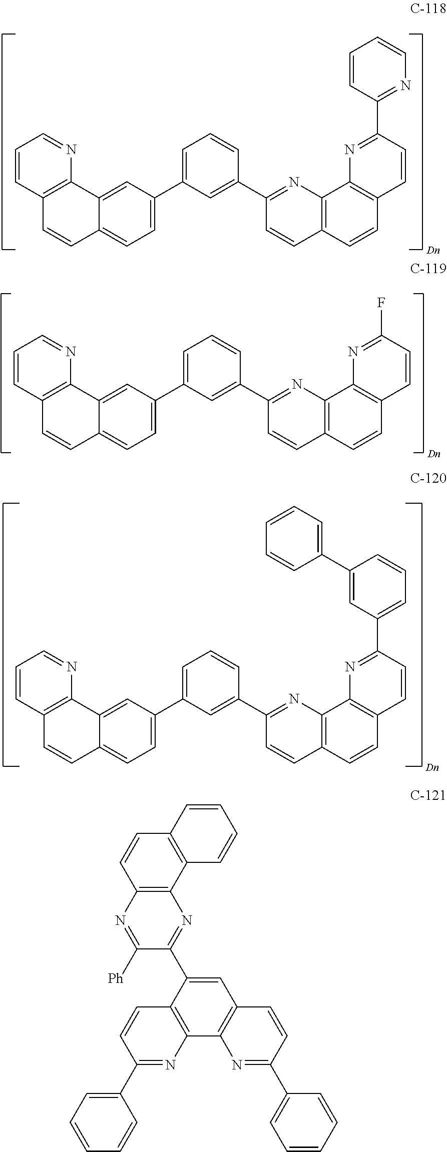 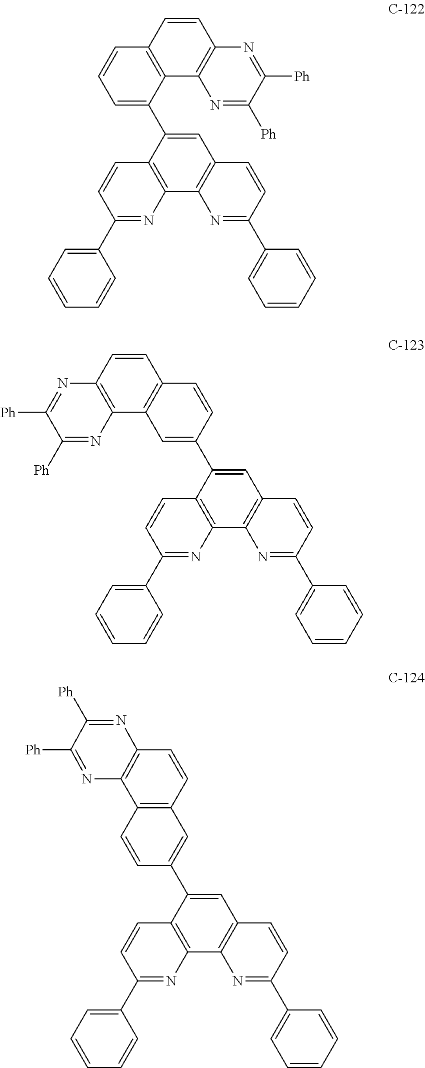 [SMILES: c1ccc(-c2ccc3cc(-c4cccc5ccc6nc(-c7ccccc7)c(-c7ccccc7)nc6c45)c4ccc(-c5ccccc5)nc4c3n2)cc1 ; c1ccc(-c2ccc3cc(-c4ccc5ccc6nc(-c7ccccc7)c(-c7ccccc7)nc6c5c4)c4ccc(-c5ccccc5)nc4c3n2)cc1 ; c1ccc(-c2ccc3cc(-c4ccc5c(ccc6nc(-c7ccccc7)c(-c7ccccc7)nc65)c4)c4ccc(-c5ccccc5)nc4c3n2)cc1] 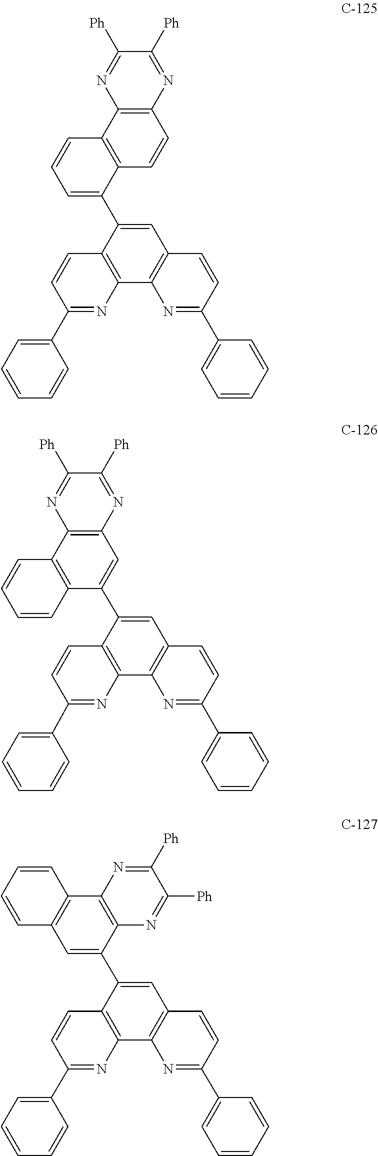 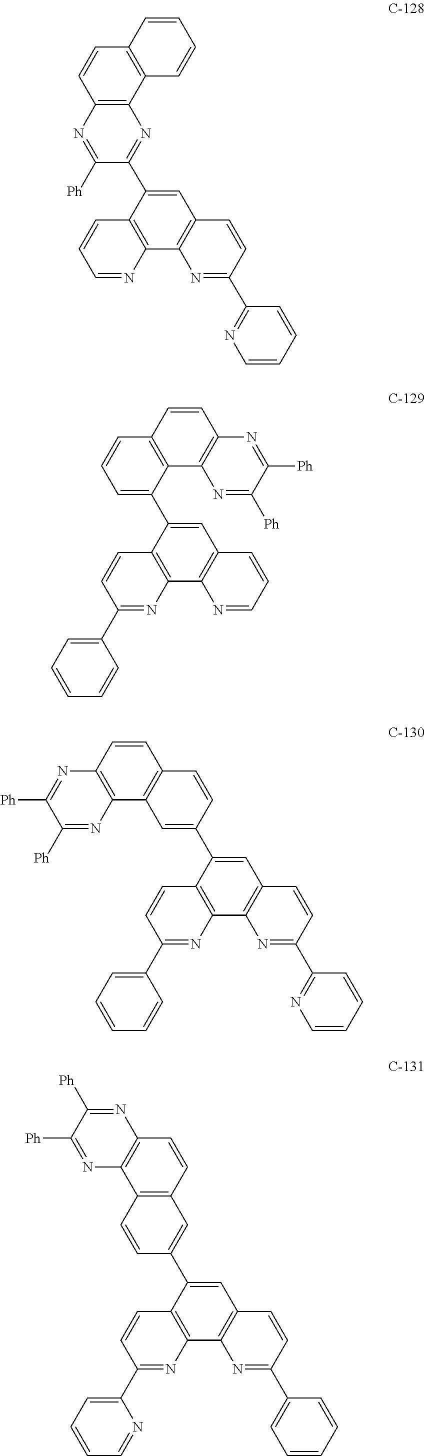 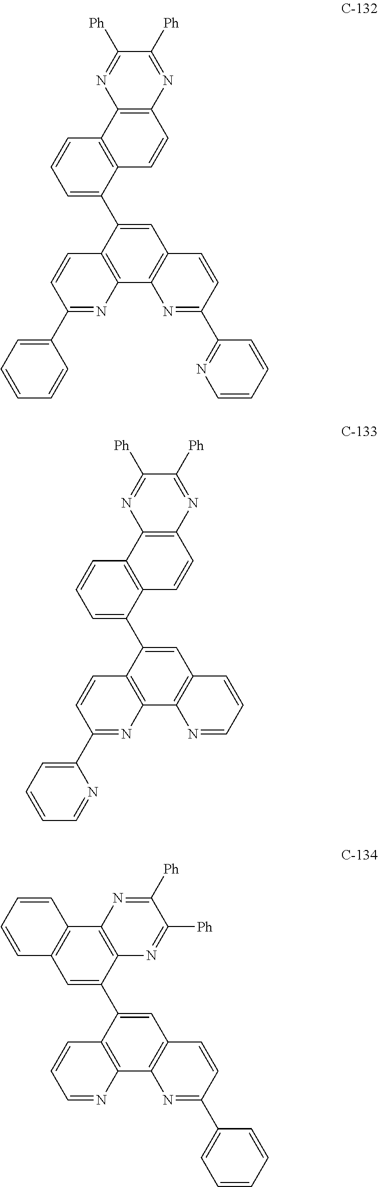 [SMILES: c1ccc(-c2ccc3c(-c4cccc5c4ccc4nc(-c6ccccc6)c(-c6ccccc6)nc45)cc4ccc(-c5ccccn5)nc4c3n2)cc1 ; c1ccc(-c2nc3ccc4c(-c5cc6cccnc6c6nc(-c7ccccn7)ccc56)cccc4c3nc2-c2ccccc2)cc1 ; c1ccc(-c2ccc3cc(-c4cc5ccccc5c5nc(-c6ccccc6)c(-c6ccccc6)nc45)c4cccnc4c3n2)cc1] 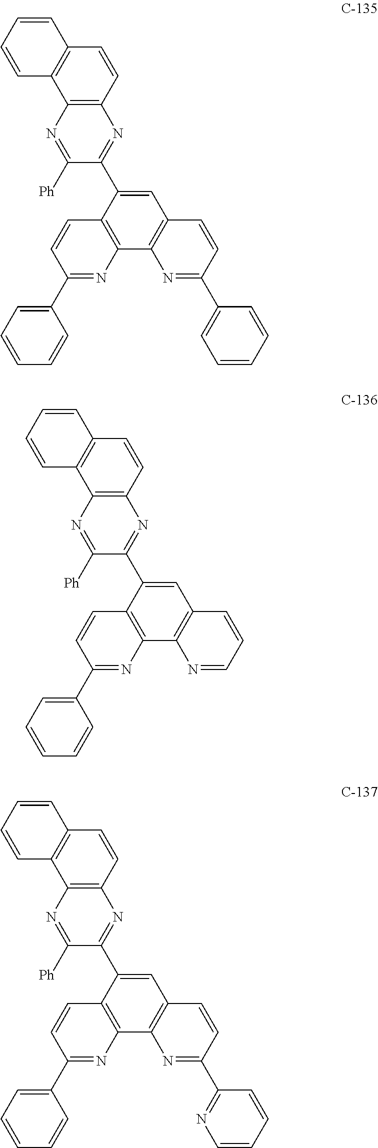 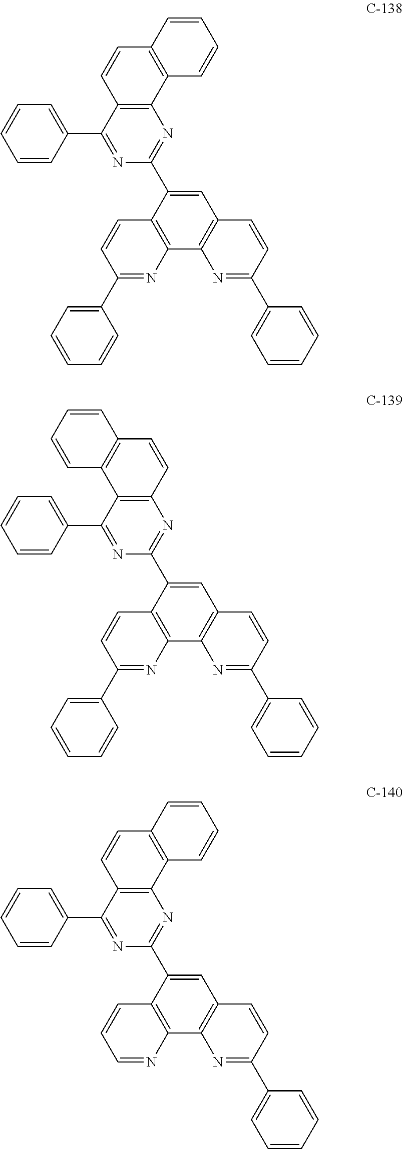 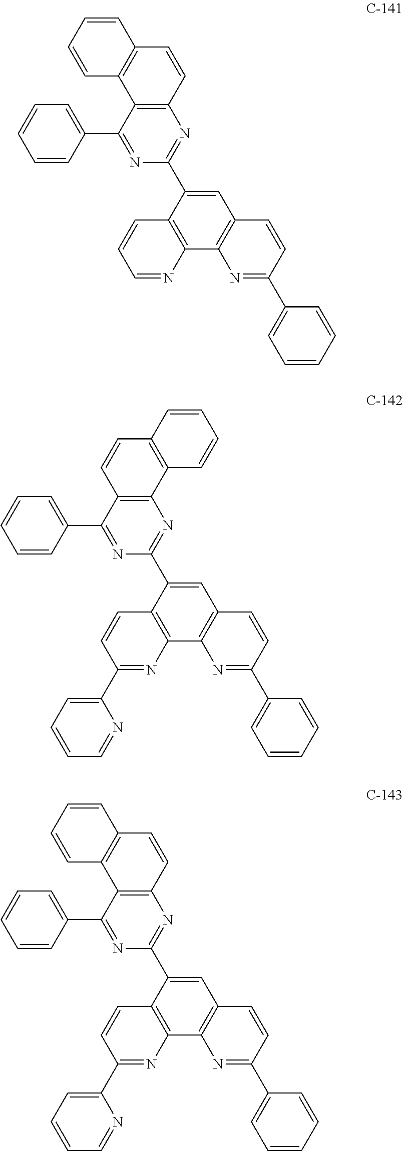 [SMILES: c1ccc(-c2ccc3cc(-c4nc(-c5ccccc5)c5c(ccc6ccccc65)n4)c4cccnc4c3n2)cc1 ; c1ccc(-c2ccc3cc(-c4nc(-c5ccccc5)c5ccc6ccccc6c5n4)c4ccc(-c5ccccn5)nc4c3n2)cc1 ; c1ccc(-c2ccc3cc(-c4nc(-c5ccccc5)c5c(ccc6ccccc65)n4)c4ccc(-c5ccccn5)nc4c3n2)cc1] 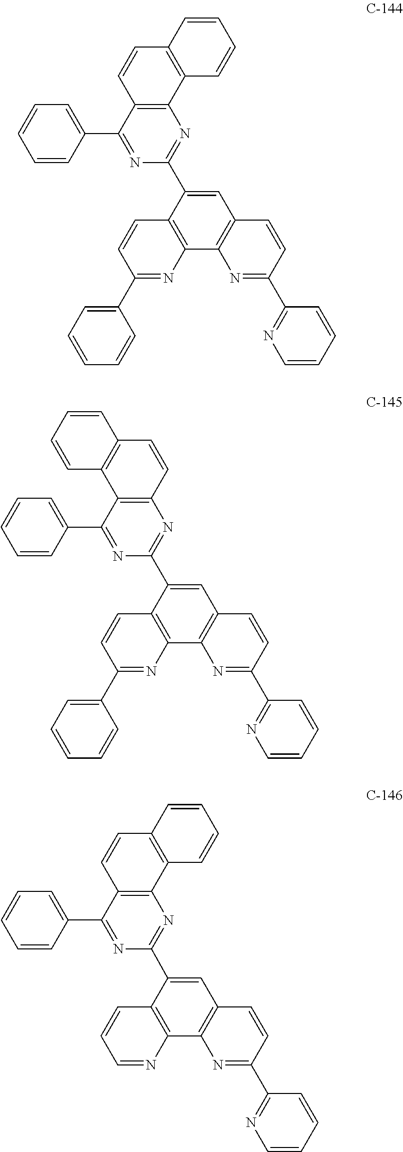 [SMILES: c1ccc(-c2ccc3c(-c4nc(-c5ccccc5)c5ccc6ccccc6c5n4)cc4ccc(-c5ccccn5)nc4c3n2)cc1 ; c1ccc(-c2ccc3c(-c4nc(-c5ccccc5)c5c(ccc6ccccc65)n4)cc4ccc(-c5ccccn5)nc4c3n2)cc1 ; c1ccc(-c2nc(-c3cc4ccc(-c5ccccn5)nc4c4ncccc34)nc3c2ccc2ccccc23)cc1] 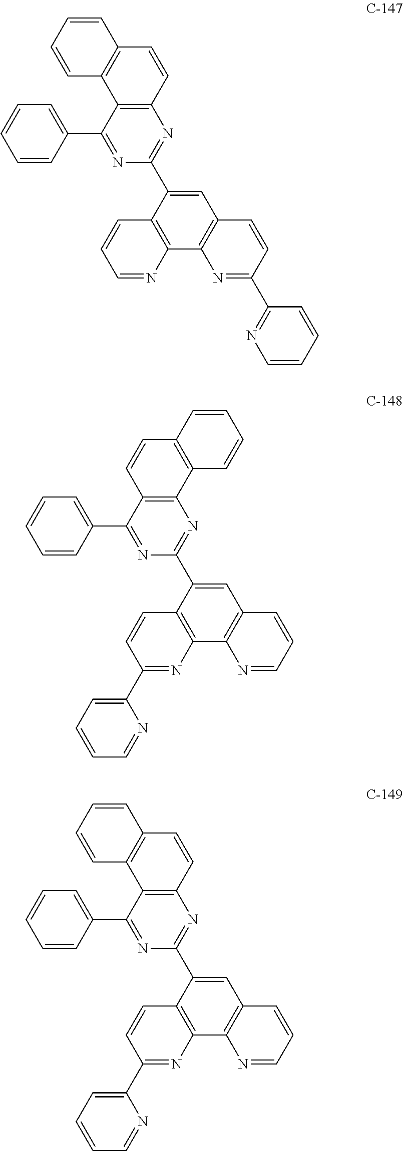 [SMILES: c1ccc(-c2nc(-c3cc4ccc(-c5ccccn5)nc4c4ncccc34)nc3ccc4ccccc4c23)cc1 ; c1ccc(-c2nc(-c3cc4cccnc4c4nc(-c5ccccn5)ccc34)nc3c2ccc2ccccc23)cc1 ; c1ccc(-c2nc(-c3cc4cccnc4c4nc(-c5ccccn5)ccc34)nc3ccc4ccccc4c23)cc1] 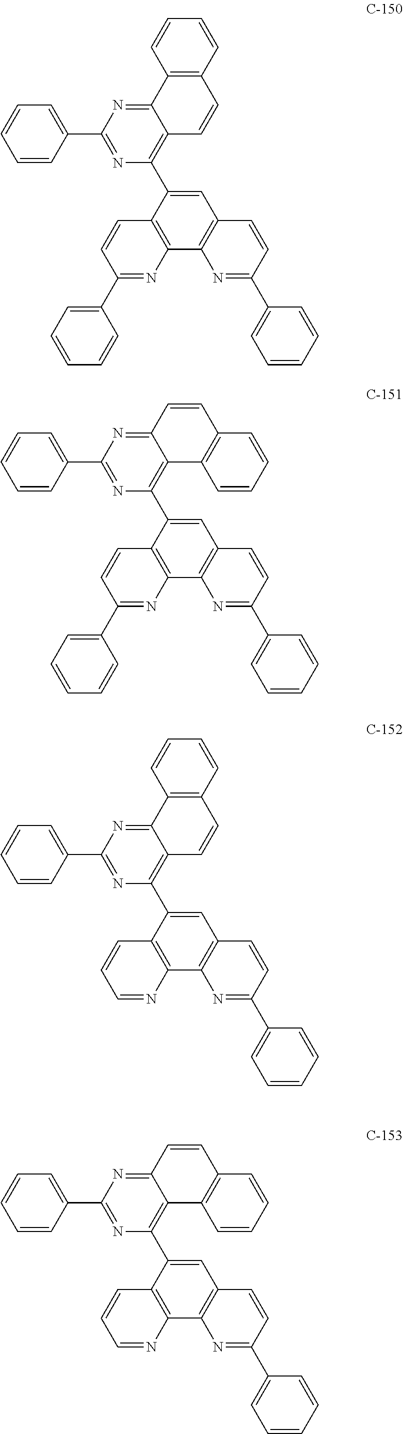 [SMILES: c1ccc(-c2ccc3cc(-c4nc(-c5ccccc5)nc5c4ccc4ccccc45)c4ccc(-c5ccccc5)nc4c3n2)cc1 ; c1ccc(-c2ccc3cc(-c4nc(-c5ccccc5)nc5ccc6ccccc6c45)c4ccc(-c5ccccc5)nc4c3n2)cc1 ; c1ccc(-c2ccc3cc(-c4nc(-c5ccccc5)nc5c4ccc4ccccc45)c4cccnc4c3n2)cc1 ; c1ccc(-c2ccc3cc(-c4nc(-c5ccccc5)nc5ccc6ccccc6c45)c4cccnc4c3n2)cc1] 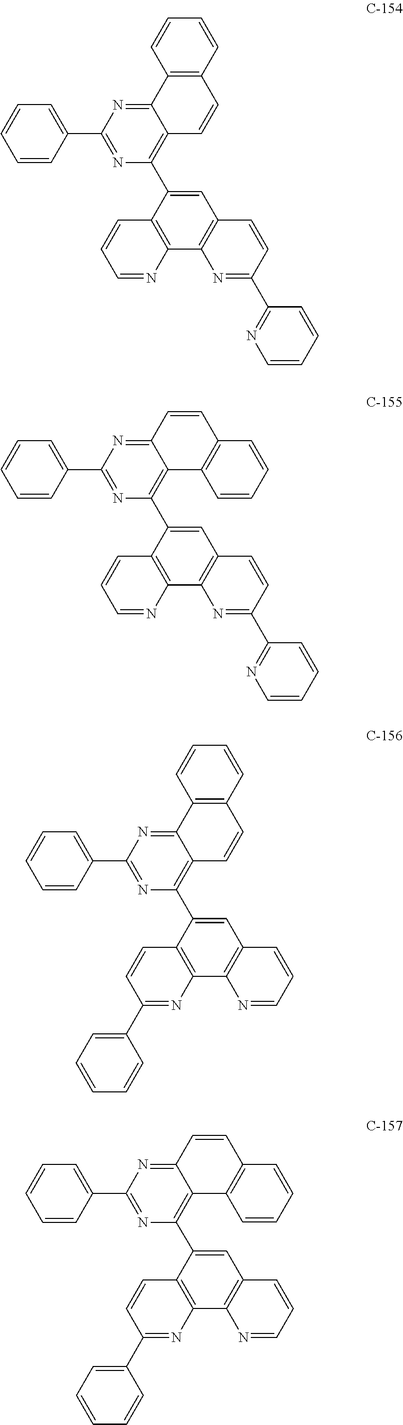 [SMILES: c1ccc(-c2nc(-c3cc4ccc(-c5ccccn5)nc4c4ncccc34)c3ccc4ccccc4c3n2)cc1 ; c1ccc(-c2nc(-c3cc4ccc(-c5ccccn5)nc4c4ncccc34)c3c(ccc4ccccc43)n2)cc1 ; c1ccc(-c2ccc3c(-c4nc(-c5ccccc5)nc5c4ccc4ccccc45)cc4cccnc4c3n2)cc1 ; c1ccc(-c2ccc3c(-c4nc(-c5ccccc5)nc5ccc6ccccc6c45)cc4cccnc4c3n2)cc1] 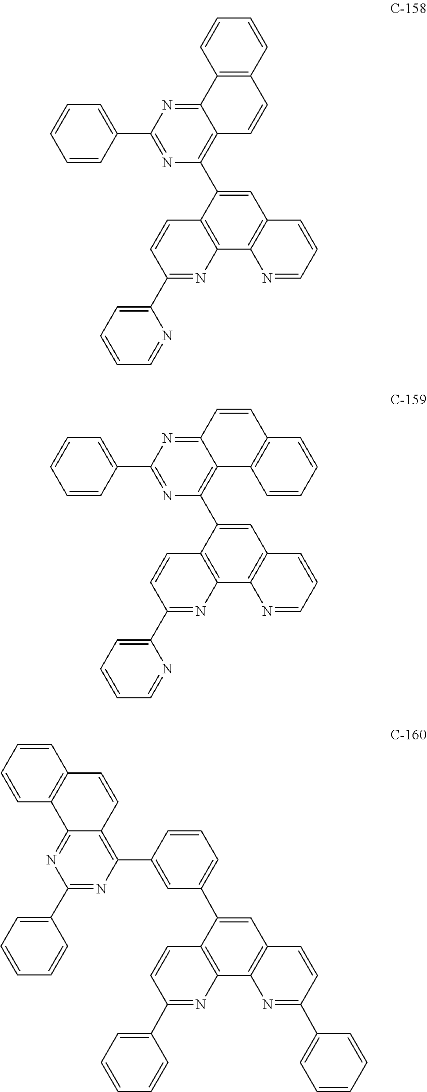 [SMILES: c1ccc(-c2nc(-c3cc4cccnc4c4nc(-c5ccccn5)ccc34)c3ccc4ccccc4c3n2)cc1 ; c1ccc(-c2nc(-c3cc4cccnc4c4nc(-c5ccccn5)ccc34)c3c(ccc4ccccc43)n2)cc1 ; c1ccc(-c2ccc3cc(-c4cccc(-c5nc(-c6ccccc6)nc6c5ccc5ccccc56)c4)c4ccc(-c5ccccc5)nc4c3n2)cc1] 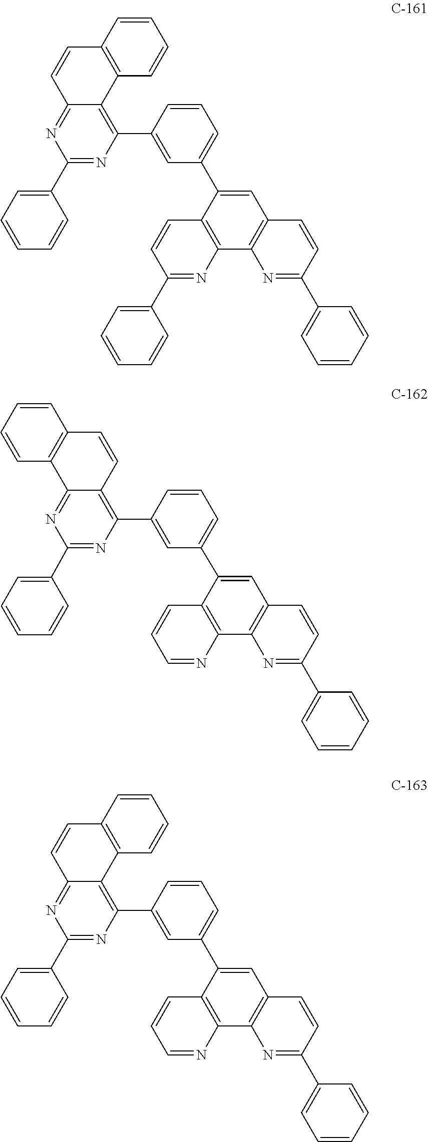 [SMILES: c1ccc(-c2ccc3cc(-c4cccc(-c5nc(-c6ccccc6)nc6ccc7ccccc7c56)c4)c4ccc(-c5ccccc5)nc4c3n2)cc1 ; c1ccc(-c2ccc3cc(-c4cccc(-c5nc(-c6ccccc6)nc6c5ccc5ccccc56)c4)c4cccnc4c3n2)cc1 ; c1ccc(-c2ccc3cc(-c4cccc(-c5nc(-c6ccccc6)nc6ccc7ccccc7c56)c4)c4cccnc4c3n2)cc1] 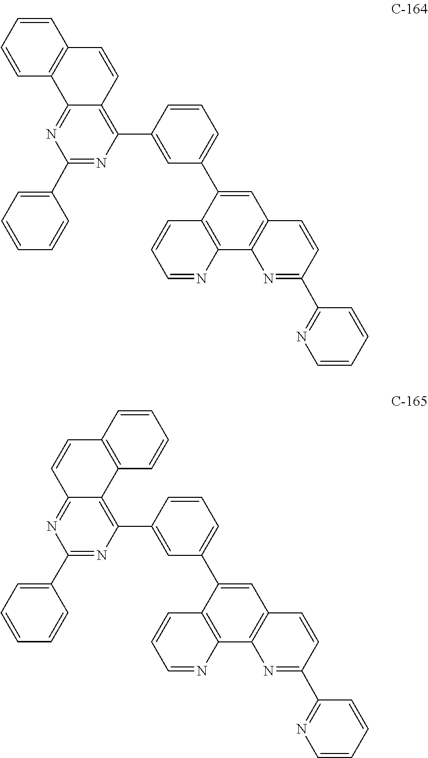 [SMILES: c1ccc(-c2nc(-c3cccc(-c4cc5ccc(-c6ccccn6)nc5c5ncccc45)c3)c3ccc4ccccc4c3n2)cc1 ; c1ccc(-c2nc(-c3cccc(-c4cc5ccc(-c6ccccn6)nc5c5ncccc45)c3)c3c(ccc4ccccc43)n2)cc1] 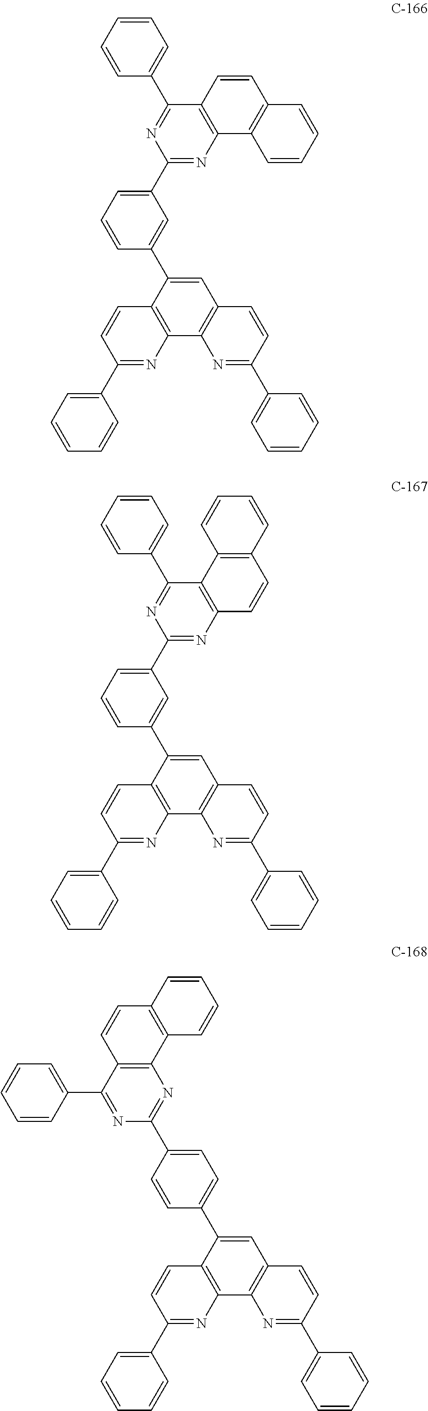 [SMILES: c1ccc(-c2ccc3cc(-c4cccc(-c5nc(-c6ccccc6)c6ccc7ccccc7c6n5)c4)c4ccc(-c5ccccc5)nc4c3n2)cc1 ; c1ccc(-c2ccc3cc(-c4cccc(-c5nc(-c6ccccc6)c6c(ccc7ccccc76)n5)c4)c4ccc(-c5ccccc5)nc4c3n2)cc1 ; c1ccc(-c2ccc3cc(-c4ccc(-c5nc(-c6ccccc6)c6ccc7ccccc7c6n5)cc4)c4ccc(-c5ccccc5)nc4c3n2)cc1] 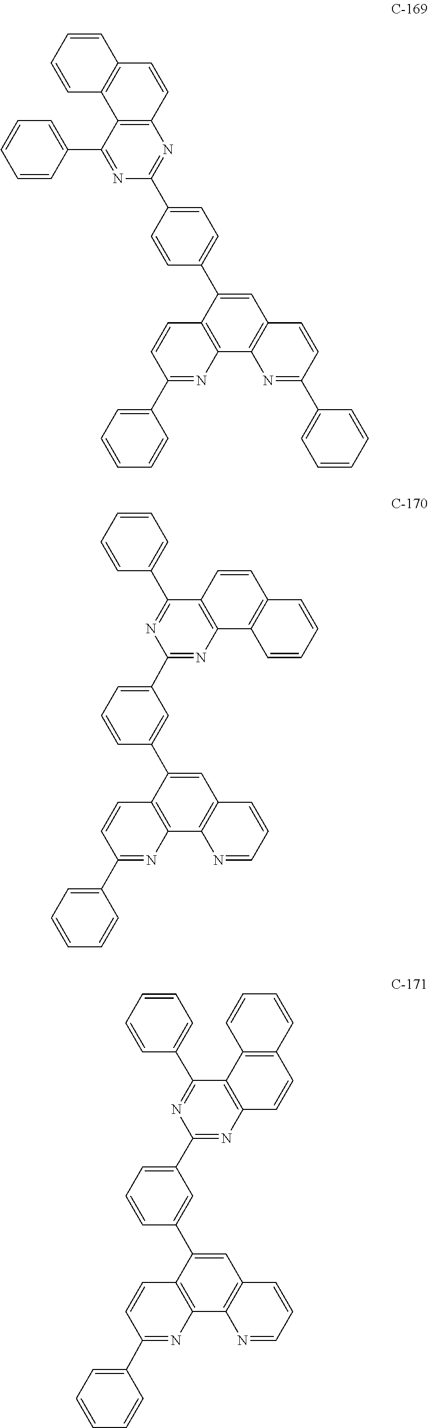 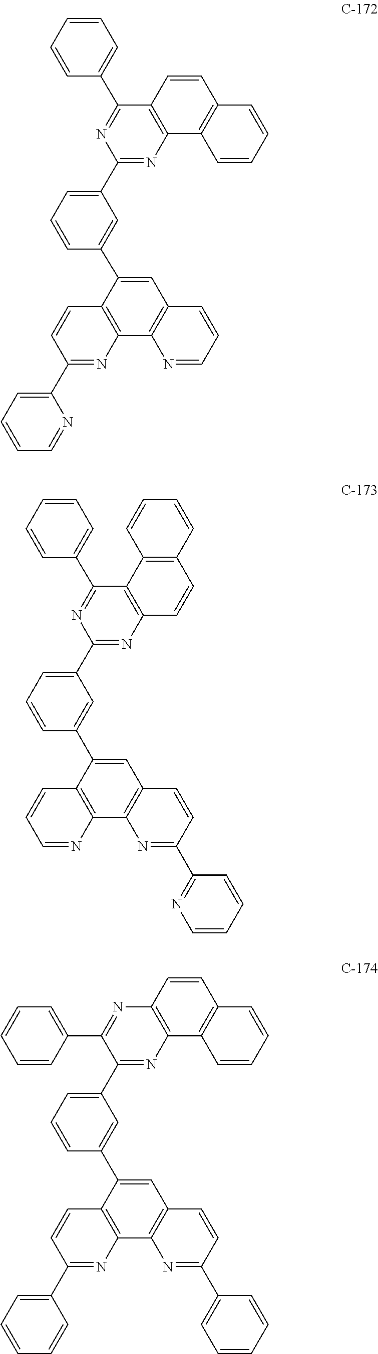 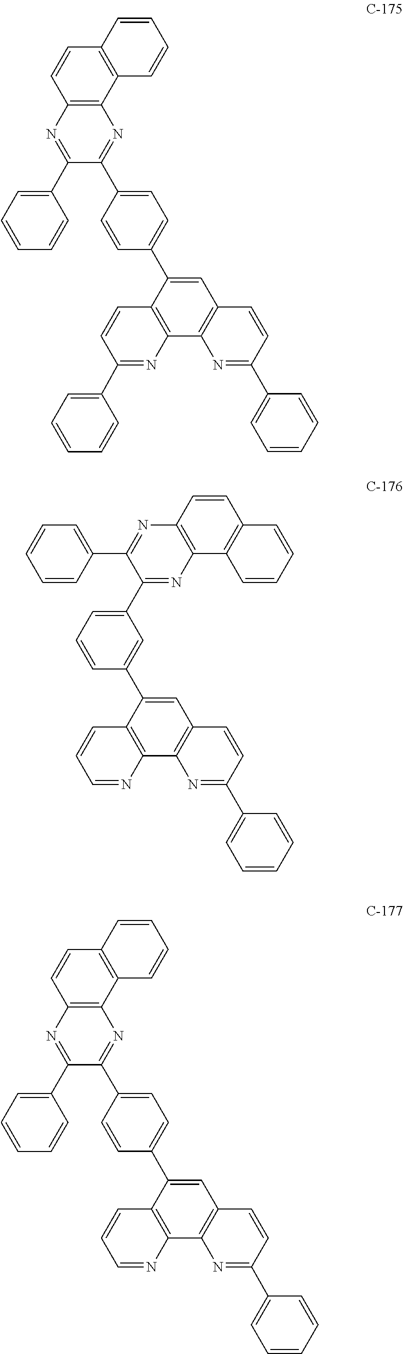 [SMILES: c1ccc(-c2ccc3cc(-c4ccc(-c5nc6c(ccc7ccccc76)nc5-c5ccccc5)cc4)c4ccc(-c5ccccc5)nc4c3n2)cc1 ; c1ccc(-c2ccc3cc(-c4cccc(-c5nc6c(ccc7ccccc76)nc5-c5ccccc5)c4)c4cccnc4c3n2)cc1 ; c1ccc(-c2ccc3cc(-c4ccc(-c5nc6c(ccc7ccccc76)nc5-c5ccccc5)cc4)c4cccnc4c3n2)cc1] 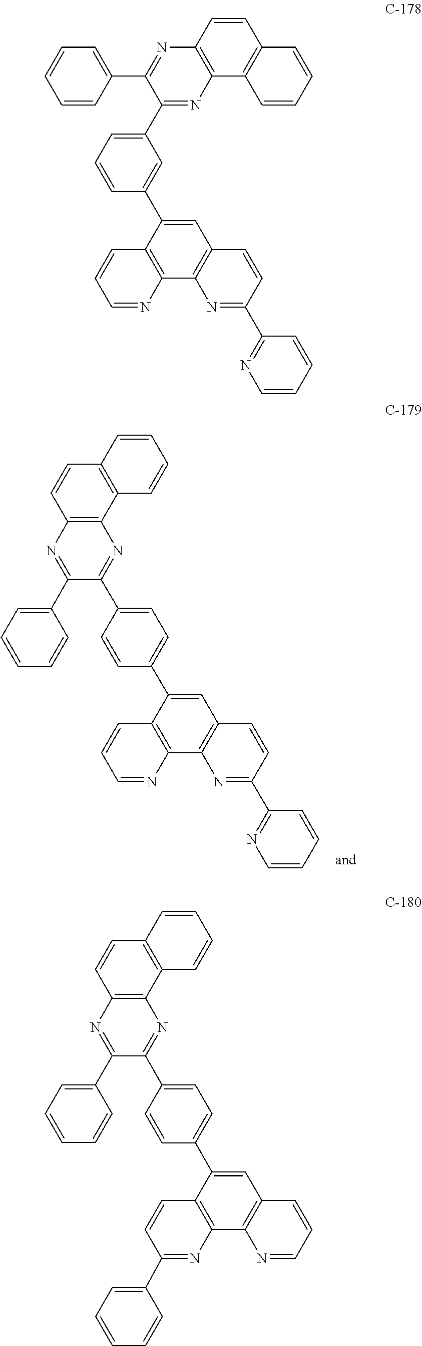 [SMILES: c1ccc(-c2nc3ccc4ccccc4c3nc2-c2cccc(-c3cc4ccc(-c5ccccn5)nc4c4ncccc34)c2)cc1 ; c1ccc(-c2nc3ccc4ccccc4c3nc2-c2ccc(-c3cc4ccc(-c5ccccn5)nc4c4ncccc34)cc2)cc1 ; c1ccc(-c2ccc3c(-c4ccc(-c5nc6c(ccc7ccccc76)nc5-c5ccccc5)cc4)cc4cccnc4c3n2)cc1]
In the compounds above, Dₙ means that n number of hydrogens are replaced with deuterium, and n represents an integer from 1 to the maximum number of hydrogens in the compound.

6. An organic electroluminescent material comprising the organic electroluminescent compound according to claim 1.

7. An organic electroluminescent device comprising the organic electroluminescent compound according to claim 1.

8. An N-type charge-generating material comprising the organic electroluminescent compound according to claim 1.

9. The organic electroluminescent device according to claim 7, wherein the organic electroluminescent device comprises a first electrode; a second electrode; a plurality of light-emitting units positioned between the first electrode and the second electrode; and at least one charge generation layer positioned between adjacent light-emitting units among the plurality of light-emitting units, wherein the charge generation layer comprises a compound in claim 1.
